(19) United States
(12) Patent Application Publication
(10) Pub. No.: US 2026/0071243 A1
(43) Pub. Date: Mar. 12, 2026

(54) BIOLOGICAL PRODUCTION OF TRYPTOPHAN-DERIVED PRODUCTS

(71) Applicant: BOARD OF REGENTS, THE UNIVERSITY OF TEXAS SYSTEM, Austin, TX (US)

(72) Inventors: Hal Alper, Austin, TX (US); Kevin Reed, Austin, TX (US); Sierra Brooks, San Francisco, CA (US)

(21) Appl. No.: 19/327,017

(22) Filed: Sep. 12, 2025

Related U.S. Application Data
(60) Provisional application No. 63/693,885, filed on Sep. 12, 2024.

(51) Int. Cl. C12P 13/22; C12N 9/02; C12N 9/06

(52) U.S. Cl. CPC C12P 13/22; C12N 9/0028; C12N 9/0071; C12Y 105/0103

(57) ABSTRACT

A consortium of engineered microorganisms for producing tryptophan-derived products and methods of using the same.

### CROSS-REFERENCE TO RELATED APPLICATIONS

This application claims the benefit of priority to U.S. Provisional Application No. 63/693,885, filed Sep. 12, 2024, which is incorporated by reference herein in its entirety.

### GOVERNMENT SUPPORT CLAUSE

This invention was made with government support under Grant No. HR0011-15-9-0014 awarded by the Defense Advanced Research Projects Agency (DARPA). The Government has certain rights in the invention.

### REFERENCE TO SEQUENCE LISTING

The sequence listing submitted on Sep. 12, 2025, as an .XML file entitled “10046-622US1_ST26.xml” created on Sep. 10, 2025, and having a file size of 97,081 bytes is hereby incorporated by reference pursuant to 37 C.F.R. § 1.52(e)(5).

### BACKGROUND

The vast majority of biochemistry revolves around a finite number of elements on the periodic table. However, by coupling synthetic tools with the exploration of diverse microbial biochemical space, new pathways for the assimilation of more uncommon elements are emerging [1][2][3]. This expanded scope is moving metabolic engineering away from simply C—C, C—H, C—O, C—N, and C—S bonds and toward alternative chemical diversity, thus expanding the cellular chemical palette [1][4][5]. Among these alternative pathways of interest is the incorporation of halogen elements into microbial metabolism [6][7][8][9][10]. Halogenation chemistry has become rather ubiquitous in modern society with broad applications spanning pharmaceuticals [11][12][13][14], agrochemicals [15][16][17], and other materials [18][19][20][21]. The critical incorporation of a halogen atom into compounds directly confers function as seen in examples such as the widely used polymer polyvinylchloride (PVC), the potent antibiotic chloramphenicol, and the flame retardant brominated polystyrene (BPS) [22]. Organic synthesis of halogenated compounds, while well-established and understood, is marred by highly toxic chemicals [15], poor atom economy [23], and limited stereo-/regio-selectivity [24] that hinders targeted halogenation in complex molecules [25]. Furthermore, the requirement for enantiomerically pure end products often complicates separation and purification chemical synthesis of these compounds [26]. In contrast, biological synthesis routes can bypass many of these limitations and theoretically offer an effective and more environmentally friendly alternative for producing halogenated molecules at near ambient conditions. Thus, there is a need in the art for biological production of halogenated molecules.

### SUMMARY

Disclosed herein is a platform strain for the incorporation of halogenated (i.e., chlorine (Cl) and bromine (Br)) incorporated biomolecules that stem from tryptophan. A major approach here is a modular co-culture fermentation which uses a plug-and-play bioproduction platform using a promiscuous downstream enzyme. This can culminate in the generation of at least 26 distinct halogenated molecules, including precursors to prodrugs 4-chloro- and 4-bromo-kynurenine and halogenated beta carbolines. Precursor strains containing chloro- and bromo-modified tryptophan (in multiple positions) are demonstrated through strain engineering which are capable of producing between 300 mg/L and 700 mg/L. The disclosed strains create a platform for bioproduction of these halogenated compounds. Biological production of halogenated compounds can be conducted in far more benign conditions than chemical routes, and can provide greater product specificity. Biological production provides a sustainable, green, and safe manufacturing of these molecules. Strain engineering can also further improve production.

In one aspect, provided is a consortium of engineered microorganisms, including: at least one upstream engineered microorganism for producing halogenated tryptophan; and at least one downstream engineered microorganism for converting said halogenated tryptophan into a tryptophan-derived product.

In another aspect, provided is a method of making a tryptophan-derived product, the method including: a) providing any of the disclosed consortia; b) exposing the at least one upstream engineered microorganism to a feedstock, thereby producing a halogenated tryptophan; and c) exposing the at least one downstream engineered microorganism to the halogenated tryptophan, thereby converting the halogenated tryptophan to a tryptophan-derived product.

In yet another aspect, provided is a tryptophan-derived product generated by any of the disclosed methods.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRAWINGS

FIGS. 1A-1C depict the selection of halogenases to acquire a panel of modified precursors. FIG. 1A shows a reaction overview for tryptophan halogenases. 5-tryptophan-halogenases: PyrH, XsHal; 6-tryptophan-halogenases: Th-Hal, SttH, Thal; 7-tryptophan-halogenases: RebH, PrnA. FIG. 1B shows the experimental design for the halogenase panel. Strain sKR-160 containing respective halogenases and flavin reductase Th-Fre were supplied with 1 mM of tryptophan and production formation of respective halogenated tryptophan products (5-, 6-, 7-chloro-/bromo-tryptophan) was observed. FIG. 1C shows a panel of halogenases with calculated L-tryptophan conversion at varying temperatures: 25° C., 30° C., and 37° C. Data are mean±S.D.; n=3 biological replicates.

FIGS. 2A-2B depict production of 6-chloro-tryptophan (6-Cl-Trp) via varied expression regimes. Strain sKR-TrpO harboring plasmids with varying expression regimes for halogenase Th-Hal and flavin reductases Fre and Th-Fre were grown for 24-hour incubation with 1 mM of L-tryptophan fed in biological triplicate. Error bars represent S.E. of n=3 biological triplicates.

FIG. 3 depicts residual chloro-tryptophan from the halogenase panel. Percentage of chloro-tryptophan in final production for halogenase panel with media containing primarily NaBr salt. Error bars represent S.E. of n=3 biological replicates.

FIGS. 4A-4B depict metabolic engineering in E. coli for L-tryptophan production. FIG. 4A shows genetic changes leading to high tryptophan production in E. coli BW25113 background and schematic of the tryptophan overproduction strategy for strain sKR-Trp4, the highest producer of tryptophan from glucose generated in this study. The outer loop within the bottom schematic of FIG. 4A represents the genome and relevant genomic modifications, including deletions of TrpR and TnaA and the integration of a precursor module containing SerA(fbr) and AroG(fbr) within the rbsAR locus. Glucose 6-P: glucose-6-phosphate; PP Pathway: pentose phosphate pathway; PEP: phosphoenolpyruvate; E4P: erythrose-4-phosphate; DAHP: 3-deoxy-D-arabino-heptulosonic acid 7-phosphate; DHQ: 3-dehydroquinate; DHS: 3-dehydroshikimate; S3P: shikimate-3-phosphate; EPSP: 5-enolpyruvylshikimate 3-phosphate; Ant: anthranilate; L-Trp: L-tryptophan; 3-PDG: 3-phospho-D-glycerate. FIG. 4B shows tryptophan overproduction based on genetic modifications outlined in TABLE 3 and TABLE 5. Each bar represents a biological triplicate for fermentations of 5 g/L glucose in minimal media after 24 h. Data are mean±S.D.; n=3 biological replicates.

FIGS. 5A-5B depict de novo production of halogenated tryptophan in E. coli. FIG. 5A shows a schematic of final halogenated tryptophan overproduction strain, showing modular engineering. Hal: halogenase (XsHal, Thal, or RebH depending on the position of interest); Fl-Red: flavin reductase. FIG. 5B shows reaction overviews of each introduced halogenase and corresponding production curves when fed with 40 g/L glucose for 72 h. Data are mean±S.D.; n=3 biological replicates.

FIGS. 6A-6C depict a comparison of media formulations for production of bromotryptophan. M9G is conventional M9 media with 0.4% glucose. Excess NaBr (250 mM) was added to this media. In comparison, media denoted M9G-Br has the typical formulation of M9 however all the chloride salts replaced with bromide salts (ammonium bromide, sodium bromide, etc.). An additional 100 mM NaBr was added to this media. Error bars represent S.E. of n=3 biological replicates.

FIGS. 7A-7D depict establishing downstream pathways to access molecules from tryptophan. FIG. 7A shows an overview of example molecules derived from tryptophan and the applications enabled by them. Molecules in the figure (in general order left to right) include Indole, Kynurenine, Kynurenic acid, Skatole, 3-methyl-2-indolic acid (MIA), Serotonin, Indole-3-acetic acid (auxin), Tryptamine, indole pyruvic acid, 1-acetyl-3-carboxy-beta-carboline, strictosidine, indigo, violacein, rebeccamycin, thaxtomin, vinblastine, cyclomarin A, ergoline, ergotamine, N,N-Dimethyltryptamine, Hapalindole A, melatonin, psilocybine, lysergic acid, physostigmine, pyrrolnitrin, and quinmerac. FIG. 7B shows biologically available reaction centers of tryptophan investigated within this study accessible through a single enzymatic step. Note: This list is exemplary, not exhaustive. The modifications to the tryptophan scaffold are represented by colored reaction center dots, with colors corresponding to each reaction center displayed in FIG. 7C. The green boxes in the product observed column represent the enzymes that converted tryptophan fed to the media into the expected product, as confirmed by LC-MS. FIG. 7C shows an overview of the halogen-product diversification strategy, utilizing promiscuity downstream enzymes to generate a wide range of halogenated tryptophan-derived products. FIG. 7D shows the chemical structure of the expected products, with indications of observed and unobserved when 500 M L-tryptophan was fed to the media.

FIGS. 8A-8B depict the evaluation of downstream promiscuity through feeding assays. FIG. 8A shows the reaction overview for each downstream enzyme evaluated. FIG. 8B shows the results of each strain containing the corresponding downstream enzyme fed 500 M of each halogenated tryptophan analog. Each heat map square represents the formation (green) or lack of formation (white) of corresponding halogenated products from biological triplicates picked from individual colonies.

FIG. 9 depicts an overview of products investigated in this study. Molecule map containing all the theoretical molecules to be fed or produced from the six functioning downstream enzymes evaluated in this study, organized by functional group substitution (halogen) and base molecule. Molecule names are as follows, emphasizing the halogen first: tryptophan (1a), 5-chloro-tryptophan (1b), 5-bromo-tryptophan (1c), 6-chloro-tryptophan (1d), 6-bromo-tryptophan (1e), 7-chloro-tryptophan (1f), 7-bromo-tryptophan (1g), tryptamine (2a), 5-chloro-tryptamine (2b), 5-bromo-tryptamine (2c), 6-chloro-tryptamine (2d), 6-bromo-tryptamine (2e), 7-chloro-tryptamine (2f), 7-bromo-tryptamine (2g), indole-3-acetamide (3a), 5-chloroindole-3-acetamide (3b), 5-bromo-indole-3-acetamide (3c), 6-chloro-indole-3-acetamide (3d), 6-bromo-indole-3-acetamide (3e), 7-chloro-indole-3-acetamide (3f), 7-bromo-indole-3-acetamide (3g), indole (4a), 5-chloro-indole (4b), 5-bromo-indole (4c), 6-chloro-indole (4d), 6-bromoindole (4e), 7-chloro-indole (4f), 7-bromo-indole (4g), N-formyl-L-kynurenine (5a), 5-chloro-N-formyl-L-kynurenine (5b), 5-bromo-N-formyl-L-kynurenine (5c), 6-chloro-N-formyl-L-kynurenine (5d), 6-bromo-N-formyl-L-kynurenine (5e), 7-chloro-N-formyl-L-kynurenine (5f), 7-bromo-N-formyl-L-kynurenine (5g), 1-acetyl-3-carboxy-β-carboline (6a), 5-chloro-1-acetyl-3-carboxy-β-carboline (6b), 5-bromo-1-acetyl-3-carboxy-β-carboline (6c), 6-chloro-1-acetyl-3-carboxy-β-carboline (6d), 6-bromo-1-acetyl-3-carboxy-β-carboline (6e), 7-chloro-1-acetyl-3-carboxy-β-carboline (6f), 7-bromo-1-acetyl-3-carboxy-β-carboline (6g), 2-methyl-L-tryptophan (7a), 5-chloro-2-methyl-L-tryptophan (7b), 5-bromo-2-methyl-L-tryptophan (7c), 6-chloro-2-methyl-L-tryptophan (7d), 6-bromo-2-methyl-L-tryptophan (7e), 7-chloro-2-methyl-L-tryptophan (7f), 7-bromo-2-methyl-L-tryptophan (7g).

FIGS. 10A-10B depict confirmation of pharmaceutically relevant molecules through microbial fermentation. FIG. 10A shows confirmation of prodrug precursors 6-chloro-N-formyl-L-kynurenine (blue) and 6-bromo-N-formyl-L-kynurenine (orange). FIG. 10B shows confirmation of new-to-nature molecules 7-chloro-1-acetyl-3-carboxy-β-carboline (blue) and 7-bromo-1-acetyl-3-carboxy-β-carboline (orange).

FIG. 11 depicts modular one-pot de novo co-culture reactions enable halogenated product diversity. Shown is a schematic of the modular one-pot de novo co-culture reactions to enable halogenated product diversity. One strain converts glucose into 5-, 6-, or 7-halogenated tryptophan. The second strain converts the halogenated tryptophan into a halogenated downstream product which is secreted into the media. Boxes with both Br and Cl icons refer to downstream products for which both brominated and chlorinated versions have been detected using each respective downstream enzyme. Boxes without Br and Cl refer to downstream products which were not detected when attempting the specified co-culture.

FIG. 12 depicts tryptamine quantification in halo-tryptamine co-culture. Production of tryptamine using A) only the downstream module cell, B) upstream plus downstream co-culture in chloride-focused media, and C) upstream plus downstream co-culture in bromide-focused media. Error bars represent S.E. of 3 biological triplicates.

FIG. 13 depicts halo-tryptamine quantification in halo-tryptamine co-culture. Production of 5-Cl-tryptamine and 5-Br-tryptamine using A) only the downstream module cell, B) upstream plus downstream co-culture in chloride-focused media, and C) upstream plus downstream co-culture in bromide-focused media. Error bars represent S.E. of 3 biological triplicates.

FIGS. 14A-14C depict de novo production of a wide range of tryptophan-derived halogenated products through modular one-pot co-cultures. FIG. 14A depicts production of chloro-specific products, where each heat map square represents the formation (blue) or lack of formation (white) of corresponding halogenated products from biological triplicates picked from individual colonies. New-to-nature molecules are designated with a black star and products produced via de novo microbial synthesis for the first time are designated with a black circle. FIG. 14B depicts production of bromo-specific products, where each heat map square represents the formation (orange) or lack of formation (white) of corresponding halogenated products from biological triplicates picked from individual colonies. New-to-nature molecules are designated with a black star and products produced by microbial synthesis are designated with a black circle.

FIG. 14C depicts confirmation of de novo production of 6-chloro-N-formyl-L-kynurenine (5d), 6-bromo-N-formyl-L-kynurenine (5e), 7-chloro-1-acetyl-3-carboxy-β-carboline (6f), 7-bromo-1-acetyl-3-carboxy-β-carboline (6g), respectively, via LC-MS.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

In this specification and in the claims that follow, reference will be made to a number of terms, which shall be defined to have the following meanings:

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a strain”, includes, but is not limited to, two or more such compounds, compositions, or strains, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

The term “engineered” and “modified” are used interchangeably to refer to microorganisms which comprise at least one element which differs from the naturally occurring microorganism. The microorganism can be genetically engineered to express, or over-express, a moiety not normally present, or expressed in different amounts, in a naturally occurring microorganism, for example, which has not been modified. Alternatively, or also, the engineered microorganism can be genetically engineered to not express, or have reduced expression, of a moiety which is normally present, or present in different amounts, in a naturally occurring microorganism. It is noted that the engineered microorganism need not be genetically modified to be considered “engineered” or “modified.” For example, the microorganism can comprise a moiety, such as a synthetic payload, which has been introduced to the microorganism by means other than genetically. For example, the microorganism can be induced to take up a synthetic payload.

The term “consortia” or “consortium” refers to a subset of a microbial community of individual microbial species, or strains of a species, which can be described as participating in, or leading to, or correlating with, a recognizable parameter, such as a phenotypic trait of interest or common function.

As used herein, a microorganism can be said to “convert” a first moiety to a second moiety if, after a given duration, the first moiety is transformed into the second moiety. It is understood that the microorganism need not transform 100% of a given amount of the first moiety into the second moiety to be considered capable of “converting” the first moiety to the second moiety. For example, the microorganism can be said to “convert” the first moiety to the second moiety if, after a given duration, the microorganism can transform at least about 5% (e.g., at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, about 100%) of a given amount the first moiety into the second moiety. It is also understood that the ability of a microorganism to convert the first moiety into the second moiety may vary with time or environmental factors (e.g., temperature, concentration of the first moiety, concentration of the second moiety, presence or absence of additional moieties, etc.).

### Consortia

In an aspect, provided is a consortium of engineered microorganisms, including: at least one upstream engineered microorganism for producing halogenated tryptophan; and at least one downstream engineered microorganism for converting said halogenated tryptophan into a tryptophan-derived product. An example of this consortium can be seen in FIG. 11. It is understood that terms “upstream” and “downstream” refer to the relative order of operations in a synthesis or signaling pathway. An “upstream” step or component will occur before a “downstream” step or component, either immediately before, or with one or more intermediate steps or components between the upstream and downstream steps or components. Generally, an output of an “upstream” step or component will influence the input of a “downstream” step or component. As a specific example, in the consortium disclosed herein, the “upstream” microorganism outputs halogenated tryptophan, and then, subsequently, the “downstream” microorganism converts this halogenated tryptophan into a tryptophan-derived product.

In some aspects, the at least one upstream engineered microorganism can convert a carbon source to tryptophan and subsequently convert said tryptophan to halogenated tryptophan. In other aspects, a first upstream engineered microorganism can convert a carbon source to tryptophan; and a second upstream engineered microorganism can subsequently convert said tryptophan to halogenated tryptophan.

As used herein, the term “carbon source” refers to a substrate or compound suitable to be used as a source of carbon to generate tryptophan. Carbon sources can be in various forms, including, but not limited to polymers, carbohydrates, acids, alcohols, aldehydes, ketones, amino acids, peptides, and gases (e.g., CO and CO₂). Exemplary carbon sources include, but are not limited to, monosaccharides, such as glucose, fructose, mannose, galactose, xylose, and arabinose; oligosaccharides, such as fructo-oligosaccharide and galacto-oligosaccharide; polysaccharides such as starch, cellulose, pectin, and xylan; disaccharides, such as sucrose, maltose, cellobiose, and turanose; cellulosic material and variants such as hemicelluloses, methyl cellulose and sodium carboxymethyl cellulose; saturated or unsaturated fatty acids, succinate, lactate, and acetate; alcohols, such as ethanol, methanol, and glycerol, or mixtures thereof. The carbon source can also be a product of photosynthesis, such as glucose. In some specific aspects, the carbon source can be biomass. In other specific aspects, the carbon source can be glucose. In yet other specific aspects, the carbon source can be sucrose.

As used herein, the term “biomass” refers to any biological material from which a carbon source is derived. In some aspects, a biomass can be processed into a carbon source, which is suitable for bioconversion. In other aspects, the biomass may not require further processing into a carbon source. An exemplary source of biomass is plant matter or vegetation, such as corn, sugar cane, or switchgrass. Another example source of biomass is metabolic waste products, such as animal matter (e.g., cow manure). Further example sources of biomass include algae and other marine plants. Biomass also includes waste products from industry, agriculture, forestry, and households, including, but not limited to, fermentation waste, ensilage, straw, lumber, sewage, garbage, cellulosic urban waste, and food leftovers. The term “biomass” also refers to sources of carbon, such as carbohydrates (e.g., monosaccharides, disaccharides, or polysaccharides).

In some aspects, the at least one upstream engineered microorganism can express at least one halogenase. In some aspects, the at least one halogenase can be a tryptophan 5-halogenase, a tryptophan 6-halogenase, a tryptophan 7-halogenase, a tryptophan 5,7-halogenase, or any combination thereof. For example, in some aspects, the at least one halogenase can be tryptophan 5-halogenase from Streptomyces rugosporus (PyrH), tryptophan 5-halogenase from Xenorhabdus szentirmaii (XsHal), tryptophan 6-halogenase from Streptomyces albogriseolus (Thal), tryptophan 6-halogenase from Streptomyces violaceusniger (Th-Hal), tryptophan 6-halogenase from Streptomyces toxytricini (SttH), tryptophan 7-halogenase from Lechevalieria aerocolonigenes (RebH), tryptophan 7-halogenase from Pseudomonas fluorescens (PrnA), tryptophan 5,7-halogenase (AetF), or any combination thereof.

In some aspects, the at least one upstream engineered microorganism can further express an enzyme for generating a cofactor for the at least one halogenase. For example, in some aspects, the cofactor can be flavin adenine dinucleotide (FADH₂), and the enzyme for generating the cofactor can be a flavin reductase. In some such aspects, the flavin reductase can be sourced from any suitable organism, for example, an E. coli flavin reductase or a flavin reductase from any other suitable organism. In some aspects, the flavin reductase can be thermostable.

In some aspects, the halogenated tryptophan can be chlorinated or brominated. For example, the halogenated tryptophan can include 5-chloro-tryptophan, 6-chloro-tuyptophan, 7-chloro-tryptophan, 5,7-dichloro-tryptophan, 5-bromo-tryptophan, 6-bromo-tryptophan, 7-bromo-tryptophan, 5,7-dibromo-tryptophan, or any combination thereof.

In some aspects, the at least one downstream engineered microorganism can express at least one downstream enzyme. In some such aspects, the at least one downstream enzyme can be promiscuous. A “promiscuous” enzyme is understood to be capable of acting on multiple different substrates, whether native or non-native.

In some aspects, a first downstream enzyme can convert the halogenated tryptophan into a halogenated intermediate; and a second downstream enzyme can convert the halogenated intermediate into the tryptophan-derived product. In some such aspects, the first downstream enzyme can be RgnT, RgnTD, or any combination thereof; and the second downstream enzyme can be RgnDC, RgnC, or any combination thereof.

In other aspects, the at least one downstream enzyme can directly convert the halogenated tryptophan to the tryptophan-derived product. In some such aspects, the at least one downstream enzyme can be iaaM, TnaA, KynA, McbB, or any combination thereof.

In some aspects, the tryptophan-derived product can be halogenated. For example, in some aspects, the tryptophan-derived product can include halo-tryptamine, halo-indole-3-acetamide, halo-indole, halo-N-formyl-L-kynurenine, halo-1-acetyl-3-carboxy-β-carboline, halo-2-methyl-L-tryptophan, or any combination thereof.

In other aspects, the tryptophan-derived produced may not halogenated. For example, in some aspects, the tryptophan-derived product can include ryptamine, indole-3-acetamide, indole, N-formyl-L-kynurenine, 1-acetyl-3-carboxy-β-carboline, 2-methyl-L-tryptophan, N-dimethylallyl-L-tryptophan, 4-dimethylallyl-L-tryptophan, 7-dimethylallyl-L-tryptophan, indole-3-acetaldoxime, kynurenine, kynurenic acid, skatole, 3-methyl-2-indolic acid (MIA), serotonin, indole-3-acetic acid (auxin), indole pyruvic acid, strictosidine, indigo, violacein, rebeccamycin, thaxtomin, vinblastine, cyclomarin A, ergoline, ergotamine, N,N-dimethyltryptamine, hapalindole A, melatonin, psilocybine, lysergic acid, physostigmine, pyrrolnitrin, qluinnerac, or any combination thereof.

In yet other aspects, the tryptophan derived-product can include a combination of halogenated and non-halogenated molecules, for example, any of the halogenated and non-halogenated molecules described above.

In some aspects, each of the at least one upstream engineered microorganism and the at least one downstream engineered microorganism can be independently selected from a yeast or a bacterium. In some aspects, the yeast can be Saccharomyces. In some aspects, the bacterium can be E. coli or C. glutamicum.

In some aspects, the at least one upstream engineered microorganism and the at least one downstream engineered microorganism can be co-cultured. As used herein, the term “co-culture” refers to culturing two or more microorganisms such that each microorganism can receive signals and/or inputs from the other (e.g., in the same culture dish or in separate culture dishes which are at least partially fluidically connected) In other aspects, the at least one upstream engineered microorganism and the at least one downstream engineered microorganism can be separately cultured. It is considered that, in some specific aspects, the separation of the at least one upstream engineered microorganism from the at least one downstream engineered microorganism can allow a promiscuous downstream enzyme to produce a broader array of tryptophan-derived products and also allow for a more facile mixing-and-matching of the upstream and downstream components to generate a wider variety of tryptophan-derived products.

### Methods and Products

In an aspect, provided is a method of making a tryptophan-derived product, the method including: a) providing any of the disclosed consortia; b) exposing the at least one upstream engineered microorganism to a feedstock, thereby producing a halogenated tryptophan; and c) exposing the at least one downstream engineered microorganism to the halogenated tryptophan, thereby converting the halogenated tryptophan to a tryptophan-derived product. Consortia are described in detail above.

In some aspects, the feedstock can include the carbon source in a concentration of at least about 5 g/L (e.g., at least about 10 g/L, at least about 15 g/L, at least about 20 g/L, at least about 25 g/L, at least about 30 g/L, at least about 35 g/L, at least about 40 g/L, at least about 45 g/L, at least about 50 g/L, at least about 55 g/L, at least about 60 g/L, at least about 65 g/L, at least about 70 g/L, at least about 75 g/L, at least about 80 g/L, at least about 85 g/L, at least about 90 g/L, at least about 95 g/L, at least about 100 g/L). In some aspects, the feedstock can include the carbon source in a concentration of up to about 100 g/L (e.g., up to about 95 g/L, up to about 90 g/L, up to about 85 g/L, up to about 80 g/L, up to about 75 g/L, up to about 70 g/L, up to about 65 g/L, up to about 60 g/L, up to about 55 g/L, up to about 50 g/L, up to about 45 g/L, up to about 40 g/L, up to about 35 g/L, up to about 30 g/L, up to about 25 g/L, up to about 20 g/L, up to about 15 g/L, up to about 10 g/L, up to about 5 g/L).

It is considered that the feedstock can include the carbon source in a concentration ranging from any of the minimum values described above to any of the maximum values described above. For example, in some aspects, the feedstock can include the carbon source in a concentration of from about 5 g/L to about 100 g/L (e.g., from about 10 g/L to about 95 g/L, from about 15 g/L to about 90 g/L, from about 20 g/L to about 85 g/L, from about 25 g/L to about 80 g/L, from about 30 g/L to about 75 g/L, from about 35 g/L to about 70 g/L, from about 40 g/L to about 65 g/L, from about 45 g/L to about 60 g/L, from about 50 g/L to about 55 g/L, from about 5 g/L to about 55 g/L, from about 10 g/L to about 50 g/L, from about 15 g/L to about 45 g/L, from about 20 g/L to about 40 g/L, from about 25 g/L to about 35 g/L, from about 50 g/L to about 100 g/L, from about 55 g/L to about 95 g/L, from about 60 g/L to about 90 g/L, from about 65 g/L to about 85 g/L, from about 70 g/L to about 80 g/L).

In some aspects, the feedstock can include tryptophan in a concentration of at least about 1 g/L (e.g., at least about 2 g/L, at least about 3 g/L, at least about 4 g/L, at least about 5 g/L, at least about 6 g/L, at least about 7 g/L, at least about 8 g/L, at least about 9 g/L, at least about 10 g/L, at least about 11 g/L, at least about 12 g/L, at least about 13 g/L, at least about 14 g/L, at least about 15 g/L). In some aspects, the feedstock can include tryptophan in a concentration of up to about 15 g/L (e.g., up to about 14 g/L, up to about 13 g/L, up to about 12 g/L, up to about 11 g/L, up to about 10 g/L, up to about 9 g/L, up to about 8 g/L, up to about 7 g/L, up to about 6 g/L, up to about 5 g/L, up to about 4 g/L, up to about 3 g/L, up to about 2 g/L, up to about 1 g/L).

It is considered that the feedstock can include tryptophan in a concentration ranging from any of the minimum values described above to any of the maximum values described above. For example, in some aspects, the feedstock can include tryptophan in a concentration of from about 1 g/L to about 15 g/L (e.g., from about 2 g/L to about 14 g/L, from about 3 g/L to about 13 g/L, from about 4 g/L to about 12 g/L, from about 5 g/L to about 11 g/L, from about 6 g/L to about 10 g/L, from about 7 g/L to about 9 g/L, from about 1 g/L to about 8 g/L, from about 2 g/L to about 7 g/L, from about 3 g/L to about 6 g/L, from about 4 g/L to about 5 g/L, from about 8 g/L to about 15 g/L, from about 9 g/L to about 14 g/L, from about 10 g/L to about 13 g/L, from about 11 g/L to about 12 g/L).

In some aspects, the at least one upstream engineered microorganism can produce about 100 mg/L or greater (e.g., about 125 mg/L or greater, about 150 mg/L or greater, about 175 mg/L or greater, about 200 mg/L or greater, about 225 mg/L or greater, about 250 mg/L or greater, about 275 mg/L or greater, about 300 mg/L or greater, about 325 mg/L or greater, about 350 mg/L or greater, about 375 mg/L or greater, about 400 mg/L or greater, about 425 mg/L or greater, about 450 mg/L or greater, about 475 mg/L or greater, about 500 mg/L or greater, about 525 mg/L or greater, about 550 mg/L or greater, about 575 mg/L or greater, about 600 mg/L or greater, about 625 mg/L or greater, about 650 mg/L or greater, about 675 mg/L or greater, about 700 mg/L or greater, about 725 mg/L or greater, about 750 mg/L or greater, about 775 mg/L or greater, about 800 mg/L or greater, about 825 mg/L or greater, about 850 mg/L or greater, about 875 mg/L or greater, about 900 mg/L or greater, about 925 mg/L or greater, about 950 mg/L or greater, about 975 mg/L or greater, about 1 g/L or greater) of the halogenated tryptophan.

In some aspects, the at least one downstream engineered microorganism can produce about 100 mg/L or greater (e.g., about 125 mg/L or greater, about 150 mg/L or greater, about 175 mg/L or greater, about 200 mg/L or greater, about 225 mg/L or greater, about 250 mg/L or greater, about 275 mg/L or greater, about 300 mg/L or greater, about 325 mg/L or greater, about 350 mg/L or greater, about 375 mg/L or greater, about 400 mg/L or greater, about 425 mg/L or greater, about 450 mg/L or greater, about 475 mg/L or greater, about 500 mg/L or greater, about 525 mg/L or greater, about 550 mg/L or greater, about 575 mg/L or greater, about 600 mg/L or greater, about 625 mg/L or greater, about 650 mg/L or greater, about 675 mg/L or greater, about 700 mg/L or greater, about 725 mg/L or greater, about 750 mg/L or greater, about 775 mg/L or greater, about 800 mg/L or greater, about 825 mg/L or greater, about 850 mg/L or greater, about 875 mg/L or greater, about 900 mg/L or greater, about 925 mg/L or greater, about 950 mg/L or greater, about 975 mg/L or greater, about 1 g/L or greater) of the tryptophan-derived product.

In another aspect, provided is a tryptophan-derived product generated by any of the disclosed methods.

In some aspects, the tryptophan-derived product can be halogenated. For example, in some aspects, the tryptophan-derived product can include halo-tryptamine, halo-indole-3-acetamide, halo-indole, halo-N-formyl-L-kynurenine, halo-1-acetyl-3-carboxy-β-carboline, halo-2-methyl-L-tryptophan, or any combination thereof.

In other aspects, the tryptophan-derived produced may not halogenated. For example, in some aspects, the tryptophan-derived product can include ryptamine, indole-3-acetamide, indole, N-formyl-L-kynurenine, 1-acetyl-3-carboxy-β-carboline, 2-methyl-L-tryptophan, N-dimethylallyl-L-tryptophan, 4-dimethylallyl-L-tryptophan, 7-dimethylallyl-L-tryptophan, indole-3-acetaldoxime, kynurenine, kynurenic acid, skatole, 3-methyl-2-indolic acid (MIA), serotonin, indole-3-acetic acid (auxin), indole pyruvic acid, strictosidine, indigo, violacein, rebeccamycin, thaxtomin, vinblastine, cyclomarin A, ergoline, ergotarnine, N,N-dimethyltryptarnine, hapalindole A, melatonin, psilocybine, lysergic acid, physostigmine, pyrrolnitrin, quinmerac, or any combination thereof.

In yet other aspects, the tryptophan derived-product can include a combination of halogenated and non-halogenated molecules, for example, any of the halogenated and non-halogenated molecules described above.

In some aspects, the tryptophan-derived product can be a pharmaceutical, a material precursor, a dye, a textile precursor, an agrochemical, a nutraceutical, a flavor, a fragrance, or a food additive.

### EXAMPLES

### Example 1: A Modular and Synthetic Biosynthesis Platform for De Novo Production of Diverse Halogenated Tryptophan-Derived Molecules

In nature, halogenase enzymes generate precisely halogenated end products through a variety of reaction mechanisms [27]. To this end, an array of halogenases have been discovered and can be characterized into four main classes. These include, for example, members of the Fe(II)/alpha KG-dependent class of halogenases such as SyrB2 [28], BesD and similar enzymes [29], and late-stage halogenase WelO5 [30]. Other classes consist of the haloperoxidases and SAM-dependent halogenases [17]. Lastly, flavin-dependent halogenases constitute the final class, including RadH [31] and Rdc2 [32], [33], late-stage halogenase MalA [34], and, of particular interest for this study, the tryptophan halogenases such as RebH, PyrH, and Thal [35][36][37]. Among these enzymes, the tryptophan halogenases have been most extensively studied and engineered over the past few decades mainly in vitro and recently in vivo. More specifically, the varied regioselectivity of these enzymes offers halogenation across the 5-, 6-, or 7-positions of tryptophan with the 7-position most studied in large-scale efforts [27]. The first gram-scale production of 7-chloro-tryptophan from a tryptophan feed was demonstrated in vitro using cross-linked enzyme aggregates (CLEA's) [38]. Recent efforts in the engineering of Corynebacterium glutamicum have demonstrated the first de novo and in vivo gram-scale production of 7-bromo-tryptophan [39]. These studies provide promise for expanding bio-based production to alternative hosts and halogenation positions within tryptophan at a scale relevant for commercially viable industrial production.

The combinatorial biochemistry afforded by linking halogenases with downstream enzymes can access a diverse array of halogenated compounds. Tryptophan itself serves as a gateway to a plethora of interesting natural products ranging from small molecules like indole, kynurenines, quinones, and tryptamines to larger molecules like violacein, strictosidine, and beta-carbolines [40][41][42][43]. To this end, several recent reports describe the in vivo production of tryptophan-derived, halogenated products. For example, halogenated kynurenine derivates, especially 4-chloro-kynurenine, were generated in Streptomyces coeliculr [44], halogenated indolocarbazoles have been generated via combinatorial biosynthesis in S. albus [45], and halogenated tryptophan derivatives for subsequent transition metal catalysis applications [46]. New-to-nature halogenated alkaloids [47], indigoids [48], and auxins [49] have been created in planta. Additionally, halogenated quinolines and alkaloids were realized using yeast bioproduction platforms [50][51]. While these examples demonstrate advances in halogenated metabolism, they do not ubiquitously describe de novo microbial production of diverse halogenated tryptophan-derived compounds starting from simple sugar starting materials. This is an important consideration given the relatively expensive and low-water-soluble substrates often used in prior studies such as indole, tryptophan, or other halogenated precursor molecules, as well as need for de novo production in planta to rely on variable, seasonally-dependent crop yields [51].

This work harnesses halogenases and downstream pathways to generate industrially attractive halogenated molecules in a safe and effective manner through metabolic engineering and synthetic co-cultures. In doing so, this study establishes a co-culture system that uses mix-and-match technology to afford differential downstream halogenation type and position in a manner that enables combinatorial pathway assembly for diverse halogenated molecules. Specifically, the study showcases high-level production of halogenated tryptophan analogs and a collection of strains/pathways that can enable subsequent transformation of these precursors into desirable compounds. Through a synthetic, modular co-culture system, 26 distinct halogenated molecules are generated de novo from glucose, including new-to-nature beta carbolines, prodrug precursors to 4-chloro- and 4-bromo-kynurenine, plant hormone precursors, and other pharmaceutically relevant pre-cursor molecules including tryptamines and indoles. Taken together, this work unlocks halogenated biochemistry by uniting concepts from both combinatorial chemistry and synthetic biology.

### Methods

Chemicals and materials: L-tryptophan, indole, 5-chloroindole, 6-chloroindole, 7-chloroindole, 5-bromoindole, 6-bromoindole and 7-bromoindole were purchased from Sigma-Aldrich. 5-chloro-L-tryptophan, 5-bromo-L-tryptophan, 6-chloro-L-tryptophan, 7-chloro-L-tryptophan, and 7-bromo-L-tryptophan were purchased from Advanced Chemblocks, Inc. 6-bromo-L-tryptophan was purchased from Santa Cruz Biotechnology. D/L versions were purchased from the same vendor. M9, Minimal Salts, 5× was purchased through Sigma-Aldrich.

Strains, plasmids, and transformations: Escherichia coli strain BW25113 with deletions of tnaA or trpR were obtained from E. coli Genetic Resources at Yale CGSC, The Coli Genetic Stock Center. Bacterial genomic DNA was extracted using the Wizard Genomic DNA Purification Kit (Promega). Lamba Red Recombination was used for integration of relevant cassettes (Upstream Module) which has been well-documented elsewhere [90]. pHal, with pBR322 origin and TacI promoter was used for all final experiments. Plasmids were assembled using either Gibson's method, ligation, or a modified Golden Gate cloning method to produce plasmids [91]. Plasmids, primers, ORFs, and other genetic elements are listed in TABLE 1, TABLE 2, and TABLE 3. Various PCR products were then inserted into this plasmid under control of the strong Ptac promoter for overexpression in E. coli. These plasmids are referred to as pHal throughout this work. The inserts were PCR amplified using Q5 Hot Start High-Fidelity DNA Polymerase (NEB). Cells were then electroporated and recovered in 1 mL SOC at 37C for 1 h. A small portion was plated on LB+Amp+Ch1 plates to check for transformation efficiency, while the rest was moved to LB+Amp+Ch1 to grow overnight. Cultures were then freezer stocked and added to screening plates the next day.

TABLE 1
Plasmids used in this study.

| Plasmid | Description | Source | Purpose |
|---|---|---|---|
| pHal | pBR322^{ori}, | This study | Halogenase expression vector |
| | Amp^{R}, | | |
| | P_{Tacl} | | |
| p15A | p15A^{ori}, | This study | Trp Operon (pTrpOp) and |
| | Cm^{R}, | | downstream expression |
| | P₅₀₀ | | enzyme vector |

TABLE 2
Primers used in this study. Similar primers were utilized to amplify
all other listed halogenases with identical Gibson assembly homology.

| Primer name | SEQ ID NO | Purpose |
|---|---|---|
| pHal-iPCR-R | 1 | Amplify pHal backbone |
| pHal-iPCR-F | 2 | Amplify pHal backbone |
| pTacI-ThHalF* | 3 | Amplify ThHal Gibson fragment |
| pTacI-ThHalR* | 4 | Amplify ThHal Gibson fragment |
| pTaci-PyrH-F | 5 | Amplify PyrH Gibson fragment |
| pTaci-PyrHR | 6 | Amplify PyrH Gibson fragment |
| pHal-iPCR-2-R | 7 | Amplify pHal backbone to insert Th- |
| | | Fre |
| pHal-iPCR-2-F | 8 | Amplify pHal backbone to insert Th- |
| | | Fre |
| TrpR::Kan Lambda red primer-F | 9 | Generate TrpR homology fragment for |
| | | LR deletion |
| TrpR::Kan Lambda red primer-R | 10 | Generate TrpR homology fragment for |
| | | LR deletion |
| TnaA::Kan Lambda red primer-F | 11 | Generate TnaA homology fragment |
| | | for LR deletion |
| TnaA::Kan Lambda red primer-R | 12 | Generate TnaA homology fragment |
| | | for LR deletion |
| RbsAR Integration Cassette | 13 | Amplify int. cassette containing AroG, |
| PCR-F | | SerA with RbsAR homology |
| RbsAR Integration Cassette | 14 | Amplify int. cassette containing AroG, |
| PCR-R | | SerA with RbsAR homology |

TABLE 3
Genes, promoters, and other genetic elements used in
this study (generated via PCR or synthesized by IDT).

| | SEQ ID | |
|---|---|---|
| Element name | NO | Purpose |
| P_{TacI} | 15 | Promoter for expression of halogenases |
| Generic Term | 16 | Terminate halogenase expression |
| PyrH | 17 | 5-halo-tryptophan production |
| XsHal | 18 | 5-halo-tryptophan production |
| Thal | 19 | 6-halo-tryptophan production |
| Th-Hal | 20 | 6-halo-tryptophan production |
| SttH | 21 | 6-halo-tryptophan production |
| RebH | 22 | 7-halo-tryptophan production |
| PrnA | 23 | 7-halo-tryptophan production |
| MildUp¹ | 24 | UP element |
| P_{500-LacO}¹ | 25 | Promoter for expression of Th-Fre & all |
| | | downstream enzymes |
| RiboJ¹ | 26 | Genetic insulator |
| RBS7¹ | 27 | RBS |
| rnpB terminator¹ | 28 | Terminator |
| Th-Fre | 29 | Cofactor regeneration |
| EcFre | 30 | Cofactor regeneration (E. coli origin) |
| CymD | 31 | Downstream enzyme expression |
| KynA | 32 | Downstream enzyme expression |
| TsrM | 33 | Downstream enzyme expression |
| TnaA | 34 | Downstream enzyme expression |
| DmaW | 35 | Downstream enzyme expression |
| etpPT | 36 | Downstream enzyme expression |
| RgnTDC | 37 | Downstream enzyme expression |
| iaaM | 38 | Downstream enzyme expression |
| P450 CYP79B2 | 39 | Downstream enzyme expression |
| McbB | 40 | Downstream enzyme expression |
| MildUp² | 24 | UP element |
| P₅₀₀² | 41 | Promoter for expression of TrpOp cassette |
| RiboJ² | 26 | Genetic insulator |
| RBS3² | 42 | RBS |
| TrpE(fbr)DCBA; S40F feedback | 43 | Expression of genes to over- produce of L- |
| resistant mutant highlighted | | Trp |
| P_{J23105}³ | 44 | Expression of upstream module (SerAfbr |
| | | and AroGfbr) |
| B32 rbs³ | 45 | RBS ahead of SerAfbr |
| B34 rbs³ | 46 | RBS ahead of AroGfbr |
| SerA(fbr); H344A, N346A, and | 47 | Expression of SerA(fbr) |
| N364A highlighted | | |
| AroG(fbr) | 48 | Expression of AroG(fbr) |

¹All genetic elements were placed in tandem for Th-Fre and downstream enzyme expression cassettes.
²All genetic elements were placed in tandem for Trp Operon expression cassette (plasmid pTrpOp).
³SerA and AroG integrated into RbsAR as an operon with B34 rbs separating the two coding sequences.

Halogenase and flavin reductase expression: Strains with specified plasmids were typically grown in LB with appropriate antibiotics overnight. The next day, cultures were diluted back to an GD of 0.1 and allowed to grow for 2 h at 37° C. until GD reached 0.7-0.9. 1 mM IPTG was then added and cultures were grown at 30° C. for 2 h to induce expression of halogenase and flavin reductase. Cultures were spun down and media was replaced with M9 salts, 5 g/L glucose, and 1 g/L casamino acids with appropriate antibiotics, 1 mM IPTG, and 1 mM L-tryptophan. Suspension cultures were grown in Fisherbrand™ 96-Well DeepWell™ Polypropylene Microplates and incubated using an Infors HT Multitron Pro with 1000 rpm shaking.

Halogenase panel experimental conditions: Strains containing pHal-ThFre-Hal (various halogenases) were grown in LB with Amp (100 μg/mL) overnight. The next day, cultures were diluted back to an OD of 0.1 and allowed to grow for 2 h at 37° C. until OD reached 0.7-0.9. 1 mM IPTG was then added and cultures were grown at 30° C. for 2 h to induce expression of halogenase and flavin reductase. Cultures were spun down and media was replaced with M9G+CAA with appropriate antibiotics, 1 mM IPTG, and 1 mM L-tryptophan. Suspension cultures were grown in Fisherbrand™ 96-Well DeepWell™ Polypropylene Microplates and incubated using an Infors HT Multitron Pro with 1000 rpm shaking.

De novo production of halogenated tryptophan in E. coli: Strains containing specified modifications were grown up in LB with any necessary antibiotics overnight at 30° C. The next day, these strains were diluted back 100-fold to OD ˜0.1 and allowed to grow to OD 0.7-0.9 in 25 mL LB media at 37° C. in a 250 mL shake flask. Cultures were then induced with 1 mM IPTG and allowed to grow for 2 h at 30° C. Cultures were then spun down at 3000×g for 10 min in Falcon tubes to remove LB media and were replaced with 25 mL of M9G media (M9 salts, 40 g/L glucose) and placed in a 250 mL shake flask. These were then allowed to grow up for 72 h with timepoints taken every 12 h to be analyzed on HPLC.

Downstream promiscuity feeding assays: Strains containing corresponding downstream enzymes were grown up in LB+Chl (34 μg/mL) overnight. Strains were then inoculated at OD ˜0.1 in M9G+CAA+Chl and allowed to grow for 2 h in which 1 mM IPTG and 500 μM of corresponding halogenated tryptophan analogs were added to the media. Downstream products were then confirmed via LC-MS.

Downstream promiscuity docking studies: The Rosetta software suite is a platform for the computational modeling of protein structures. PyRosetta, a Python binding for Rosetta [92], was utilized to compare the structures of iaaM and McbB (Protein Data Bank accession codes of 4iv9 and 3×27, respectively), as these crystal structures are the only structures in this study to contain bound tryptophan. An ensemble of conformations in “.mol” format were generated for 5-chloro-, 5-bromo-, 6-chloro-, 6-bromo-, 7-chloro-, and 7-bromo-tryptophan using the OpenBabel chemical toolbox. These were then converted into a “.param“file and several”.pdb” files for use with PyRosetta. Each halogenated tryptophan was aligned to the native tryptophan binding mode in each enzyme crystal structure, resulting in 14 complexes including the original complex. Each complex was minimized into Rosetta's energy scoring system using the “ref2015_cart” scorefunction and FastRelax. Finally, each complex was analyzed using the InterfaceAnalyzerMover to determine an overall binding energy or AAG.Allcodeusedtogeneratethe structures and binding scores can be found at https://github.com/jordantwells42/downstream-docking.

De novo co-culture production of halogenated tryptophan derivatives in E. coli: Strains containing specified modifications were grown up in LB with any necessary antibiotics overnight at 30° C. The next day, these strains were diluted back 100-fold to OD˜0.1 and allowed to grow to OD 0.7-0.9 in 1 mL LB media at 37° C. Cultures were then induced with 1 mM IPTG and allowed to grow for 2 h at 30° C. Cultures were then spun down at 3000×g for 10 min to remove LB media and were replaced with 500 μL of M9G media with micronutrients (M9 salts, 40 g/L glucose, 1× micronutrient solution), then combined together in a single reaction of 1 mL total volume. These were then allowed to incubate for 48 h, where the supernatants were spun down and analyzed on HPLC.

Quantification of products: Cultures were typically grown at 30° C. in a shaking incubator at 1000 rpm for specified amounts of time (0-48 h). After specified amounts of time, OD600 was measured using a Tecan plate reader as necessary. Cultures were then centrifuged at 3000×g for 10 min to pellet the cells, and the supernatant was removed for further analysis. Metabolite quantification was performed on HPLC or LC-MS using authentic standards, depending on availability. Supernatants were then submitted for LC-MS analysis to confirm the presence of the expected downstream products for each reaction or compared to authentic standards on HPLC for the case of indole analogs, which had difficulty fragmenting on the MS, even at high concentrations of an authentic standard. Quantification was performed on a Dionex Ulti-Mate 3000 (Thermo) equipped with an LS Eclipse Plus C18 column (3.0×150 mm, 3.5 μm; Agilent). The mobile phase for tryptophan and halogenated tryptophan analysis consisted of 1% (v/v) acetic acid in water or acetonitrile. Detection was performed at 280 nm with a flow rate of 0.3 mL min⁻¹ and a column temperature of 30° C. Data processing was performed using Chromeleon software. Calibration standards were prepared for tryptophan and halogenated tryptophan. Downstream molecules were detected on an LC-MS. Sample supernatants were loaded directed into the instrument without additional preparation. All measurements were performed in biological triplicate with representative spectra displayed in the supplemental information. For LC/MS analysis, the samples were analyzed using an Agilent 6546 A Q-TOF interfaced with an Agilent 1260 Infinity II liquid chromatography system (G7112B) and an Agilent Dual Jet Stream electro-spray ionization (ESI) source (G1958-65271). The mass spectrometry conditions were as follows: autosampler temperature 7° C.; column temperature 30° C.; electrospray ionization in positive mode; capillary voltage 3500 V; nozzle voltage 2000 V; fragmentor voltage 80 V; nitrogen drying gas temperature 350° C.; nitrogen drying gas flow rate 10 L/min; sheath gas temperature 350° C.; sheath gas flow rate 11 L/min; nebulizer pressure 60 psi; mass range 50-1000 m/z. LC separations were achieved on an Agilent Rapid Resolution HD ZORBAX Eclipse Plus C18 column (P.N. 959757-902: 50×2.1 mm, 1.8 micron particle size) preceded by an Agilent ZORBAX Eclipse Plus C18 narrow bore guard column (P.N. 821125-936: 12.5×2.1 mm, 5 micron particle size). The LC conditions were as follows: solvent A was Water with 0.1% formic acid; solvent B was Acetonitrile; flow rate 0.4 mL/min; gradient ramp held 5% B for 2 min, ramped to 20% B from 2 to 5 min, ramped to 95% B from 5 to 12 min, held at 95% B until 16 min, then re-equilibrated at 5% B from 16.1 to 20 min. LC/MS data were collected using Agilent MassHunter Workstation LC/MS Data Acquisition for 6500 series Q-TOF (Version 10.1) and analyzed using Agilent MassHunter Workstation Qualitative Analysis (Version 10.0) software. All m/z values and spectra were calculated and collected based on the expected structures of the respective compounds of interest using MassHunter's internal search function.

### Results

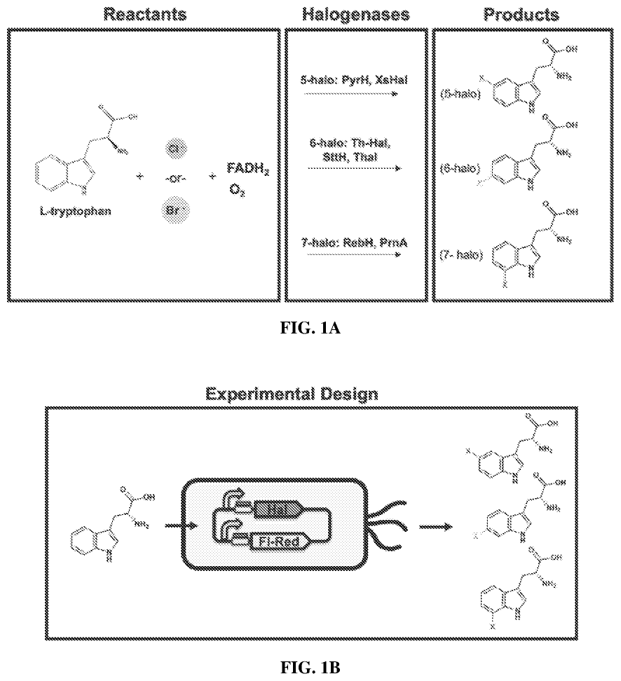

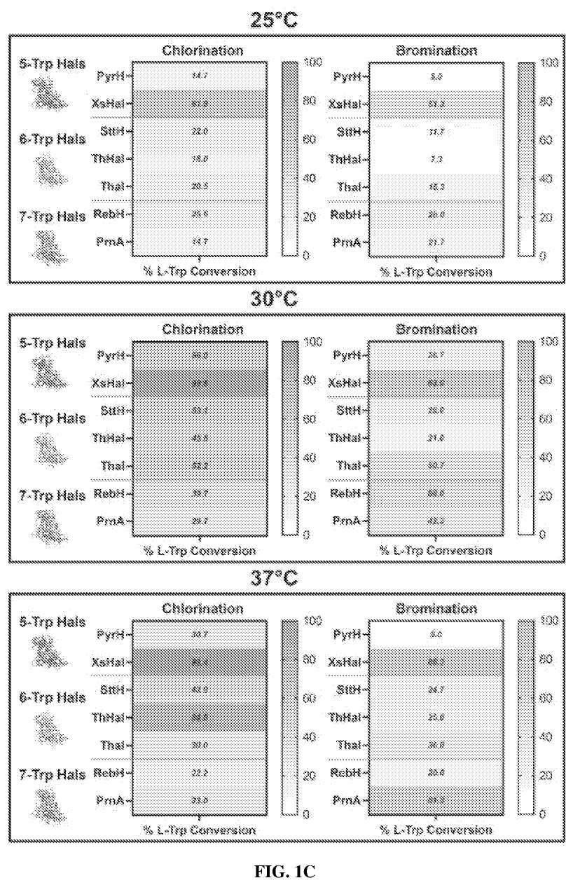

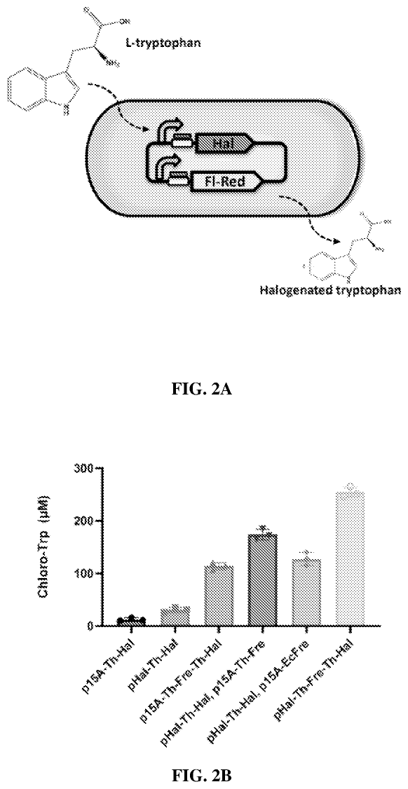

Enabling halogenated tryptophan production in an E. coli platform: While de novo production of halogenated tryptophan has been reported in C. glutamicum for 7-Br-tryptophan and detectable quantities of 7-Cl-tryptophan [39][52], no studies have reported such production (for these particular halogenated forms or others) of close to gram-scale titers in E. coli. Initial efforts here evaluated the synthetic expression of halogenases and flavin reductase cofactor rebalance partners to enable production (FIGS. 1A-1C). Specifically, production of halogenated tryptophan was evaluated using varying expression regimes for Th-Hal and flavin reductases Th-Fre and EcFre (FIGS. 2A-2B). These results reinforce previous literature that the native E. coli flavin reductase (EcFre) is not expressed highly enough to enable sufficient cofactor rebalance of FAD to FADH₂ for high-level production of halo-tryptophan using tryptophan halogenases [53]. Specifically, only minimal halogenated tryptophan (e.g., 40 μM of 6-chloro-tryptophan from 1 mM of tryptophan fed) was produced without overexpression of a flavin reductase (FIGS. 2A-2B). Expression of a heterologous, more thermostable flavin reductase (Th-Fre) resulted in higher production over EcFre and the null strain, reinforcing the importance of optimizing the cofactor balance for the halogenase reaction as noted in prior studies [36][37][38][46][47]. This system also allowed for the optimization of copy number and promoters driving expression of Th-Hal as a model halogenase.

Development of optimized halogenase expression strategy: The study explored production of halogenated tryptophan with different copy number plasmids and promoters using Th-Hal as a model halogenase. Th-Hal was either expressed under an inducible version of promoter J23101 on plasmid p15A or under an inducible Tac promoter with origin pBR322, denoted as pHal. It was discovered that expression on plasmid pHal led to the production of around 1.6-fold as much halogenated tryptophan compared to expression on plasmid p15A, reaching nearly 250 M after 24 hours. Since de novo production from glucose has not been realized for all possible flavors of halogenated tryptophan, the study proceeded forward with the best expression strategy developed herein.

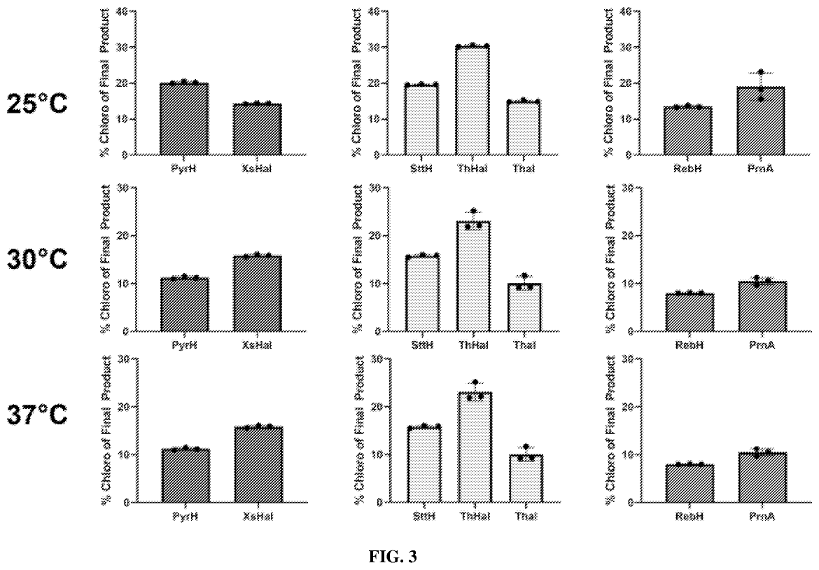

Establishing a panel of halo-tryptophan precursor production through halogenase selection: Using the expression platform above, the study next evaluated the in vivo halogenation profile for a collection of halogenases comprised of at least two homologues capable of catalyzing each regioselective reaction at the 5, 6, or 7 positions of tryptophan including well-studied halogenases like Thal [36], RebH [37], PrnA [54], and PyrH [35](TABLE 4). Tryptophan feeding assays were conducted (FIG. 1C) at varying temperatures to investigate the robustness of each enzyme and evaluation of chloro- and bromo-preferences (FIG. 3).

TABLE 4
List of halogenase enzymes used in this study.

| | | | Halogenated |
|---|---|---|---|
| Halogenase | Organism of origin | Product formed | position |
| PyrH | Streptomyces rugosporus | 5-halo- | 5 |
| | | tryptophan | |
| XszenFHal | Xenorhabdus szentirmaii | 5-halo- | 5 |
| (XsHal) | | tryptophan | |
| Thal | Streptomyces albogriseolus | 6-halo- | 6 |
| | | tryptophan | |
| Th-Hal | Streptomyces | 6-halo- | 6 |
| | violaceusniger | tryptophan | |
| SttH | Streptomyces toxytricini | 6-halo- | 6 |
| | | tryptophan | |
| RebH | Lentzea aerocolonigenes | 7-halo- | 7 |
| | | tryptophan | |
| PrnA | Pseudomonas fluorescens | 7-halo- | 7 |
| | | tryptophan | |

A few initial observations can be made from the halogenase panel. First, the halogenase XsHal displayed robust in vivo production of both 5-chloro- and 5-bromo-tryptophan precursors. At all temperatures, XsHal performs significantly better than its counterpart PyrH, an enzyme that has a very low reported melting temperature of around 30° C. [55]. Furthermore, XsHal is reported to have a 2-fold increase in catalytic efficiency over PyrH [56] in vitro, yet exhibits multi-fold higher production in vivo at various temperatures. Second, the halogenase Thal shows the most consistent generation of both 6-chloro- and 6-bromo-tryptophan precursors at 30° C., whereas Th-Hal, a reported halogenase from a thermophilic organism, shows the highest conversion of the tryptophan to 6-halo-tryptophan at 37° C., with an evident preference for chloro- over bromo-addition. For the 7-tryptophan halogenases, RebH, which was previously used for other de novo halogenated molecule production in planta [47], provided the most consistent conversion of tryptophan into 7-bromo- and 7-chloro-tryptophan precursors at 30° C., whereas PrnA could be used as a reliable halogenase at higher temperatures. Lastly, halogenation was almost universally restricted at 25° C. with the exception of XsHal that enabled high turnover even at this suboptimal operating temperature. Based on these results, halogenases XsHal, Thal, and RebH were selected based on their superior conversion at 30° C. and ability to collectively access multiple halogenation sites for both chlorine and bromine on tryptophan.

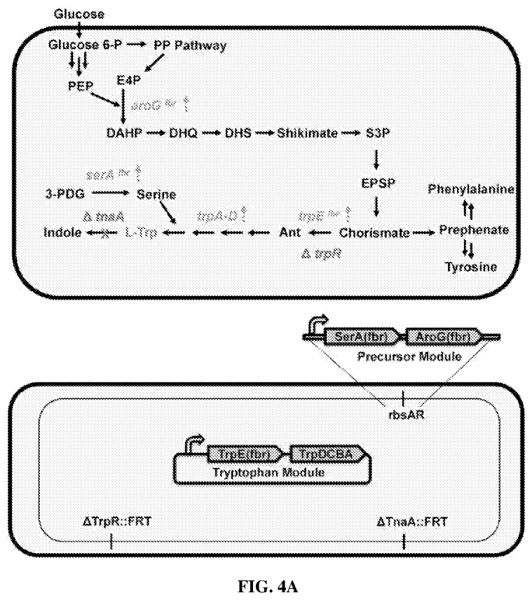

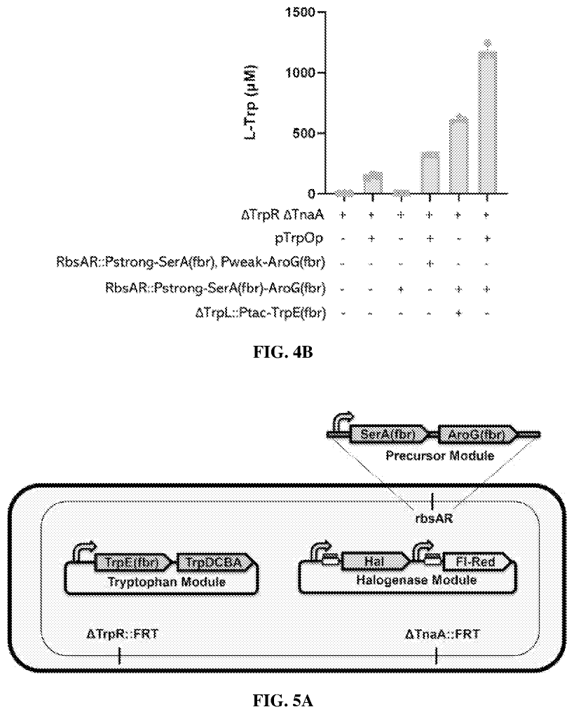

Enabling de novo production of halo-tryptophan precursors through metabolic engineering: After characterizing the potential of E. coli to express functional halogenases and selecting a collection of functional enzymes, the study employed a metabolic engineering approach to improve precursor availability and boost halo-tryptophan precursor production de novo. This effort focused broadly on the three goals of: (i) removal of degradation mechanisms, (ii) removal of feedback regulation, and (iii) overexpression of biosynthetic pathway enzymes (FIG. 4A).

First, degradation was removed through targeting the tryptophanase (encoded by tnaA) for deletion to remove degradation into indoles [57] and deletion of the TrpR transcriptional repressor that serves to regulate biosynthesis and transport [58]. Disruption of the trpR and tnaA genes did not immediately show appreciable accumulation of tryptophan in the cellular supernatant after 24 h (FIG. 4B). Second, removal of feedback inhibition and overexpression of biosynthetic enzymes in the aromatic amino acid pathway were incorporated into the strain and leveraged efforts of many reports to improve tryptophan overproduction [59][60][61] including canonical targets including mutations in TrpE, AroG, and SerA to enable feedback resistance (fbr) [62]. A synthetic modularization of metabolism approach [63] was conducted here consisting of a Precursor Module and a Tryptophan Biosynthesis Module. In this case, the Precursor Module consisted of over-expressions of AroG(fbr) and SerA(fbr) and the Tryptophan Bio-synthesis Module consists of all the genes in the trp operon with peptide leader TrpL removed and the feedback-resistant mutant of TrpE, TrpE(fbr). Expression strength and copy numbers of these modules were optimized to obtain high levels of tryptophan over-production (FIG. 4B). TABLE 5 outlines the collective metabolic modifications investigated in this study. The final strain modification strategy resulted in a strain (E. coli sKR-Trp4) capable of producing over 200 mg/L of tryptophan after 24 h in minimal media containing only 5 g/L glucose in a 96-deep well plate (1 mL scale) (FIG. 4B).

TABLE 5
Tryptophan overproduction strains with corresponding
modifications constructed in this study.

| Strain | |
|---|---|
| name | Modifications |
| SKR-Trp0 | ΔTnaA::FRT |
| SKR-Trp1 | ΔTrpR::FRT and ΔTnaA::FRT |
| SKR-Trp2 | ΔTrpR::FRT, ΔTnaA::FRT, and pTrpOp-TrpE(fbr)DCBA |
| SKR-Trp3 | ΔTrpR::FRT, ΔTnaA::FRT, |
| | pTrpOp-TrpE(fbr)DCBA, and rbsAR::pJ23105- |
| | B32-SerA(fbr)-Pyibn-AroG(fbr) |
| SKR-Trp4 | ΔTrpR::FRT, ΔTnaA::FRT, |
| | pTrpOp-TrpE(fbr)DCBA, and rbsAR::pJ23105- |
| | B32-SerA(fbr)-B34-AroG(fbr) |
| SKR-Trp5 | ΔTrpR::FRT, ΔTnaA::FRT, |
| | ArbsAR::pJ23105-B32-SerA(fbr)-B34-AroG(fbr), |
| | and ΔtrpL::TP24-Ptac-TrpE(fbr)-TrpDCBA |

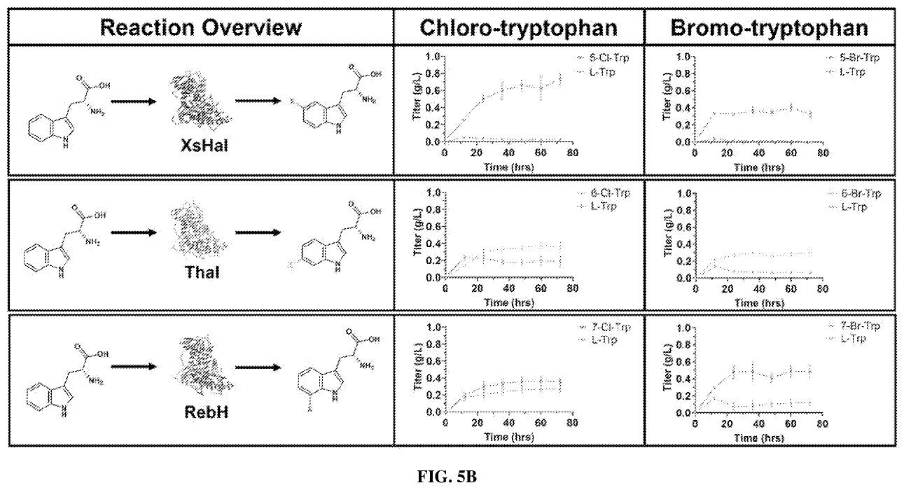

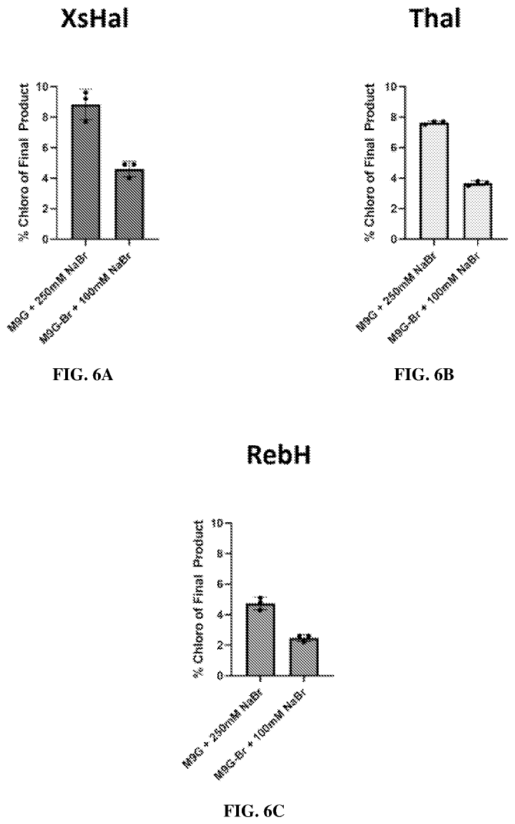

Using this high-tryptophan producing E. coli strain, it is possible to incorporate the above characterized halogenases to enable de novo production of halo-tryptophan precursors. To do so, the sKR-Trp4 strain was transformed with either XsHal, Thal, and RebH to generate three strains respectively (sKR-Trp4-XsHal, sKR-Trp4-Thal, and sKR-Trp4-RebH) (FIG. 5A). The resulting de novo titers of 0.3-0.7 g/L halogenated tryptophan for each corresponding halogenase fed with 40 g/L glucose and the corresponding halide salt are shown in FIG. 5B. These strains achieve a selectivity reaching as high as 96% for bromo-tryptophan even in the presence of competition for the halide salt with the residual chloride present in the growth media (FIGS. 6A-6C), consistent with previously reported selectivity in other organisms that use similar levels of bromide salt in the media [39]. Altogether, these findings showcase the highest titers of de novo varied halogenated tryptophan production at flask scale to date. It is important to note that these products are secreted and thus enable an easy access point for creating a modular diversification approach using a co-culture.

Removal of feedback inhibition to bolster tryptophan titer: Removal of feedback inhibition in three particular biosynthesis proteins has been shown to have the largest impact on tryptophan overproduction in a variety of studies [49][50][51]. These include proteins TrpE, AroG, and SerA, where removal of feedback inhibition is well documented [52]. A linear integration cassette was constructed to enable strong expression of SerA(fbr) and either weak or strong expression of AroG(fbr) to assess the need for general expression optimization. TrpE(fbr) was expressed with a medium strength promoter on a medium copy number plasmid (pTrpOp). In addition, an integration cassette was constructed to replace the TrpL gene locus with a strong, constitutive, unregulated promoter and TrpE(fbr), thus generating a similar overexpression cassette for the trp operon in the genome to compare to the plasmid-borne approach. It was found that the highest levels of tryptophan were generated when AroG(fbr) was strongly expressed and the trp operon was expressed on a plasmid with a medium-strength promoter. Interestingly, additive overexpressions of the tryptophan biosynthesis genes (e.g., expression using both the pTrpOp plasmid and the TrpL::Ptac trp operon cassette) yielded a significant growth deficit, corroborating results from previous tryptophan overproduction endeavors, both empirical and computational [54][55].

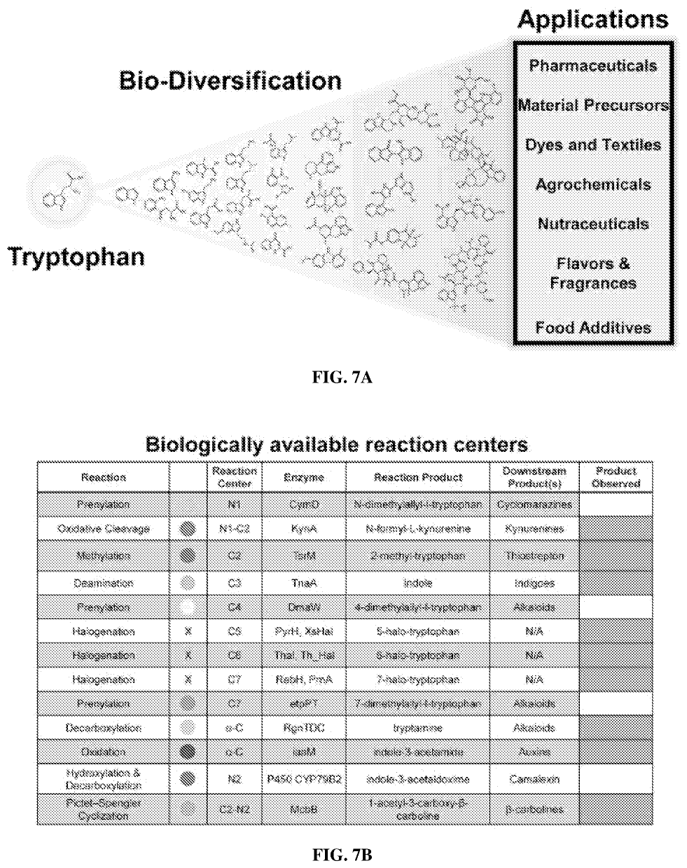

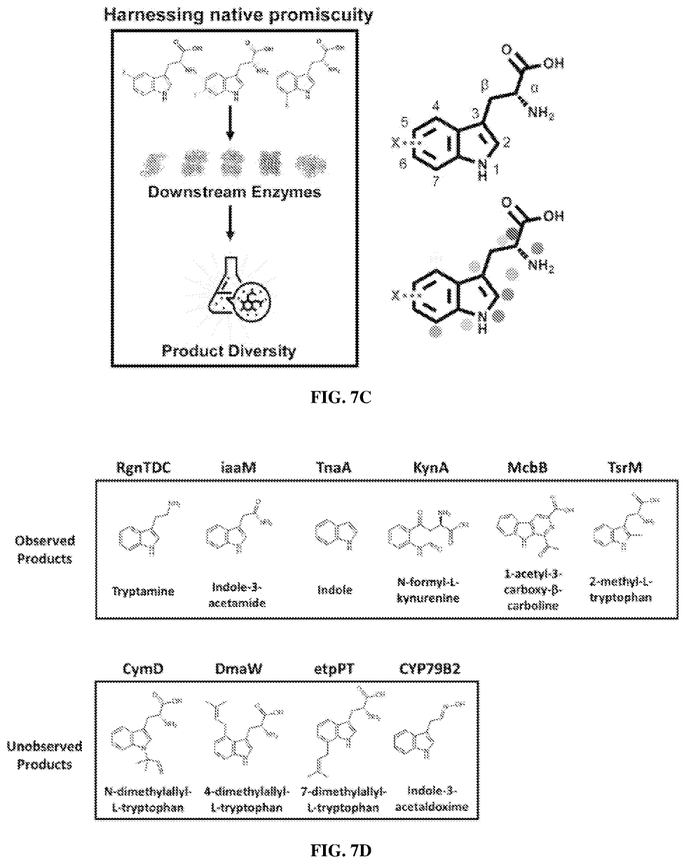

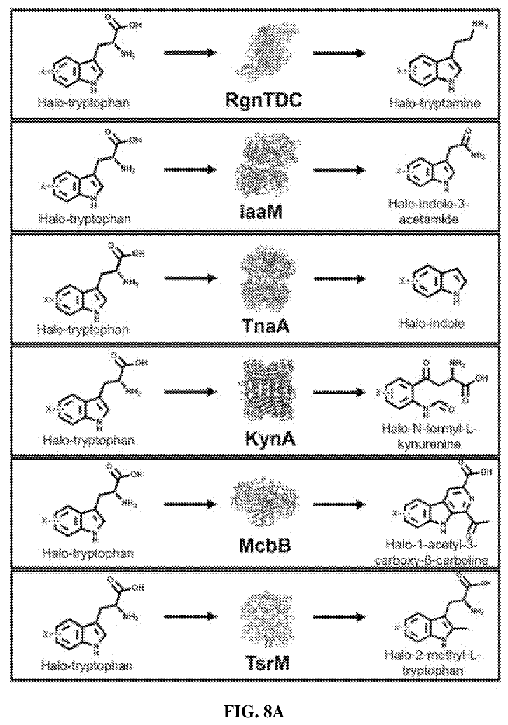

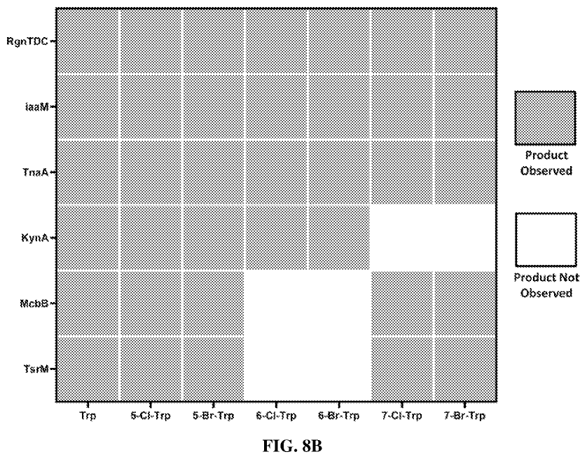

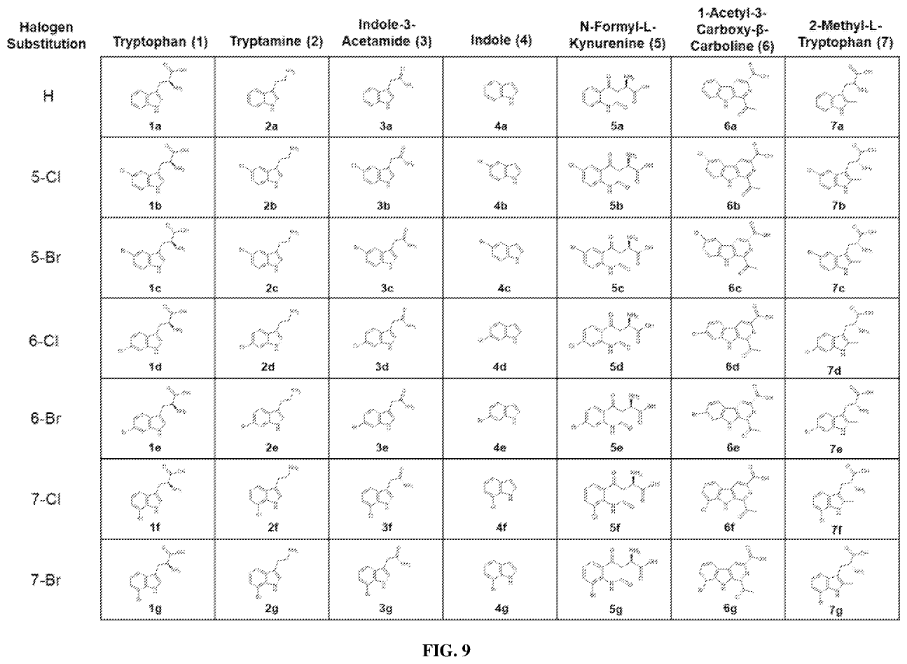

Initial halogen-product diversification through complementing promiscuous enzymes and feeding assays: Tryptophan is the most chemically complex proteogenic amino acid and can quickly be modified in many positions through even just single enzymatic steps to yield products with a diverse range of applications (FIG. 7A). To enable an exploration of this chemical space with halogenated compounds, enzymes were selected to target as many biologically accessible reaction centers of tryptophan as possible (FIGS. 7A-7D, as determined by Transform MinER online module [64]). A total of 10 modifying enzymes (beyond the halogenases described earlier) were selected and evaluated for their ability to convert 500 M of fed L-tryptophan to a corresponding downstream product. Of the explored set of enzymes, five of these enzymes (encoded by RgnTDC, iaaM, TnaA, KynA, and McbB) enabled conversion of all fed tryptophan (TABLE 6, TABLE 7, and TABLE 8). Molecules corresponding to the theoretical product 2a, 3a, 4a, 5a, and 6a were observed for each enzyme, respectively (FIG. 8A). Beyond this set, TsrM was observed to catalyze the reaction of tryptophan to 2-methyl-L-tryptophan (7a), though showed very minimal conversion of tryptophan fed and yielded low intensities on the LC-MS samples. Other enzymes did not convert any appreciable tryptophan under various fermentation and expression conditions after 48 h and the corresponding theoretical products 8a, 9a, 10a, and 11a were not observed (FIG. 5B; shown in white in the “Product Observed” column). These enzymes comprise either prenyltransferases, including CymD [65], DmaW [66], and etpPT [67], and thus require a large pool of DMAPP to function well in vivo or are P450s, such as CYP79B2, with documented difficulties in soluble expression. Nonetheless, a wide range of reactions are represented in this functional subset including a ring-opening, a ring-closing, cleavage of the amino acid group, and multiple modifications on the amino acid portion of tryptophan. Specifically, RgnTDC is a tryptophan decarboxylase (TDC) from the organism Ruminococcus gnavus, and catalyzes the formation of tryptamine, a physiologically important and highly relevant pharmaceutical precursor [68][69]. RgnTDC was previously characterized to be highly promiscuous towards many tryptophan derivatives, including halogenated ones [70]. iaaM is an enzyme involved in the production of auxin in plants, catalyzing the generation of indole-3-acetamide, and characterized to be promiscuous as well, although unknown at the outset of this study [71]. TnaA, E. coli's native tryptophan indole lyase, catalyzes the production of indole, a pre-cursor for many other molecules including indigo and other important tryptophan dimers [72][73]. KynA catalyzes a ring-opening reaction to generate N-formyl-L-kynurenine and comprises the first step in the kynurenine and quinone pathways, classes of molecules with many bioactive characteristics [50][74][75]. McbB, one of the genes that drives the biosynthesis of marinacarbolines, was found in the organism Marinactinospora thermotolerans SCSIO 00652, and catalyzes a Pictet-Spengler cyclization process [76]. Beta-carbolines in general have been shown to have very interesting chemical characteristics including optoelectronic properties, potential as anti-cancer agents, and many other bioactivities [77][78][79][80]. Specifically, the molecules formed through the Mcb pathway in Marinactinospora thermotolerans have been shown to have antimalarial, cytotoxic, and anti-inflammatory activities [81][82]. TsrM catalyzes the reaction to 2-methyl-L-tryptophan via a unique cobalamin-dependent radical SAM mechanism and is the first step towards the synthesis of the antibiotic thiostrepton A [83][84][85]. Thus, this set showcases a wide range of biochemical reactions with fundamentally valuable end products.

TABLE 6
Estimated titers of downstream molecules produced by both the feeding assays
and cocultures. As standards were not available for the vast majority of produced
downstream molecules, titers from feeding assays (FIGS. 8A-8B) were estimated
based on consumed tryptophan or halogenated tryptophan precursor fed (on a mM
basis), assuming all consumed precursor was converted to the desired downstream
product. For the cocultures, estimated titers were calculated by multiplying
the feeding assay estimated titers by the relative LCMS abundances of the appropriate
downstream product (area from coculture divided by that of feeding assay). Direct
comparisons of LCMS abundances were only carried out when values were from the
same downstream product, to avoid the impact of differing ionization capacity
impacting the results. Compounds not detected are denoted as N.D. Feeding assay
approximate titer was estimated based on consumed halo-trp precursor, assuming
all precursor consumed was converted to product. Coculture approximate titer
was determined by the ratio of LCMS abundance for coculture/feeding assay multiplied
by feeding assay approximate titer.

| | | | Feeding Assay | Coculture |
|---|---|---|---|---|
| Downstream | Halo | | Approx Titer | Approx Titer |
| Enzyme | Position | Product | [mM]* | [mM]** |
| TDC | n/a | Tryptamine | 0.5 | 0.55 |
| TDC | 5 Cl | 5-Cl-Tryptamine | 0.43 | 0.3 |
| TDC | 5 Br | 5-Br-Tryptamine | 0.46 | 0.21 |
| TDC | 6Cl | 6-Cl-Tryptamine | 0.5 | 0.13 |
| TDC | 6Br | 6-Br-Tryptamine | 0.32 | 0.22 |
| TDC | 7Cl | 7-Cl-Tryptamine | 0.5 | 0.16 |
| TDC | 7Br | 7-Br-Tryptamine | 0.34 | 0.31 |
| TnaA | n/a | Indole | 0.5 | 0.9 |
| TnaA | 5 Cl | 5-Cl-Indole | 0.45 | 1.09 |
| TnaA | 5 Br | 5-Br-Indole | 0.33 | 1.04 |
| TnaA | 6Cl | 6-Cl-Indole | 0.5 | 0.64 |
| TnaA | 6Br | 6-Br-Indole | 0.27 | 0.83 |
| TnaA | 7Cl | 7-Cl-Indole | 0.5 | 0.12 |
| TnaA | 7Br | 7-Br-Indole | 0.32 | 0.22 |
| IaaM | n/a | Indole-3-Acetamide | 0.5 | 3.76 |
| IaaM | 5 Cl | 5-Cl-Indole-3- | 0.41 | 0.75 |
| | | Acetamide | | |
| IaaM | 5 Br | 5-Br-Indole-3- | 0.31 | 0.5 |
| | | Acetamide | | |
| IaaM | 6Cl | 6-Cl-Indole-3- | 0.5 | 0.2 |
| | | Acetamide | | |
| IaaM | 6Br | 6-Br-Indole-3- | 0.3 | 0.3 |
| | | Acetamide | | |
| IaaM | 7Cl | 7-Cl-Indole-3- | 0.5 | 0.29 |
| | | Acetamide | | |
| IaaM | 7Br | 7-Br-Indole-3- | 0.38 | 0.71 |
| | | Acetamide | | |
| KynA | n/a | N-Formyl-L-Kynurenine | 0.5 | 0.64 |
| KynA | 5 Cl | 5-Cl-N-Formyl-L- | 0.1 | 0.06 |
| | | Kynurenine | | |
| KynA | 5 Br | 5-Br-N-Formyl-L- | 0.02 | 0.03 |
| | | Kynurenine | | |
| KynA | 6Cl | 6-Cl-N-Formyl-L- | 0.1 | 0.098 |
| | | Kynurenine | | |
| KynA | 6Br | 6-Br-N-Formyl-L- | 0.01 | 0.05 |
| | | Kynurenine | | |
| KynA | 7Cl | 7-Cl-N-Formyl-L- | N.D. | N.D. |
| | | Kynurenine | | |
| KynA | 7Br | 7-Br-N-Formyl-L- | N.D. | N.D. |
| | | Kynurenine | | |
| McbB | n/a | 1-acetyl-3-carboxy-B- | 0.5 | 0.13 |
| | | carboline | | |
| McbB | 5 Cl | 5-Cl-1-acetyl-3-carboxy- | 0.13 | 0.03 |
| | | B-carboline | | |
| McbB | 5 Br | 5-Br-1-acetyl-3- | 0.01 | 0.02 |
| | | carboxy-B-carboline | | |
| McbB | 6Cl | 6-Cl-1-acetyl-3-carboxy- | N.D. | N.D. |
| | | B-carboline | | |
| McbB | 6Br | 6-Br-1-acetyl-3- | N.D. | N.D. |
| | | carboxy-B-carboline | | |
| McbB | 7Cl | 7-Cl-1-acetyl-3-carboxy- | 0.12 | 0.0015 |
| | | B-carboline | | |
| McbB | 7Br | 7-Br-1-acetyl-3- | 0.08 | 0.0024 |
| | | carboxy-B-carboline | | |
| TsrM | n/a | 2-methyl-L-tryptophan | 0.08 | N.D. |
| TsrM | 5 Cl | 5-Cl-2-methyl-L- | 0.04 | N.D. |
| | | tryptophan | | |
| TsrM | 5 Br | 5-Br-2-methyl-L- | 0.08 | N.D. |
| | | tryptophan | | |
| TsrM | 6Cl | 6-Cl-2-methyl-L- | N.D. | N.D. |
| | | tryptophan | | |
| TsrM | 6Br | 6-Br-2-methyl-L- | N.D. | N.D. |
| | | tryptophan | | |
| TsrM | 7Cl | 7-Cl-2-methyl-L- | 0.03 | N.D. |
| | | tryptophan | | |
| TsrM | 7Br | 7-Br-2-methyl-L- | 0.02 | N.D. |
| | | tryptophan | | |

TABLE 7
Titer benchmarking for halo-tryptamine production. Benchmarking
estimated coculture titers from TABLE 6 against commercially
available analytical standards for 5-chloro-tryptamine and
5-bromo-tryptamine. Exact titers were determined via HPLC
via comparison with an analytical standard and are graphically
represented in FIG. 13. Estimated titers were determined
via the method described in TABLE 6.

| | Exact titer via analytical | Estimated titer from |
|---|---|---|
| Product | standard (mg/L) | TABLE 6 (mg/L) |
| 5-Cl-Tryptamine | 38.7 ± 1.4 | 58.4 |
| 5-Br-Tryptamine | 51.7 ± 6.4 | 50.2 |

TABLE 8
Estimated conversion of fed tryptophan or halo-tryptophan precursor in feeding
assays. Conversion values calculated by dividing estimated feeding assay titers
from TABLE 7 by the quantity of precursor fed, multiplied by 100%. Feeding
assay estimated conversion was estimated based on consumption of 0.5 mM fed
precursor, assuming all precursor consumed was converted to product.

| Product | Feeding Assay Estimated Conversion [%] |
|---|---|
| Tryptamine | 100 |
| 5-Cl-Tryptamine | 86 |
| 5-Br-Tryptamine | 92 |
| 6-Cl-Tryptamine | 100 |
| 6-Br-Tryptamine | 64 |
| 7-Cl-Tryptamine | 100 |
| 7-Br-Tryptamine | 68 |
| Indole | 100 |
| 5-Cl-Indole | 90 |
| 5-Br-Indole | 66 |
| 6-Cl-Indole | 100 |
| 6-Br-Indole | 54 |
| 7-Cl-Indole | 100 |
| 7-Br-Indole | 64 |
| Indole-3-Acetamide | 100 |
| 5-Cl-Indole-3-Acetamide | 82 |
| 5-Br-Indole-3-Acetamide | 62 |
| 6-Cl-Indole-3-Acetamide | 100 |
| 6-Br-Indole-3-Acetamide | 60 |
| 7-Cl-Indole-3-Acetamide | 100 |
| 7-Br-Indole-3-Acetamide | 76 |
| N-Formyl-L-Kynurenine | 100 |
| 5-Cl-N-Formyl-L-Kynurenine | 20 |
| 5-Br-N-Formyl-L-Kynurenine | 4 |
| 6-Cl-N-Formyl-L-Kynurenine | 20 |
| 6-Br-N-Formyl-L-Kynurenine | 2 |
| 7-Cl-N-Formyl-L-Kynurenine | 0 |
| 7-Br-N-Formyl-L-Kynurenine | 0 |
| 1-acetyl-3-carboxy-B-carboline | 100 |
| 5-Cl-1-acetyl-3-carboxy-B-carboline | 26 |
| 5-Br-1-acetyl-3-carboxy-B-carboline | 2 |
| 6-Cl-1-acetyl-3-carboxy-B-carboline | 0 |
| 6-Br-1-acetyl-3-carboxy-B-carboline | 0 |
| 7-Cl-1-acetyl-3-carboxy-B-carboline | 24 |
| 7-Br-1-acetyl-3-carboxy-B-carboline | 16 |
| 2-methyl-L-tryptophan | 16 |
| 5-Cl-2-methyl-L-tryptophan | 8 |
| 5-Br-2-methyl-L-tryptophan | 16 |
| 6-Cl-2-methyl-L-tryptophan | 0 |
| 6-Br-2-methyl-L-tryptophan | 0 |
| 7-Cl-2-methyl-L-tryptophan | 6 |
| 7-Br-2-methyl-L-tryptophan | 4 |

Next, the study sought to evaluate and harness the promiscuity of these downstream enzymes toward fed halogenated substrates in an effort to create diverse halogenated products. As outlined in FIG. 8A, the six selected downstream enzymes were each evaluated for their ability to convert six different halogenated tryptophan variants in the background of a tnaA deletion strain to prevent any unwanted product degradation. The binary promiscuity observations for these 42 combinations (when considering tryptophan and all 6 halogenated variants) are provided in FIG. 8B, whereas structures are displayed in FIG. 9. From these assays, all 6 potential molecule sets 2b, 2c, 2d, 2e, 2f, 2g and 3b, 3c, 3d, 3e, 3f, 3g were observed when RgnTDC and iaaM were expressed, respectively. RgnTDC and iaaM are thus remarkably promiscuous enzymes, evidenced by their ability to convert all positions of halogenated tryptophan, supported by similar reports in prior literature [70][71]. All possible molecules 4b, 4c, 4d, 4e, 4f, and 4g were also observed when TnaA was expressed, thus echoing similar trends of promiscuity reports from this enzyme [53].

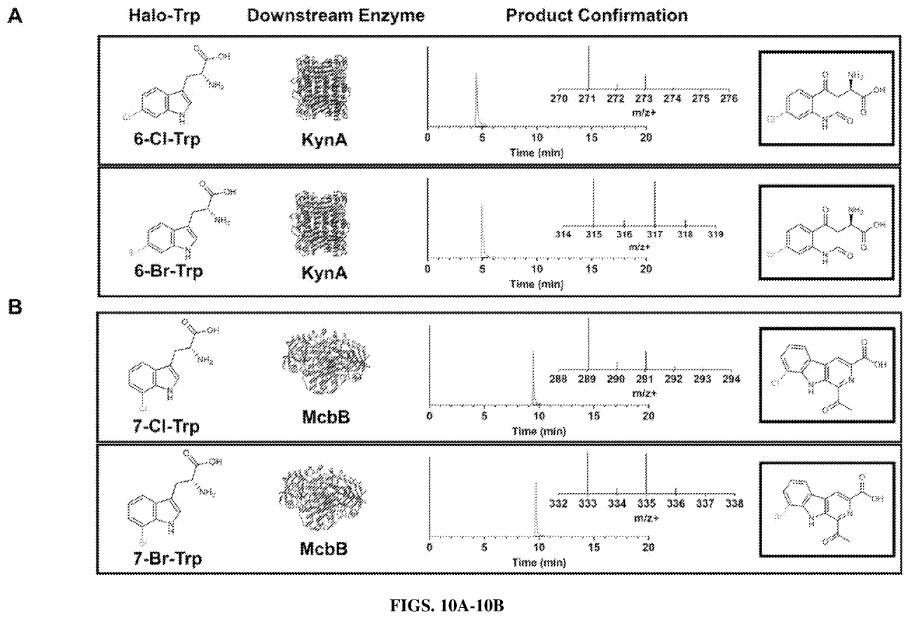

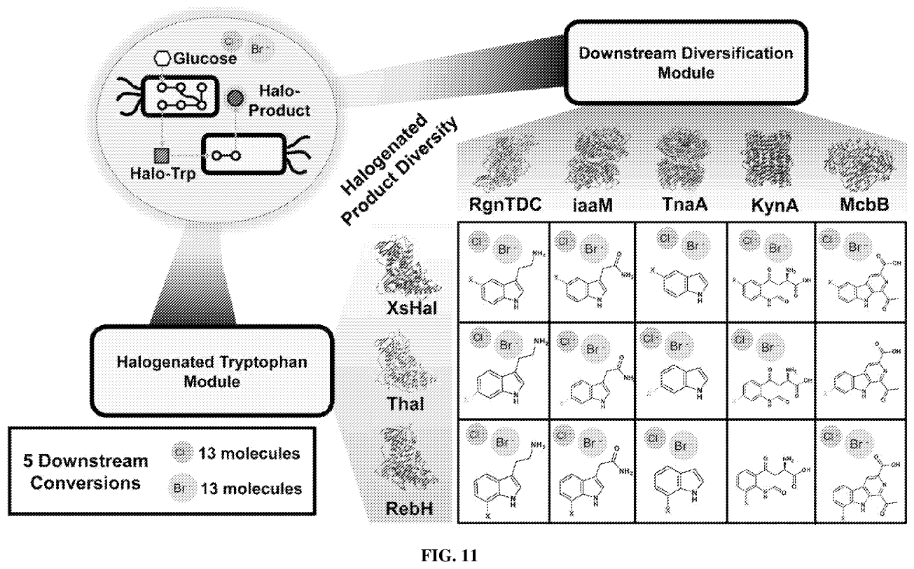

The remaining three enzymes were slightly less promiscuous, each making only 4 out of 6 possible halogenated downstream molecules. KynA was observed to readily convert 6-chloro (1d) and 6-bromo-tryptophan (1e) into 6-chloro-N-formyl-L-kyurenine (5d) and 6-bromo-N-formyl-L-kyurenine (5e), respectively, both precursors to prodrug 4-chloro- and 4-bromo-kynurenine [44][86](FIG. 10A). KynA also exhibits slight promiscuity for the 5-Cl and 5-Br positions (molecules 5b and 5c) but molecules 5f and 5g were not observed. McbB has a much more complicated reaction mechanism and restrictive active site bundled between two subunits [87], yet was able to convert both the 5 and 7-position halo-tryptophan variants, molecules 1a, 1b, 1f, and 1g, to their corresponding carboline products, molecules 6b, 6c, 6f, and 6g, respectively with the 7-position produced at higher efficiency. Molecules 6d and 6e were not observed. Previous report have observed McbB's ability to convert certain fluorine-modified tryptophans [76], though this represents confirmation of larger halogen substituted tryptophans. The four observed molecules are new-to-nature chloro- and bromo-modified beta-carbolines, with the 7-position highlighted in FIG. 10B. Lastly, TsrM follows a similar promiscuity pattern as McbB, where molecules 7b, 7c, 7f, and 7g were observed, corresponding to promiscuity for the 5 and 7-positions, whereas molecules 7d and 7e were not observed, reflecting similar trends seen in vitro [83]. The potential for di-halogenation of these downstream molecules was also investigated and was not evident based on the resulting LC-MS data. While the study was not able to obtain analytical standards for the majority of downstream halogenated molecules, the study provided estimated titers and conversions of fed substrates from the feeding assay based on consumption of fed substrate (TABLE 6 and TABLE 8).

These feeding assays coupled with downstream reaction promiscuity demonstrates the chemical diversity possible from tryptophan, whereby the tryptophan scaffold can be chemically decorated in a variety of ways. In general, it was observed that promiscuity was related to the distance of the halogen from the specific reaction center in addition to the simplicity of the reaction mechanism. For example, RgnTDC and iaaM are remarkably promiscuous and both catalyze reactions near the α-carbon. TnaA is also highly promiscuous and catalyzes a simple cleavage reaction of the β-carbon bond from the indole side group. KynA, TsrM, and McbB on the other hand catalyze more complex reactions such as a complex ring opening, a methylation of the C-2 carbon, and ring closing reactions, respectively, within or very close to the indole side group. To further confirm these empirical observations, the study pursued a computational investigation to determine relative binding energies between iaaM and McbB for tryptophan and halo-tryptophan precursors. General promiscuity trends were supported wherein iaaM has a larger binding pocket that can accommodate all variations of tryptophan and McbB's preference for 7-halo-tryptophan over the other positions is highly evident from the conformational change of a tyrosine group in the active site that especially occludes the 6-halo-position (TABLE 9).

TABLE 9
Docking study binding energy scores. Binding energy scores were calculated
for both iaaM and McbB complexed with tryptophan (none) and each halo-tryptophan
variant that was experimentally tested in this study. Each enzyme's ability
to bind the halo-trps (ΔΔG of binding) was compared to the enzyme's
ability to bind non-halogenated tryptophan (ΔΔG of binding).

| Receptor | Position | Halogen | Binding energy (REU) | Normalized binding energy |
|---|---|---|---|---|
| iaaM | None | None | −139.0819818 | 1 |
| | 5 | Br | −31.63990489 | 0.229439306 |
| | | Cl | −30.05327287 | 0.22083995 |
| | 6 | Br | −35.04231958 | 0.257561137 |
| | | Cl | −32.31333541 | 0.233504574 |
| | 7 | Br | −29.42593187 | 0.219063975 |
| | | Cl | −28.22415826 | 0.214507904 |
| McbB | None | None | −234.2799555 | 1 |
| | 5 | Br | −26.70300691 | 0.113979051 |
| | | C | −27.50496442 | 0.117402124 |
| | 6 | Br | −30.52214523 | 0.130280651 |
| | | Cl | −29.31562485 | 0.125130743 |
| | 7 | Br | −30.30724716 | 0.129363381 |
| | | Cl | −29.9880519 | 0.128000929 |

Computational investigation confirms enzyme promiscuity trends observed experimentally: Enzymes iaaM and McbB complexed with tryptophan gave binding energies of −139 and −235 REU (Rosetta Energy Units) respectively with the given score function. To determine the relative ability of each enzyme to accommodate the halogenation on tryptophan, the study compared the enzyme's ability to bind the halo-trps (AAG of binding) to the enzyme's ability to bind normal tryptophan (AAG of binding). These two terms were divided, creating a normalized binding potential. This results in a score where 1 represents a binding potential that is near native, 0 represents no binding, and a negative score represents a binding that has a positive AAG. The results are shown in TABLE 9.

Computationally, iaaM has a greater ability to accommodate halogenated tryptophan substrates in relation to its native substrate when compared to McbB. Additionally, the non-normalized magnitude of the binding energy for iaaM is also greater than McbB, further supporting this claim. For both enzymes, the binding of different types of halogenated tryptophan, varying in both position and halogen group, were comparable. From these results, the study concluded that, computationally, iaaM is more promiscuous than McbB, and thus able to better accommodate the halogen group on tryptophan. This aligns with the experimental results of this study, where McbB was unable to convert any of the 6-chloro- or 6-bromo-tryptophan whereas iaaM could convert all halogenated substrates with very high relative conversion based on HPLC peak heights and LCMS intensities. Interestingly, a significant drop in binding potential for McbB on the 6-halo-position substrates was not observed, which did not turnover experimentally. However, by inspecting the Rosetta docking structures, one can draw a few conclusions about the effect of halogenation on the binding of the tryptophan substrates. For the McbB structures, for both a 5-halo- and 6-halosubstitution, there is a major confirmational change in the Tyr216 due to the introduction of a nearby halogen group, with this residue closest to the 6-position. This is a potential explanation for the experimental data, where the 5-halo- and, especially, 6-halo-substituted substrates show a lesser promiscuity than the 7-halo-substituted substrates. IaaM appears to have a much less compact binding site near the 5, 6, and 7-halo-positions of tryptophan, with no major confirmational changes occurring in the side chains of IaaM with differently substituted tryptophans. This is a possible explanation for the generally increased promiscuity of IaaM when compared to McbB. Differences in binding energy between the various halogenated tryptophan alongs were not observed in the same enzyme. Allowance for major shifts in the position of the substrate or the confirmation of the active site could be attempted in future studies to further elucidate nuanced binding trends. However, certain halogenated positions may have significant effects on the enzyme's reaction mechanism and could thus not be determined through binding studies alone. In all, high level promiscuity trends were corroborated through computational investigation and similar studies could act to narrow down large sets of enzymes to rapidly predict the most promiscuous variants.

De novo production of halogenated products using synthetic, modular co-cultures: To enable true de novo production of rapidly diversified halogenated molecules, the study sought to utilize a co-culture approach (FIG. 11), which represents a break from the convention of most previous downstream diversification of halogenated tryptophan works focused on combined pathway engineering within single cells [46][47][71]. Given that the halogenated tryptophan molecule is secreted from the cell, this point represents a natural break in metabolic pathways. Thus, combining a halogenated tryptophan overproduction strain with a downstream conversion strain can enable de novo production of an array of halogenated compounds. Moreover, it was hypothesized that this co-culture approach was necessary as a consolidated bioprocessing approach (wherein halogenase and downstream enzyme are co-localized) would lead to high competition for the intracellular tryptophan pool by both competing pathways and result in more un-halogenated products. Similar approaches have been used to reduce metabolic burden and generate a variety of products downstream of tryptophan, such as tryptamine and indigo [88][89]. Thus, one cell can act to primarily produce halogenated tryptophan whereas the other cell can specialize on the downstream conversion of halogenated tryptophan.

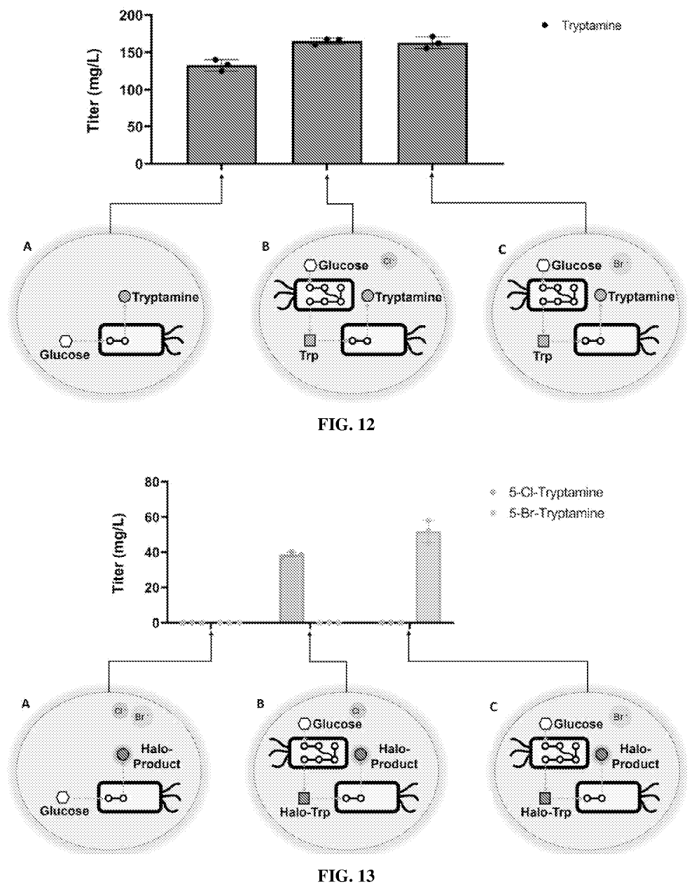

To determine the effectiveness of the modular co-culture reactions, the study compared the production of the non-halogenated down-stream molecule using the wild-type tryptophan pool to that of the non-halogenated product formed during the co-culture reactions with both the halo-trp overproduction strain and downstream conversion strain. The study evaluated this strategy for molecules where standards were available (5-chloro- and 5-bromo-tryptamine). As compared to a blank plasmid control, it was discovered that all the halo-tryptophan was readily converted into the equivalent halo-tryptamine molecule with a minimal increase in amount of the non-halogenated tryptamine product (FIG. 12 and FIG. 13). Thus, the spatial separation effectively enables the generation of primarily the halogenated version. At the same time, the study confirmed de novo production of 36 mg/L of 5-chloro-tryptamine and 52 mg/L of 5-bromo-tryptamine at the 1 mL scale.

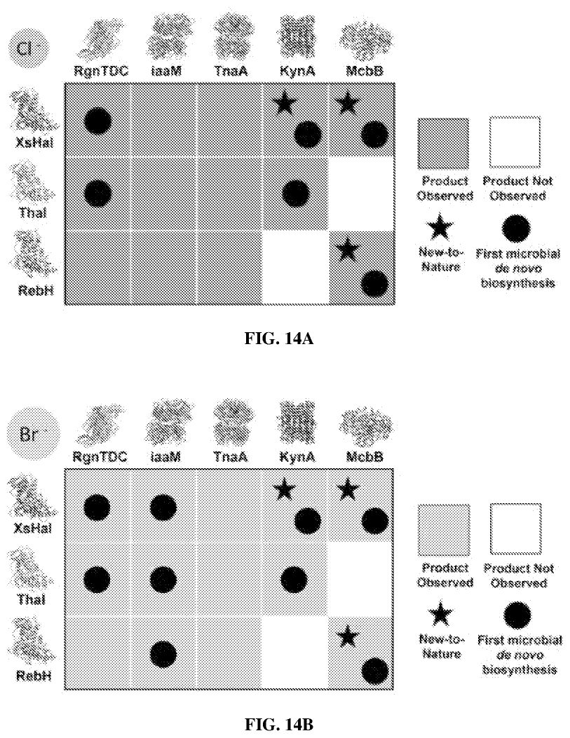

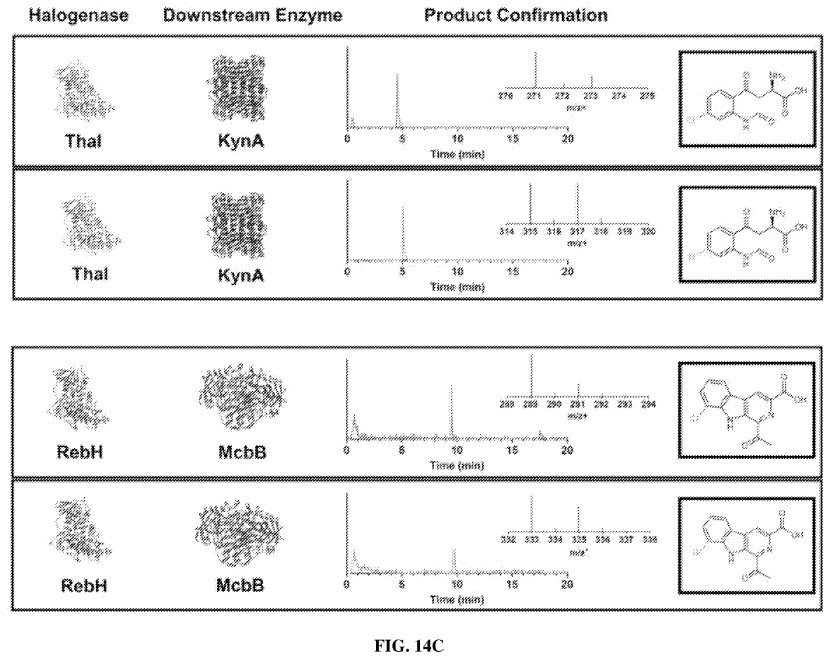

By deploying these modular, one-pot de novo co-culture reactions, the study has confirmed the production of 26 distinct halogenated molecules from a glucose feedstock (FIGS. 14A-14C). As expected, the de novo production results echo the feeding assays, where certain positions and enzymes are more promiscuous than others. TsrM was excluded from these experiments due to the low conversion of the native tryptophan substrate during the feeding assays. These de novo production schemes unlocked new access to synthesis from glucose in a microbial host of many of these products at the time the study was completed, highlighted with a circle (FIGS. 14A-14C). Among these products include new-to-nature molecules, highlighted with a star, such as precursors to kynurenine prodrugs and the 5-chloro-, 5-bromo-, 7-chloro-, and 7-bromo-1-acetytl-3-carboxy-β-carboline molecules that provide pathways to halogenated molecules that can serve as anti-inflammatory agents. While the study not able to obtain analytical standards for the majority of these products to enable full quantification, the study provided estimated titers of all products successfully produced via this co-culture format based on relative LCMS abundance areas and feeding assay estimated titers (TABLE 6). These estimated titers have been benchmarked against exact titers for 5-Cl-Tryptamine and 5-Br-Tryptamine, quantified via use of commercially available analytical standards (TABLE 7). The estimated 5-Br-Tryptamine titer falls within 5% of the exact titer, while the estimated 5-Cl-Tryptamine titer exceeds the exact titer by ˜50%. This indicates that the estimation accuracy likely varies with each compound. However, these estimates provide a general sense of titer scale (e.g., 1 mg/L vs 10 mg/L vs 100 mg/L), providing a best approximation in the absence of available reference standards for the majority of downstream halogenated compounds produced in this study. To provide clarity on the purity of compounds produced via co-culture, the study also provided estimated yields based on produced downstream molecule divided by total product, tryptophan and/or halo-tryptophan formed in the co-culture reaction (equal to product formed+residual tryptophan/halo-tryptophan; TABLE 10).

TABLE 10
Estimated yield of downstream products from cocultures. Estimated
yields were calculated by dividing the estimated downstream
product titers by the sum of the produced downstream product,
and residual tryptophan and/or halogenated tryptophan precursors.
The sum of the produced downstream product, and residual tryptophan
and/or halogenated tryptophan precursors is taken to represent
the maximum mM of product possible for each respective coculture,
with a maximum achievable yield of 100%. Coculture estimated
yield was estimated by dividing estimated product titer from
TABLE 7 by the sum of final product titer [mM] +
residual halo-tryptophan [mM] + residual tryptophan [mM].

| Product | Coculture Estimated Yield[%] |
|---|---|
| Tryptamine | 100 |
| 5-Cl-Tryptamine | 100 |
| 5-Br-Tryptamine | 98.2 |
| 6-Cl-Tryptamine | 100 |
| 6-Br-Tryptamine | 100 |
| 7-Cl-Tryptamine | 100 |
| 7-Br-Tryptamine | 100 |
| Indole | 92.9 |
| 5-Cl-Indole | 100 |
| 5-Br-Indole | 99.6 |
| 6-Cl-Indole | 100 |
| 6-Br-Indole | 100 |
| 7-Cl-Indole | 55.1 |
| 7-Br-Indole | 71 |
| Indole-3-Acetamide | 100 |
| 5-Cl-Indole-3-Acetamide | 100 |
| 5-Br-Indole-3-Acetamide | 99.2 |
| 6-Cl-Indole-3-Acetamide | 100 |
| 6-Br-Indole-3-Acetamide | 100 |
| 7-Cl-Indole-3-Acetamide | 100 |
| 7-Br-Indole-3-Acetamide | 100 |
| N-Formyl-L-Kynurenine | 72.7 |
| 5-Cl-N-Formy1-L-Kynurenine | 20.8 |
| 5-Br-N-Formyl-L-Kynurenine | 11.1 |
| 6-Cl-N-Formyl-L-Kynurenine | 29.4 |
| 6-Br-N-Formyl-L-Kynurenine | 14.2 |
| 7-Cl-N-Formyl-L-Kynurenine | 0 |
| 7-Br-N-Formyl-L-Kynurenine | 0 |
| 1-acetyl-3-carboxy-B-carboline | 20.8 |
| 5-Cl-1-acetyl-3-carboxy-B-carboline | 10.7 |
| 5-Br-1-acetyl-3-carboxy-B-carboline | 6.5 |
| 6-Cl-1-acetyl-3-carboxy-B-carboline | 0 |
| 6-Br-1-acetyl-3-carboxy-B-carboline | 0 |
| 7-Cl-1-acetyl-3-carboxy-B-carboline | 1.2 |
| 7-Br-1-acetyl-3-carboxy-B-carboline | 0.7 |
| 2-methyl-L-tryptophan | 0 |
| 5-Cl-2-methyl-L-tryptophan | 0 |
| 5-Br-2-methyl-L-tryptophan | 0 |
| 6-Cl-2-methyl-L-tryptophan | 0 |
| 6-Br-2-methyl-L-tryptophan | 0 |
| 7-Cl-2-methyl-L-tryptophan | 0 |
| 7-Br-2-methyl-L-tryptophan | 0 |

### DISCUSSION

Bioproduction offers a green solution for the generation of a diverse range of functional products. Disclosed herein are engineered E. coli as a microbial platform to convert glucose into a wide diversity of halogenated tryptophan derivatives. It was showcased that through various engineering approaches, platform strains capable of producing six different halogenated tryptophan precursors de novo from glucose at high milligram-per-liter scales in flasks can rapidly be developed. This platform opens the door to many more applications and pushes the envelope towards reaching commercial viability for de novo biosynthesis of diverse halogenated molecules. By then investigating native promiscuity of six disparate downstream reactions in vivo, the study showed that many enzymes are amenable to further convert halogenated tryptophan precursors into a variety of halogenated downstream products in vivo, largely consistent with previous reports of promiscuity [48][50][53][70][71]. Lastly, the study showed that the halogenated tryptophan overproduction strains and strains equipped with downstream enzymes can be combined in a modular co-culture fashion to generate over 26 distinct halogenated molecules including 15 first-time de novo biosynthesized, of which 6 are entirely new-to-nature products. Future investigation of promiscuity for the pathway enzymes is warranted, where quantification via in vitro reactions could enable a better understanding of each enzyme's ability to turnover halogenated tryptophan variants. Additionally, pursuits to engineer and evolve native selectivity in favor of the halogenated variant of interest could prove fruitful, similar to the enzymes that have already evolved in nature, such as Tar13, that are more specific for halogenated tryptophan than for tryptophan [44]. Taken together, this platform demonstrates a synthetic bio-combinatorial chemistry approach to yield de novo production of halogenated compounds of relevance to a variety of industrial sectors.

### EXAMPLE ASPECTS

Example 1: A consortium of engineered microorganisms, comprising: at least one upstream engineered microorganism for producing halogenated tryptophan; and at least one downstream engineered microorganism for converting said halogenated tryptophan into a halogenated tryptophan-derived product.

Example 2: The consortium of any examples herein, particularly Example 1, wherein the at least one upstream engineered microorganism converts a carbon source to tryptophan and subsequently converts said tryptophan to halogenated tryptophan.

Example 3: The consortium of any examples herein, particularly Example 1, wherein a first upstream engineered microorganism converts a carbon source to tryptophan; and wherein a second upstream engineered microorganism subsequently converts said tryptophan to halogenated tryptophan.

Example 4: The consortium of any examples herein, particularly Examples 1-3, wherein the at least one upstream engineered microorganism expresses at least one halogenase.

Example 5: The consortium of any examples herein, particularly Example 4, wherein the at least one halogenase is PyrH, XsHal, Thal, Th-Hal, SttH, RebH, PrnA, AetF, or any combination thereof.

Example 6: The consortium of any examples herein, particularly Examples 4-5, wherein the at least one upstream engineered microorganism further expresses an enzyme for generating a cofactor for the at least one halogenase.

Example 7: The consortium of any examples herein, particularly Example 6, wherein the cofactor is flavin adenine dinucleotide (FADH₂), and wherein the enzyme for generating the cofactor is a flavin reductase.

Example 8: The consortium of any examples herein, particularly Example 7, wherein the flavin reductase is an E. coli flavin reductase.

Example 9: The consortium of any examples herein, particularly Examples 1-8, wherein the halogenated tryptophan is chlorinated or brominated.

Example 10: The consortium of any examples herein, particularly Example 9, wherein the halogenated tryptophan comprises 5-chloro-tryptophan, 6-chloro-tryptophan, 7-chloro-tryptophan, 5,7-dichloro-tryptophan, 5-bromo-tryptophan, 6-bromo-tryptophan, 7-bromo-tryptophan, 5,7-dibromo-tryptophan, or any combination thereof.

Example 11: The consortium of any examples herein, particularly Examples 1-10, wherein the at least one downstream engineered microorganism expresses at least one downstream enzyme.

Example 12: The consortium of any examples herein, particularly Example 11, wherein the at least one downstream enzyme is promiscuous.

Example 14: The consortium of any examples herein, particularly Examples 11-13, wherein a first downstream enzyme converts the halogenated tryptophan into a halogenated intermediate; and wherein a second downstream enzyme converts the halogenated intermediate into the halogenated tryptophan-derived product.

Example 15: The consortium of any examples herein, particularly Example 14, wherein the first downstream enzyme is RgnT, RgnTD, or any combination thereof; and wherein the second downstream enzyme is RgnDC, RgnC, or any combination thereof.

Example 16: The consortium of any examples herein, particularly Examples 11-13, wherein the at least one downstream enzyme directly converts the halogenated tryptophan to the halogenated tryptophan-derived product.

Example 17: The consortium of any examples herein, particularly Example 16, wherein the at least one downstream enzyme is iaaM, TnaA, KynA, McbB, or any combination thereof.

Example 18: The consortium of any examples herein, particularly Examples 1-17, wherein the halogenated tryptophan-derived product comprises halo-tryptamine, halo-indole-3-acetamide, halo-indole, halo-N-formyl-L-kynurenine, halo-1-acetyl-3-carboxy-β-carboline, halo-2-methyl-L-tryptophan, or any combination thereof.

Example 19: The consortium of any examples herein, particularly Examples 1-18, wherein each of the at least one upstream engineered microorganism and the at least one downstream engineered microorganism is a bacterium.

Example 20: The consortium of any examples herein, particularly Example 19, wherein the bacterium is E. coli or C. glutamicum.

Example 21: The consortium of any examples herein, particularly Examples 1-20, wherein the at least one upstream engineered microorganism and the at least one downstream engineered microorganism are separately cultured.

Example 22: A method of making a tryptophan-derived product, the method comprising: a) providing the consortium of any examples herein, particularly Examples 1-21; b) exposing the at least one upstream engineered microorganism to a feedstock, thereby producing a halogenated tryptophan; and c) exposing the at least one downstream engineered microorganism to the halogenated tryptophan, thereby converting the halogenated tryptophan to a halogenated tryptophan-derived product.

Example 23: The method of any examples herein, particularly Example 22, wherein the at least one upstream engineered microorganism and the at least one downstream engineered microorganism are separately cultured; and wherein step b) further comprises collecting the halogenated tryptophan produced by the at least one upstream engineered microorganism.

Example 24: The method of any examples herein, particularly Examples 22-23, wherein the feedstock comprises a carbon source, and wherein the at least one upstream engineered microorganism converts the carbon source to tryptophan.

Example 25: The method of any examples herein, particularly Examples 22-24, wherein the feedstock comprises tryptophan.

Example 26: The method of any examples herein, particularly Examples 22-25, wherein the at least one upstream engineered microorganism produces about 100 mg/L or greater of the halogenated tryptophan.

Example 27: The method of any examples herein, particularly Examples 22-26, wherein the at least one downstream engineered microorganism produces about 100 mg/L or greater of the halogenated tryptophan-derived product.

Example 28: A halogenated tryptophan-derived product generated by the method of any examples herein, particularly Examples 22-27.

Example 29: The halogenated tryptophan-derived product of any examples herein, particularly Example 28, wherein the halogenated tryptophan-derived product comprises halo-tryptamine, halo-indole-3-acetamide, halo-indole, halo-N-formyl-L-kynurenine, halo-1-acetyl-3-carboxy-β-carboline, halo-2-methyl-L-tryptophan, or any combination thereof.

The following patents, applications and publications as listed below and throughout this document are hereby incorporated by reference in their entirety herein.

### REFERENCE LIST

1. Reed, K. B. et al. Expanding beyond canonical metabolism: interfacing alternative elements, synthetic biology, and metabolic engineering. Synth. Syst. Biotechnol. 3, 20-33 (2018).
2. Chellan, P. et al. The elements of life and medicines. Philos. Trans. A Math. Phys. Eng. Sci. 373, 20140182 (2015).
3. Wackett, L. P. et al. Microbial genomics and the periodic table. Appl. Environ. Microbiol. 70, 647-655 (2004).
4. Goss, R. J. et al. The generation of “unNatural” products: synthetic biology meets synthetic chemistry. Nat. Prod. Rep. 29, 870-889 (2012).
5. Jackie Tsoi, C. et al. Combinatorial biosynthesis of ‘unnatural’ natural products: the polyketide example. Chem. Biol. 2, 355-362 (1995).
6. Walker, M. C. et al. Natural and engineered biosynthesis of fluorinated natural products. Chem. Soc. Rev. 43, 6527-6536 (2014).
7. Latham, J. et al. Development of halogenase enzymes for use in synthesis. Chem. Rev. (2017).
8. van Pee, K.-H. Biosynthesis of halogenated metabolites by bacteria. Annu. Rev. Microbiol. 50, 375-399 (1996).
9. Bradley, S. A. et al. Deploying microbial synthesis for halogenating and diversifying medicinal alkaloid scaffolds. Front. Bioeng. Biotechnol. 8, 594126 (2020).
10. Gkotsi, D. S. et al. A marine viral halogenase that iodinates diverse substrates. Nat. Chem. (2019).
11. Ayala, M. et al. Halogenases: a biotechnological alternative for the synthesis of halogenated pharmaceuticals. Mini-Rev. Med. Chem. 16, 1100-1111 (2016).
12. Heidrich, J. et al. Embracing the diversity of halogen bonding motifs in fragment-based drug discovery—construction of a diversity-optimized halogen-enriched fragment library. Front. Chem. 7, 9 (2019).
13. Benedetto Tiz, D. et al. New halogen-containing drugs approved by FDA in 2021: an overview on their syntheses and pharmaceutical use. Molecules 27, 1643 (2022).
14. Hernandes, M. Z. et al. Halogen atoms in the modern medicinal chemistry: hints for the drug design. Curr. Drug Targets 11, 303-314 (2010).
15. Jeschke, P. Manufacturing approaches of new halogenated agro-chemicals. Eur. J. Org. Chem. 2022, e202101513 (2022).
16. Jeschke, P. The unique role of halogen substituents in the design of modern agrochemicals. Pest Manag. Sci. 66, 10-27 (2010).
17. B0chler, J. et al. Recent advances in flavin-dependent halogenase biocatalysis: sourcing, engineering, and application. Catalysts 9, 1030 (2019).
18. Saccone, M. et al. Halogen bonding beyond crystals in materials science. J. Phys. Chem. B 123, 9281-9290 (2019).
19. Kampes, R. et al. Halogen bonding in polymer science: towards new smart materials. Chem. Sci. 12, 9275-9286 (2021).
20. Ye, W. et al. Halogen-based functionalized chemistry engineering for high-performance supercapacitors. Chin. Chem. Lett. (2022)
21. Biswas, S. et al. Recent developments in polymeric assemblies and functional materials by halogen bonding. ChemNanoMat 7, 748-772 (2021).
22. Cavallo, G. et al. The halogen bond. Chem. Rev. 116, 2478-2601 (2016).
23. Podgorsek, A. et al. Oxidative halogenation with “green” oxidants: oxygen and hydrogen peroxide. Angew. Chem. Int. Ed. 48, 8424-8450 (2009).
24. Neumann, C. S. et al. Halogenation strategies in natural product biosynthesis. Chem. Biol. 15, 99-109 (2008).
25. Zaragoza Dörwald, F. et al. Electrophilic halogenation of arenes. in Side Reactions in Organic Synthesis II 121-160 (John Wiley & Sons, Ltd, 2014).
26. Brown, S. et al. Halogenase engineering for the generation of new natural product analogues. ChemBioChem 16, 2129-2135 (2015).
27. Agarwal, V. et al. Enzymatic halogenation and dehalogenation reactions: pervasive and mechanistically diverse. Chem. Rev. 117, 5619-5674 (2017).
28. Vaillancourt, F. H. et al. SyrB2 in syringomycin E biosynthesis is a nonheme Fell α-ketoglutarate- and O2-dependent halogenase. Proc. Natl. Acad. Sci. USA 102, 10111-10116 (2005).
29. Neugebauer, M. E. et al. A family of radical halogenases for the engineering of amino-acid-based products. Nat. Chem. Biol. 15, 1009-1016 (2019).
30. Zhu, Q. et al. Aliphatic halogenase enables late-stage C—H functionalization: selective synthesis of a brominated fischerindole alkaloid with enhanced antibacterial activity. Chembiochem 17, 466-470 (2016).
31. Menon, B. R. K. et al. RadH: a versatile halogenase for integration into synthetic pathways. Angew. Chem. Int. Ed. (2017).
32. Zeng, J. et al. A novel fungal flavin-dependent halogenase for natural product biosynthesis. ChemBioChem 11, 2119-2123 (2010).
33. Wang, S. et al. Metabolic engineering of Escherichia coli for the biosynthesis of various phenylpropanoid derivatives. Metab. Eng. 29, 153-159 (2015).
34. Fraley, A. E. et al. Function and structure of MalA/MalA′, iterative halogenases for late-stage C—H functionalization of indole alkaloids. J. Am. Chem. Soc. 139, 12060-12068 (2017).
35. Zehner, S. et al. A regioselective tryptophan 5-halogenase is involved in pyrroindomycin biosynthesis in Streptomyces rugosporus LL-42D005. Chem. Biol. 12, 445-452 (2005).
36. Seibold, C. et al. A flavin-dependent tryptophan 6-halogenase and its use in modification of pyrrolnitrin biosynthesis. Biocatal. Bio-transformation 24, 401-408 (2006).
37. Yeh, E. et al. Robust in vitro activity of RebF and RebH, a two-component reductase/halogenase, generating 7-chlorotryptophan during rebeccamycin biosynthesis. Proc. Natl. Acad. Sci. USA 102, 3960-3965 (2005).
38. Frese, M. et al. Enzymatic halogenation of tryptophan on a gram scale. Angew. Chem. Int. Ed. 54, 298-301 (2015).
39. Veldmann, K. H. et al. Bromination of L-tryptophan in a fermentative process with Corynebacterium glutamicum. Front. Bioeng. Biotechnol. 7, 219 (2019).
40. Almeida, M. C. et al. Tryptophan derived natural marine alkaloids and synthetic derivatives as promising antimicrobial agents. Eur. J. Med. Chem. 209, 112945 (2021).
41. Roszkowski, P. et al. Variety of natural products derived from tryptophan and stereoselective synthesis of tetrahydro-β-carboline derivatives of pharmacological importance. Int. Congr. Ser. 1304, 46-59 (2007).
42. Alkhalaf, L. M. et al. Biosynthetic manipulation of tryptophan in bacteria: pathways and mechanisms. Chem. Biol. 22, 317-328 (2015).
43. Lenz, C. et al. Taking different roads: 1-tryptophan as the origin of psilocybe natural products. ChemPlusChem 86, 28-35 (2021).
44. Luhavaya, H. et al. Biosynthesis of 1-4-chlorokynurenine, an antidepressant prodrug and a non-proteinogenic amino acid found in lipopeptide antibiotics. Angew. Chem. Int. Ed. 58, 8394-8399 (2019).
45. Sinchez, C. et al. Combinatorial biosynthesis of antitumor indolo-carbazole compounds. Proc. Natl. Acad. Sci. USA 102, 461-466 (2005).
46. Sharma, S. V. et al. Living GenoChemetics by hyphenating synthetic biology and synthetic chemistry in vivo. Nat. Commun. 8, 229 (2017).
47. Runguphan, W. et al. Integrating carbon-halogen bond formation into medicinal plant metabolism. Nature 468, 461-464 (2010).
48. Frabel, S. et al. Engineering of new-to-nature halogenated indigo precursors in plants. Metab. Eng. 46, 20-27 (2018).
49. Davis, K. et al. Nicotiana benthamiana as a transient expression host to produce auxin analogs. Front. Plant Sci. 11, 581675 (2020).
50. Torrens-Spence, M. P. et al. Engineering new branches of the kynurenine pathway to produce oxo-(2-amino-phenyl) and quinoline scaffolds in yeast. ACS Synth. Biol. 8, 2735-2745 (2019).
51. Li, Y. et al. Complete biosynthesis of noscapine and halogenated alkaloids in yeast. Proc. Natl Acad. Sci. USA. (2018).
52. Veldmann, K. H. et al. Metabolic engineering of Corynebacterium glutamicum for the fermentative production of halogenated tryptophan. J. Biotechnol. 291, 7-16 (2019).
53. Lee, J. et al. Production of Tyrian purple indigoid dye from tryptophan in Escherichia coli. Nat. Chem. Biol. (2020).
54. Dong, C. et al. Tryptophan 7-halogenase (PrnA) structure suggests a mechanism for regioselective chlorination. Science 309, 2216-2219 (2005).
55. Menon, B. R. K. et al. Structure and biocatalytic scope of thermophilic flavin-dependent halogenase and flavin reductase enzymes. Org. Biomol. Chem. 14, 9354-9361 (2016).
56. Domergue, J. et al. XszenFHal, a novel tryptophan 5-halogenase from Xenorhabdus szentirmaii. AMB Expr. 9, 175 (2019).
57. Rodriguez, A. et al. Engineering Escherichia coli to overproduce aromatic amino acids and derived compounds. Microb. Cell Fact. 13, 126 (2014).
58. Gunsalus, R. P. et al. Nucleotide sequence and expression of Escherichia coli trpR, the structural gene for the trp aporepressor. (1980).
59. Gu, P. et al. The improved 1-tryptophan production in recombinant Escherichia coli by expressing the polyhydroxybutyrate synthesis pathway. Appl. Microbiol. Biotechnol. 97, 4121-4127 (2013).
60. Dodge, T. C. et al. Optimization of the glucose feed rate profile for the production of tryptophan from recombinant E. coli. J. Chem. Technol. Biotechnol. 77, 1238-1245 (2002).
61. Chen, L. et al. Rational design and metabolic analysis of Escherichia coli for effective production of L-tryptophan at high concentration. Appl. Microbiol. Biotechnol. 101, 559-568 (2017).
62. Liu, L. et al. Metabolic engineering and fermentation process strategies for L-tryptophan production by Escherichia coli. Processes 7, 213(2019).
63. Reed, K. B. et al. Modular biocatalysis for polyamines. Nat. Catal. 4,449-450 (2021).
64. Tyzack, J. D. et al. Exploring chemical biosynthetic design space with transform-MinER. ACS Synth. Biol. 8, 2494-2506 (2019).
65. Schultz, A. W. et al. Functional characterization of the cyclomarin/cyclomarazine prenyltransferase CymD directs the biosynthesis of unnatural cyclic peptides. J. Nat. Prod. 73, 373-377 (2010).
66. Nielsen, C. A. et al. The important ergot alkaloid intermediate chanoclavine-I produced in the yeast Saccharomyces cerevisiae by the combined action of EasC and EasE from Aspergillus japonicus. Microb. Cell Fact. 13, 95 (2014).
67. Kremer, A. et al. A 7-dimethylallyltryptophan synthase from Aspergillus fumigatus: overproduction, purification and biochemical characterization. Microbiology 153, 3409-3416 (2007).
68. Tittarelli, R. et al. Recreational use, analysis and toxicity of tryptamines. Curr. Neuropharmacol. 13, 26-46 (2015).
69. Ehrenworth, A. M. et al. Accelerating the semisynthesis of alkaloid-based drugs through metabolic engineering. Nat. Chem. Biol. 13, 249-258 (2017).
70. McDonald, A. D. et al. Facile in vitro biocatalytic production of diverse tryptamines. Chembiochem 20, 1939-1944 (2019).
71. Menon, N. et al. Versatile and facile one-pot biosynthesis for amides and carboxylic acids in E. coli by engineering auxin pathways of plant microbiomes. ACS Catal. 12,2309-2319 (2022).
72. Sanna, G. et al. Synthesis and biological evaluation of novel indole-derived thioureas. Molecules 23, 2554 (2018).
73. Ullrich, R. et al. Synthesis of indigo-dyes from indole derivatives by unspecific peroxygenases and their application for in-situ dyeing. Catalysts 11, 1495 (2021).
74. El-Najjar, N. et al. The chemical and biological activities of quinones: overview and implications in analytical detection. Phytochem. Rev. 10, 353 (2011).
75. Muneer, A. Kynurenine pathway of tryptophan metabolism in neuropsychiatric disorders: pathophysiologic and therapeutic considerations. Clin. Psychopharmacol. Neurosci. 18, 507-526 (2020).
76. Chen, Q. et al. Discovery of McbB, an enzyme catalyzing the β-carboline skeleton construction in the marinacarboline biosynthetic pathway. Angew. Chem. Int. Ed. Engl. 52, 9980-9984 (2013).
77. Aaghaz, S. et al. O-Carbolines as potential anticancer agents. Eur. J. Med. Chem. 216, 113321 (2021).
78. Szab6, T. et al. Recent advances in the synthesis of β-carboline alkaloids. Molecules 26, 663 (2021).
79. Piechowska, P. et al. Bioactive β-carbolines in food: a review. Nutrients 11, 814 (2019).
80. Singh, M. et al. Catalyst-free and metal-free approach towards synthesis of amide- and thioamide-linked β-carboline-pyridine conjugates and estimation of their photo-physical properties. ChemistrySelect 5, 5172-5179 (2020).
81. Huang, H. et al. Antimalarial β-carboline and indolactam alkaloids from Marinactinospora thermotolerans, a deep sea isolate. J. Nat. Prod. 74, 2122-2127 (2011).
82. Hou, Y. et al. Pharmacodynamics assessment of β-carboline from the roots of Psammosilene tunicoides as analgesic compound. J. Ethnopharmacol. 291, 115163 (2022).
83. Benjdia, A. et al. The thiostrepton A tryptophan methyltransferase TsrM catalyses a cob(II)alamin-dependent methyl transfer reaction. Nat. Commun. 6, 8377(2015).
84. Pierre, S. et al. Thiostrepton tryptophan methyltransferase expands the chemistry of radical SAM enzymes. Nat. Chem. Biol. 8, 957-959 (2012).
85. Knox, H. L. et al. Structural basis for non-radical catalysis by TsrM, a radical SAM methylase. Nat. Chem. Biol. (2021).
86. Wu, Y. et al. Rational design of a de novo enzyme cascade for scalable continuous production of anti-depressant prodrugs. ACS Catal. (2022)
87. Mori, T. et al. Structural basis for β-carboline alkaloid production by the microbial homodimeric enzyme McbB. Chem. Biol. 22, 898-906 (2015).
88. Wang, X. et al. Developing E. coli-E. coli co-cultures to overcome barriers of heterologous tryptamine biosynthesis. Metab. Eng. Commun. 10, e00110 (2019).
89. Chen, T. et al. Development and optimization of a microbial co-culture system for heterologous indigo biosynthesis. Microb. Cell Fact. 20, 154 (2021).
90. Serra-Moreno, R. et al. Use of the lambda Red recombinase system to produce recombinant prophages carrying antibiotic resistance genes. BMC Mol. Biol. 7, 31(2006).
91. Gibson, D. G. et al. Enzymatic assembly of DNA molecules up to several hundred kilobases. Nat. Methods 6, 343-345 (2009).
92. Chaudhury, S. et al. PyRosetta: a script-based interface for implementing molecular modeling algorithms using Rosetta. Bioinformatics 26, 689-691 (2010).

SEQUENCES

| | SEQ |
|---|---|
| Sequence | ID NO |
| ggatcctctccttgtgtga | 1 |
| taactcgagagagaatataaaaagcc | 2 |
| ggataacaatttcacacaaggagaggatccATGCTTAATAATGTCGTTAT | 3 |
| atctggctttttatattctctctcgagttaTTAACGCAGTTGGGTAAA | 4 |
| acaatttcacacaaggagaggatccATGGAACGCCGTAAACGT | 5 |
| atctggctttttatattctctctcgagttaTTACTGAATGCTTGCCAGATATTC | 6 |
| ttgcgccttgagcgacac | 7 |
| agcttgtcgaccctgcattagg | 8 |
| TGTTATTAGTTCGTTACTGGAAGTCCAGTCACCTTGTCAGGAGTATTATCa | 9 |
| ttccggggatccgtcgacc | |
| AAAGCGGGTATAAATTCGCCCATCCGTTGCAGATGGGCGAGTAAGAAGT | 10 |
| Agtgtaggctggagctgcttc | |
| TGTAATATTCACAGGGATCACTGTAATTAAAATAAATGAAGGATTATGT | 11 |
| Aattccggggatccgtcgacc | |
| TGTAGGGTAAGAGAGTGGCTAACATCCTTATAGCCACTCTGTAGTATTA | 12 |
| Agtgtaggctggagctgcttcg | |
| gttcatctttcggttggtgg | 13 |
| gtgtatatctccgaagaccgtaa | 14 |
| gtcgacgagctgttgacaattaatcatcggctcgtataatgtgtggaattgtgagcggataacaatttcacacaaggagag | 15 |
| gatcc | |
| taactcgagagagaatataaaaagccagattattaatccggcttttttattattt | 16 |
| ATGGAACGCCGTAAACGTGAACGTCTGGGCTCTCTGGGTCGCCCGACTA | 17 |
| AGAAAGAACTGCGTATGATTCGTAGCGTGGTAATCGTGGGCGGTGGCAC | |
| GGCTGGCTGGATGACTGCTAGCTATCTGAAAGCGGCTTTCGACGATCGT | |
| ATTGATGTTACTCTGGTTGAATCCGGTAACGTGCGCCGTATTGGTGTTGG | |
| CGAAGCAACCTTCTCTACCGTGCGTCATTTCTTCGATTACCTGGGCCTGG | |
| ACGAACGTGAATGGCTGCCGCGTTGTGCCGGTGGCTACAAACTGGGCAT | |
| CCGTTTTGAAAATTGGTCTGAGCCGGGTGAATACTTCTACCACCCGTTCG | |
| AACGTCTGCGTGTTGTAGACGGCTTCAACATGGCGGAATGGTGGCTGGC | |
| TGTTGGTGACCGCCGCACCAGCTTCTCCGAAGCGTGCTACCTGACTCACC | |
| GTCTGTGCGAAGCTAAACGTGCACCACGTATGCTGGACGGCAGCCTGTT | |
| TGCTTCCCAGGTGGACGAGAGCCTGGGCCGCTCCACTCTGGCGGAGCAG | |
| CGCGCGCAATTCCCGTACGCCTACCACTTCGATGCAGATGAGGTAGCCC | |
| GTTATCTGTCTGAATACGCGATTGCGCGTGGTGTGCGTCACGTGGTCGAT | |
| GATGTGCAGCACGTTGGTCAGGACGAACGCGGTTGGATTAGCGGTGTGC | |
| ACACGAAACAGCACGGCGAGATCTCTGGCGACCTGTTCGTTGACTGCAC | |
| CGGTTTCCGTGGTCTGCTGATCAACCAGACCCTGGGTGGTCGTTTCCAGT | |
| CTTTTTCCGATGTACTGCCGAACAACCGTGCTGTCGCGCTGCGCGTGCCG | |
| CGCGAAAACGACGAAGACATGCGTCCGTATACCACCGCGACCGCTATGA | |
| GCGCTGGTTGGATGTGGACCATCCCGCTGTTCAAACGTGATGGTAACGG | |
| TTACGTATATTCCGACGAATTTATCTCCCCGGAAGAAGCTGAACGTGAA | |
| CTGCGCAGCACTGTAGCGCCTGGCCGTGACGACCTGGAAGCAAACCACA | |
| TCCAAATGCGCATCGGTCGTAACGAACGTACCTGGATCAATAACTGCGT | |
| GGCAGTCGGTCTGTCTGCGGCGTTCGTCGAACCACTGGAAAGCACCGGC | |
| ATCTTCTTCATCCAGCACGCTATCGAACAGCTGGTTAAACACTTCCCGGG | |
| CGAACGTTGGGACCCGGTTCTGATCAGCGCTTATAACGAACGTATGGCC | |
| CACATGGTTGACGGCGTTAAAGAATTTCTGGTTCTGCATTACAAAGGTGC | |
| TCAGCGCGAGGATACCCCGTACTGGAAAGCGGCAAAAACGCGCGCTATG | |
| CCAGACGGCCTGGCTCGCAAACTGGAGCTGTCCGCGAGCCACCTGCTGG | |
| ACGAGCAGACTATCTACCCGTACTACCACGGTTTCGAAACGTATTCTTGG | |
| ATTACCATGAACCTGGGCCTGGGTATTGTCCCAGAACGCCCTCGCCCTGC | |
| CCTGCTGCACATGGATCCAGCTCCGGCACTGGCGGAATTCGAACGTCTG | |
| CGCCGTGAGGGCGACGAACTGATCGCAGCACTGCCGAGCTGCTACGAAT | |
| ATCTGGCAAGCATTCAGTAA | |
| ATGATTAATTCGGTATTGATCGTAGGTGGAGGTACTGCTGGCTGGATGA | 18 |
| CTGCCGCGTATCTGTCGAAAGCGTTTGACAAGAATATCAATATTACCGTC | |
| GTTGAGAGTAAGGAGGTAAAGAAGATTGGGGTGGGCGAAGCCACGTTTT | |
| CTACAGTTCGCCATTACTTTGATTATCTGGGCCTTAACGAATCTGAATGG | |
| CTTCCTGAGTGCTCTGGGAGTTATAAACTTGGTATTCGCTTCGAAAACTG | |
| GGACGGACAAGGAAATCATTTTTACCATCCTTTCGAGCGTTGGGAAGTA | |
| GTAAAGGGCTTCCCGATTTCCGAGTGGTGGCTGTCCAAGAAATTAAAGG | |
| ACCAACGCTTTGATTACGATACCTTTTTGACTCCACACTTGTGCGAGGCC | |
| AAACGCTCGCCGCGCCGCTTGGACGGGAGTTTGTTTGCCCAATCTATTGA | |
| CAAATCGCTTGGCCAAAGCACGCTTGCAGAACAACGCGCTCAGTACCCT | |
| TACGCATATCATTTCGATGCAGACGGTGTTGCGAGTTTCCTTAAACGTTA | |
| TGCCATGAACCGTGGTGTCAAACACATTGAGGACGATGTTACACATGTG | |
| GAGATCGACACTAACGGGAACATCGGTTACTTAGAGGCTAAAATCTATG | |
| GAAAATTGCGCGCCGACTTATACATCGATTGTACGGGGTTTAAGGGTCTT | |
| TTAATCAACAAAGCCTTAAACGAACCATTTATCTCGTTTTCCGACGTATT | |
| AAAGAACAACCGCGCTGTGGCCCTTCGTGTACCGCGTGAGAATGAGAAC | |
| GATATCGAACCCTATACGACAGCCCGTACAATGTCTAATGGATGGCGTT | |
| GGACGATCCCACTGTACAAGCGTAATGGCTACGGTTATGTTTACTGCAAT | |
| AAATATCAGTCTCCGGAGGAAGCAGAAATGGAGTTGCGTAAATCGATCC | |
| CTTATGAAAACGCGGAATGCGTAGCCAACCATATCCGCATGCGTATTGG | |
| GCGTTCAAACCGTAGCTGGGTCAAGAACTGCGTTGCAATTGGGCTTTCAT | |
| CTGCGTTCGTGGAACCACTGGAGAGTACGGGGATCTTTTTTATTCAGCAC | |
| GGGATCGAGCAACTTGTGCGCTTCTTTCCGCGCTCCTCGGGCAACGAATT | |
| GCTTATCGAGGAATATAACACGCGCGTTAATCGCGTAGTCGATGGGGTG | |
| AAGGAATTTCTGTTACTTCACTTCTCGCTGGCACAGCGTAACGATACTTT | |
| ATACTGGAAAGAATGGAAGAACGTCGAGTTGCCTAAGGAACTTCTTAAA | |
| AAGATTACACTGGCCCAGGACCACTTACTTGATAAGGAAACTATTTACC | |
| CATTTTACCACGGATTCGAAGAGTACTCATGGAACACGATGATTCTGGG | |
| ACTGTGTTCTGGTTCACTGAATAACAAACCGGCACTGTCCTTAATGACGT | |
| CCGATGAGGCTGATGAAATGCTGCTGCAACACTATATGAAAGCTGAGAA | |
| CATGGTGAATAACCTGCCGACCTGCTATGAATATTTGAAGCACATTCAC | |
| GACTTGAAAAACAAAAATTAA | |
| ATGGATAACCGTATTAAGACCGTCGTTATTCTGGGAGGTGGGACCGCGG | 19 |
| GGTGGATGACAGCAGCCTACCTTGGTAAGGCTCTTCAAAATACAGTGAA | |
| GATTGTGGTCTTAGAAGCTCCGACGATCCCTCGTATCGGGGTGGGAGAA | |
| GCAACTGTACCGAATTTACAACGCGCCTTCTTTGACTATTTGGGGATTCC | |
| GGAAGAAGAGTGGATGCGTGAATGTAACGCATCTTACAAAATGGCAGTG | |
| AAGTTTATCAATTGGCGCACCCCTGGCGAGGGTTCACCTGATCCGCGTAC | |
| TTTGGACGACGGCCACACAGACACTTTTCATCACCCCTTCGGTCTTCTTC | |
| CAAGTGCCGATCAGATTCCCCTGTCACATTACTGGGCTGCCAAACGCTTA | |
| CAAGGTGAGACAGATGAGAATTTCGATGAGGCGTGCTTTGCGGACACTG | |
| CCATCATGAATGCCAAAAAAGCTCCACGTTTTCTTGACATGCGCCGTGCG | |
| ACTAATTACGCGTGGCATTTCGACGCCTCTAAGGTAGCTGCTTTTCTGCG | |
| TAACTTTGCAGTCACAAAACAAGCGGTTGAGCATGTCGAAGACGAAATG | |
| ACCGAGGTTTTGACGGATGAGCGTGGGTTTATCACTGCTTTGCGTACGAA | |
| GTCCGGCCGTATTTTGCAAGGAGATCTTTTCGTCGACTGCTCGGGGTTTC | |
| GTGGACTGCTTATCAACAAAGCTATGGAGGAGCCCTTTATTGACATGTCC | |
| GACCATTTGTTGTGCAATAGCGCAGTCGCCACGGCTGTGCCACACGACG | |
| ATGAAAAAAATGGTGTGGAGCCGTACACAAGTTCCATCGCTATGGAAGC | |
| TGGATGGACCTGGAAGATTCCCATGCTGGGCCGTTTCGGGAGCGGTCAC | |
| GTGTACAGCGACCATTTCGCAACGCAAGACGAAGCGACCCTGGCATTCT | |
| CGAAATTATGGGGGTTAGACCCAGATAACACCGAATTCAACCACGTACG | |
| CTTCCGTGTGGGACGTAATCGCCGTGCATGGGTACGCAATTGTGTGTCAG | |
| TGGGTCTTGCAAGCTGCTTCGTGGAACCACTTGAAAGTAGTGGCATCTAT | |
| TTCATTTATGCCGCGATCCATATGTTAGCGAAGCACTTTCCGGACAAAAC | |
| TTTCGATAAGGTTTTAGTCGATCGCTTTAACCGTGAGATTGAGGAGATGT | |
| TCGATGACACACGCGATTTCTTGCAGGCTCACTACTATTTTAGTCCGCGC | |
| GTGGACACACCCTTCTGGCGTGCCAACAAGGAATTGAAGTTAGCGGATT | |
| CTATTAAGGACAAAGTCGAGACCTATCGCGCAGGACTGCCAGTGAATTT | |
| GCCTGTGACCGACGAAGGCACCTATTATGGAAACTTCGAGGCCGAGTTC | |
| CGTAATTTCTGGACCAACGGATCGTACTACTGTATCTTCGCCGGGCTTGG | |
| GCTGATGCCGCGCAACCCATTACCAGCTCTTGCTTACAAGCCACAAAGC | |
| ATCGCCGAAGCCGAGTTATTATTTGCTGACGTCAAACGCAAAGGCGACA | |
| CGCTTGTAGAATCGTTGCCTAGCACGTATGATCTGTTGCGTCAGCTTCAT | |
| GGGGCTTCCTGA | |
| ATGCTTAATAATGTCGTTATCGTAGGGGGAGGAACCGCTGGCTGGATGA | |
| CAGCCTCCTATTTAAAGGCTGCTTTCGGGGATCGCATTGACATCACTTTG | 20 |
| GTCGAATCGGGTCATATTGGCGCCGTTGGCGTTGGAGAGGCTACATTCTC | |
| TGACATTCGCCATTTTTTCGAATTCCTGGGATTAAAGGAGAAAGACTGG | |
| ATGCCGGCGTGTAATGCAACCTACAAGCTGGCCGTTCGCTTTGAGAACT | |
| GGCGCGAAAAGGGACACTATTTTTATCATCCGTTCGAGCAAATGCGTAG | |
| TGTCAACGGGTTCCCACTTACAGATTGGTGGTTAAAACAAGGACCGACA | |
| GACCGTTTTGATAAAGATTGTTTCGTAATGGCCAGTGTCATTGACGCCGG | |
| ACTTTCTCCCCGTCACCAAGATGGCACGCTGATTGATCAGCCATTCGACG | |
| AGGGAGCTGACGAGATGCAGGGTCTGACAATGTCTGAACATCAGGGTAA | |
| AACTCAATTCCCTTACGCGTATCAATTTGAGGCCGCATTGCTTGCGAAAT | |
| ACCTTACGAAATACTCCGTAGAGCGCGGGGTGAAGCACATCGTAGACGA | |
| CGTGCGCGAGGTATCACTTGACGACCGTGGATGGATCACAGGTGTACGT | |
| ACCGGAGAGCATGGCGATTTGACAGGCGATCTTTTTATTGACTGCACAG | |
| GATTCCGTGGTTTATTGTTAAACCAAGCCCTTGAAGAACCCTTTATCAGC | |
| TATCAGGATACGTTGCCAAATGACTCCGCTGTTGCTTTACAAGTACCGAT | |
| GGATATGGAGCGTCGCGGAATTTTACCTTGTACGACCGCCACGGCACAG | |
| GATGCTGGTTGGATTTGGACAATCCCATTAACTGGGCGCGTTGGAACCG | |
| GCTATGTCTATGCTAAAGACTACCTTAGCCCGGAGGAAGCAGAACGTAC | |
| ATTACGCGAGTTTGTGGGGCCTGCGGCAGCGGATGTGGAAGCTAATCAT | |
| ATTCGCATGCGTATTGGTCGTAGCCGTAACTCATGGGTGAAAAATTGTGT | |
| GGCGATCGGTTTGAGTAGCGGCTTTGTGGAACCGCTTGAGTCAACCGGT | |
| ATCTTTTTTATTCACCATGCGATTGAGCAATTGGTCAAGAACTTTCCGGC | |
| GGCTGATTGGAACAGTATGCACCGCGACCTGTATAATTCGGCGGTGTCG | |
| CATGTCATGGACGGGGTTCGTGAGTTTCTTGTATTGCACTATGTGGCGGC | |
| GAAGCGTAACGATACACAATATTGGCGTGATACCAAGACTCGTAAGATC | |
| CCTGATTCACTTGCAGAACGCATCGAGAAATGGAAGGTTCAACTGCCGG | |
| ATAGTGAGACGGTATATCCTTACTACCACGGGTTACCTCCGTACTCGTAT | |
| ATGTGCATCTTGCTGGGTATGGGTGGAATTGAGCTGAAGCCGTCGCCCG | |
| CGCTTGCTCTTGCCGACGGCGGGGCTGCGCAACGCGAATTTGAGCAAAT | |
| CCGTAACAAAACCCAACGTCTGACCGAGGTTTTGCCCAAAGCGTACGAC | |
| TATTTTACCCAACTGCGTTAA | |
| ATGAACACACGTAATCCGGACAAGGTGGTAATTGTCGGCGGTGGTACAG | |
| CAGGCTGGATGACGGCGTCTTACTTGAAAAAAGCATTTGGTGAGCGTGT | |
| GTCAGTTACACTTGTTGAGTCCGGCACTATCGGTACGGTGGGGGTGGGT | |
| GAGGCTACCTTTTCGGATATTCGCCACTTCTTTGAGTTCCTCGATCTGCGT | |
| GAAGAGGAGTGGATGCCGGCGTGCAATGCAACTTACAAGCTGGCGGTGC | |
| GCTTTCAAGATTGGCAGCGCCCAGGGCATCATTTTTATCATCCCTTCGAG | |
| CAGATGCGCTCGGTCGATGGGTTTCCTTTGACGGATTGGTGGCTGCAAA | |
| ACGGCCCAACCGATCGTTTTGATCGCGATTGCTTCGTGATGGCGAGCCTG | |
| TGCGATGCAGGACGGTCGCCTCGCTATCTTAATGGCAGTCTGCTTCAGCA | |
| GGAATTCGATGAACGCGCTGAAGAGCCTGCTGGTTTGACCATGAGTGAA | |
| CACCAGGGCAAAACACAATTCCCCTATGCATATCATTTTGAGGCGGCGT | |
| TGCTCGCGGAATTTCTGTCAGGTTATAGCAAAGATCGTGGCGTTAAGCA | |
| CGTGGTGGACGAAGTGCTGGAAGTGAAGCTGGATGATCGCGGCTGGATC | |
| TCTCACGTTGTCACGAAAGAACACGGCGACATTGGTGGCGACCTGTTTG | |
| TCGATTGCACGGGTTTTCGCGGCGTCCTGCTCAACCAGGCACTGGGGGTT | 21 |
| CCGTTTGTATCATACCAGGATACGCTCCCAAATGATTCGGCGGTCGCGCT | |
| GCAGGTGCCGCTTGACATGGAGGCTCGCGGAATTCCACCGTATACTCGG | |
| GCCACCGCAAAGGAAGCGGGATGGATTTGGACGATTCCACTCATTGGTC | |
| GTATCGGCACCGGCTACGTCTACGCCAAAGATTACTGCTCGCCAGAAGA | |
| GGCCGAGCGTACGCTGCGTGAATTCGTCGGTCCCGAAGCAGCGGATGTT | |
| GAGGCTAACCACATTCGCATGCGTATTGGCCGCAGCGAGCAAAGCTGGA | |
| AAAATAACTGTGTCGCCATTGGCCTCTCCAGCGGCTTTGTCGAACCGCTG | |
| GAGAGCACGGGTATTTTTTTTATTCATCATGCGATCGAGCAGCTGGTGAA | |
| ACACTTTCCGGCCGGCGATTGGCACCCGCAATTGCGTGCCGGCTACAAT | |
| AGTGCTGTGGCGAACGTTATGGACGGAGTGCGCGAATTCCTGGTTCTGC | |
| ATTATCTTGGCGCTGCGCGTAATGACACACGCTATTGGAAAGATACGAA | |
| GACGCGCGCAGTGCCGGACGCACTTGCCGAACGTATCGAGCGTTGGAAA | |
| GTGCAGCTGCCGGATTCGGAGAACGTCTTTCCGTACTATCATGGTTTACC | |
| ACCTTATAGTTATATGGCAATCCTGCTGGGTACAGGTGCAATCGGTCTGC | |
| GCCCGTCGCCGGCTTTGGCACTGGCGGACCCGGCGGCTGCTGAAAAGGA | |
| ATTTACCGCAATTCGCGATCGTGCGCGCTTTCTGGTCGATACCCTTCCAT | |
| CACAGTACGAATACTTTGCAGCCATGGGTCAACGTGTCTAA | |
| ATGAGTGGCAAGATTGATAAAATTTTGATCGTGGGCGGCGGTACCGCGG | 22 |
| GCTGGATGGCAGCTTCGTATTTGGGCAAGGCCTTGCAGGGAACTGCCGA | |
| TATCACCTTACTGCAGGCGCCCGACATCCCAACTCTGGGGGTAGGTGAG | |
| GCCACGATTCCTAATCTTCAGACCGCCTTTTTTGACTTCTTGGGCATTCCC | |
| GAGGATGAATGGATGCGTGAGTGTAATGCCAGTTACAAAGTGGCAATCA | |
| AATTTATTAACTGGCGCACAGCTGGCGAGGGGACTTCCGAAGCTCGCGA | |
| ATTAGATGGAGGCCCCGATCATTTCTACCATAGTTTCGGCCTGTTAAAGT | |
| ATCACGAGCAGATTCCCTTGAGTCACTACTGGTTTGACCGTAGTTATCGC | |
| GGAAAAACAGTGGAGCCGTTCGACTACGCCTGCTATAAAGAGCCAGTTA | |
| TCCTTGACGCCAACCGCTCACCACGTCGTCTGGATGGCTCCAAGGTTACG | |
| AATTACGCTTGGCACTTTGATGCGCATTTGGTTGCCGATTTTTTGCGCCG | |
| TTTCGCGACAGAGAAGTTGGGAGTTCGCCATGTGGAAGATCGTGTCGAG | |
| CATGTTCAGCGTGATGCCAATGGTAACATTGAGTCCGTTCGTACCGCCAC | |
| AGGTCGTGTCTTCGATGCGGACTTATTCGTTGACTGCTCTGGGTTCCGTG | |
| GTCTGCTGATTAATAAGGCAATGGAGGAGCCATTTTTGGATATGTCGGA | |
| CCATCTGCTTAATGATTCTGCGGTCGCCACCCAAGTACCGCACGATGACG | |
| ATGCTAATGGGGTGGAACCCTTTACGAGCGCAATCGCTATGAAGAGCGG | |
| ATGGACGTGGAAAATCCCTATGCTGGGACGCTTCGGGACTGGTTACGTT | |
| TATAGTTCGCGTTTTGCAACCGAAGACGAGGCGGTGCGTGAGTTCTGCG | |
| AGATGTGGCATTTAGACCCGGAGACGCAACCTCTGAACCGCATCCGTTT | |
| CCGCGTCGGCCGTAACCGCCGTGCCTGGGTCGGTAACTGCGTTAGCATT | |
| GGCACATCAAGTTGTTTCGTAGAACCACTGGAGTCAACAGGGATTTACT | |
| TCGTTTATGCGGCACTTTATCAACTGGTAAAGCATTTCCCTGATAAATCG | |
| CTTAACCCTGTTCTGACAGCCCGTTTCAATCGCGAGATTGAAACTATGTT | |
| CGACGACACCCGTGACTTTATCCAAGCACACTTCTACTTCTCGCCGCGCA | |
| CCGATACACCCTTCTGGCGCGCTAACAAGGAACTGCGCTTGGCAGATGG | |
| AATGCAAGAAAAAATTGACATGTACCGTGCAGGCATGGCTATTAATGCC | |
| CCTGCCTCGGACGACGCGCAGTTGTATTATGGCAACTTCGAAGAGGAAT | |
| TCCGCAATTTTTGGAACAATTCGAACTATTACTGTGTTTTAGCAGGATTA | |
| GGACTTGTTCCTGACGCGCCGTCTCCACGTCTTGCTCATATGCCTCAAGC | |
| AACAGAGAGTGTGGATGAAGTCTTCGGAGCAGTGAAAGATCGTCAACGT | |
| AATCTTTTAGAAACCCTTCCGAGTTTACATGAATTCTTACGTCAACAGCA | |
| TGGACGTTAA | |
| ATGAATAAACCGATCAAGAATATCGTGATCGTTGGGGGCGGCACGGCTG | 23 |
| GGTGGATGGCGGCGTCGTATCTGGTACGTGCGTTACAGCAACAAGCCAA | |
| CATCACCTTGATCGAATCCGCAGCGATTCCCCGCATCGGTGTAGGGGAA | |
| GCCACGATCCCGTCTCTGCAAAAGGTGTTTTTTGACTTTCTTGGCATTCC | |
| CGAGCGCGAGTGGATGCCTCAGGTGAACGGCGCCTTTAAAGCCGCTATT | |
| AAATTCGTCAACTGGCGCAAATCACCGGACCCCTCTCGCGACGATCACT | |
| TTTACCATTTATTCGGAAATGTACCTAATTGTGATGGTGTTCCACTTACG | |
| CATTACTGGTTGCGTAAACGTGAGCAGGGATTCCAACAACCGATGGAGT | |
| ACGCGTGCTATCCTCAGCCAGGCGCATTGGATGGAAAACTGGCACCTTG | |
| TTTATCTGACGGCACCCGCCAGATGTCACACGCGTGGCATTTCGACGCAC | |
| ATTTGGTTGCCGACTTCCTTAAACGCTGGGCCGTGGAGCGTGGTGTAAAT | |
| CGTGTGGTTGATGAGGTCGTAGATGTGCGCCTTAATAACCGCGGGTACA | |
| TCTCAAATCTGCTGACAAAGGAGGGACGTACGCTTGAAGCTGACCTGTT | |
| TATTGATTGTTCGGGTATGCGTGGTCTGCTTATCAATCAAGCTTTAAAAG | |
| AACCGTTTATCGACATGTCCGACTACTTGCTGTGCGATAGTGCGGTTGCA | |
| AGTGCGGTGCCTAATGATGATGCACGTGACGGGGTAGAGCCCTATACCT | |
| CATCGATCGCAATGAACTCAGGATGGACTTGGAAAATCCCGATGCTTGG | |
| TCGCTTTGGATCGGGCTACGTCTTCTCGTCCCACTTCACTTCTCGTGACCA | |
| GGCTACGGCCGACTTTCTGAAGTTATGGGGGTTGTCGGACAATCAGCCG | |
| TTAAATCAAATTAAGTTTCGTGTGGGGCGCAACAAGCGTGCCTGGGTGA | |
| ACAATTGCGTGTCAATCGGCCTTTCCTCCTGTTTCCTTGAGCCTTTAGAGT | |
| CTACGGGCATTTACTTCATCTACGCGGCACTTTACCAGCTTGTAAAACAC | |
| TTTCCTGATACATCTTTTGATCCCCGTCTTTCAGACGCCTTTAATGCGGAG | |
| ATCGTGCACATGTTCGATGACTGCCGCGATTTTGTGCAAGCGCATTATTT | |
| TACGACCTCGCGTGACGACACACCGTTCTGGCTGGCGAATCGCCACGAC | |
| CTGCGTCTTAGTGACGCAATTAAAGAGAAAGTGCAGCGTTACAAGGCCG | |
| GTCTTCCCCTTACAACCACCAGCTTCGACGACTCGACATATTACGAGACC | |
| TTCGACTATGAATTCAAGAATTTCTGGCTGAATGGGAATTACTATTGCAT | |
| CTTCGCTGGTCTGGGGATGTTGCCAGACCGTTCCCTTCCATTATTGCAGC | |
| ATCGCCCGGAAAGCATCGAAAAAGCTGAAGCGATGTTTGCCTCGATCCG | |
| CCGCGAGGCTGAACGCTTGCGTACTAGTCTGCCCACAAATTATGACTACT | |
| TGCGTAGTCTTCGTGACGGCGATGCCGGCCTTTCCCGCGGTCAACGTGGT | |
| CCCAAATTGGCTGCTCAAGAAAGTTTGTAA | |
| CCACTAGTCAGTTAACGggctgtcgacCTTTGAAAAGTTCGttTaca | 24 |
| gctagctcagtcctaggtaCAATtgtgagcgctcacaatttcacTGGCtgagcac | 25 |
| agctgtcaccggatgtgctttccggtctgatgagtccgtgaggacgaaacagcctctacaaataattttgtttaa | 26 |
| actagAAGTAAGCGAGGTACacaT | 27 |
| taATCCAAACCTGTTATATGTTAGCTGAGACTAGTTGGAAGTGTGGctgtcctc | 28 |
| aagcgttttagttcgtcggtcagtttcacctgatttacgtaaaaacccgcttcggcgggtttttgcttttggaggggcagaaa | |
| gatgaatgactgtc | |
| ATGAAGGTGCTTGTATTAGCCTTCCATCCGAATATGGAGCAAAGTGTCGT | 29 |
| GAATCGTGCATTCGCCGACACTCTTAAAGACGCGCCTGGCATTACGTTGC | |
| GTGATTTATATCAGGAATACCCAGACGAGGCCATCGACGTGGAAAAAGA | |
| ACAGAAACTGTGTGAGGAACACGACCGCATTGTTTTTCAATTTCCATTAT | |
| ATTGGTATAGCAGTCCCCCTCTGCTGAAAAAGTGGTTGGATCATGTTCTG | |
| CTGTATGGTTGGGCATACGGCACTAACGGTACTGCCTTACGCGGTAAGG | |
| AATTCATGGTGGCGGTCAGCGCAGGGGCTCCAGAGGAAGCGTACCAGGC | |
| TGGGGGATCGAACCATTATGCGATTAGTGAATTGTTGCGTCCATTTCAGG | |
| CTACCTCAAACTTTATTGGTACAACTTATCTTCCTCCTTATGTTTTTTATC | |
| AGGCCGGTACCGCCGGTAAATCTGAATTAGCAGAGGGCGCGACCCAGTA | |
| TCGTGAGCATGTGTTGAAGTCGTTTTGA | |
| ATGACAACCTTAAGCTGTAAAGTGACCTCGGTAGAAGCTATCACGGATA | 30 |
| CCGTATATCGTGTCCGCATCGTGCCAGACGCGGCCTTTTCTTTTCGTGCT | |
| GGTCAGTATTTGATGGTAGTGATGGATGAGCGCGACAAACGTCCGTTCT | |
| CAATGGCTTCGACGCCGGATGAAAAAGGGTTTATCGAGCTGCATATTGG | |
| CGCTTCTGAAATCAACCTTTACGCGAAAGCAGTCATGGACCGCATCCTC | |
| AAAGATCATCAAATCGTGGTCGACATTCCCCACGGAGAAGCGTGGCTGC | |
| GCGATGATGAAGAGCGTCCGATGATTTTGATTGCGGGCGGCACCGGGTT | |
| CTCTTATGCCCGCTCGATTTTGCTGACAGCGTTGGCGCGTAACCCAAACC | |
| GTGATATCACCATTTACTGGGGCGGGCGTGAAGAGCAGCATCTGTATGA | |
| TCTCTGCGAGCTTGAGGCGCTTTCGTTGAAGCATCCTGGTCTGCAAGTGG | |
| TGCCGGTGGTTGAACAACCGGAAGCGGGCTGGCGTGGGCGTACTGGCAC | |
| CGTGTTAACGGCGGTATTGCAGGATCACGGTACGCTGGCAGAGCATGAT | |
| ATCTATATTGCCGGACGTTTTGAGATGGCGAAAATTGCCCGCGATCTGTT | |
| TTGCAGTGAGCGTAATGCGCGGGAAGATCGCCTGTTTGGCGATGCGTTT | |
| GCATTTATC | |
| ATGAGTAACGCTACCGAGGAGCTTACCACAGTACGTGACGCGTGTGCGC | 31 |
| GCACCCTGGAGAATACAGCCCGTACGCTTCATCTGGGCGCATCGGGTAC | |
| CGAGTTTGTCGCTGCCTTCCGTGCTATGACGGACCACTGGGGCGCCGCAC | |
| GCCCGCATGACTTACCCTTGTCGGATGTCTCGCCTGACGGAAGTCCCGTT | |
| GAGTATGCAGTTGATCTTGGAGGACTTGCACCTGCACTGCAGTTCGCTAT | |
| GGAGCCACTTACTGCAGGAGTCCCTGCACGTGATCCTTTAGCCGCTCGCG | |
| CCATTATGCCCTTATTAGCGGGACGCTATGGTGCCGACGCGACCCGTTGG | |
| AGCGCTCTGGCTGACCGTTTACTTCCGGATGATGCCCATGGACCGCATGT | |
| TTCCATGTATGGCGCGGAGGTCCGCGCGGGTGCCCCTATCCGTTTCAAGG | |
| CTTGGTTCTACCTGAACGTCACAGGGCCTGACGGGGCGTTTAATCTGCTT | |
| TATAGCGCCCTGGAACGTATGGGTACTACACACCTTTGGCCTGTAGTCCA | |
| AGCCCATGTCCACCGCGCAGGTGAGGATGTACCATTCCTGCTTTCATTAG | |
| ATTTATCAGATGATCCAGCGGCCCGCGTCAAAGTATACTTTCGTCACTTT | |
| GCGGCAGACGTCGAAGAAGTCGCTGCCGTGCTGAAGGCTTATCCCGGCT | |
| TCGAACCGGGTGAAGTCCGTGCGTTTTGCAAGGTGATGATGGGAGGCCG | |
| TCGTCGTTTTTCCGACCAGCCGGCTGTTACATGCGTGTCTTTGTTAGACG | |
| CTCAGACGTTCGACCGTACTGCTGCCACGCTGTACGTTCCTTTGTGGACA | |
| TACGCGGAACACGATGGAGAAGTGCGCCAGCGTGTTCACCGCACTCTGG | |
| CAGCATGGCCTGAAGCGCTTTATCGCTACGACTCCGTCTTGGCTGGCATT | |
| GCCCATCGCGGCCTTGACGCAGGTACTGGCATCCATAACTATATCAGTTG | |
| GCAACCCGGTCGCACGCGTCCTCGCATGAAGGTGTATTTATCTCCGGAG | |
| ATGCACGATGTTACCCCACCGCCACTGGGGGTGTCACAGCAACATCACT | |
| TATCGGGGCAGACTACAGCTCGCGGCCGTACTGAGTAA | |
| ATGTGTCCATGCCCGCATTCACAAGCGCAGGGAGAGACGGACGGCGAA | 32 |
| GCGGAATGGCACAATGCCGCGTTAGACTTTACTCATGCCATGTCGTATG | |
| GTGATTATCTGAAGCTGGATAAAGTACTGGATGCACAGTTTCCACTTTCC | |
| CCCGATCACAATGAGATGCTTTTTATTATTCAACACCAGACCAGCGAATT | |
| ATGGATGAAACTGATGCTGCATGAGCTTCGTGCGGCCCGCGAGCACGTC | |
| AAAAGTGGGAAATTGGGTCCCGCATTGAAGATGCTGGCTCGTGTAAGCC | |
| GTATTTTTGACCAACTTGTTCATGCATGGGCTGTTTTGGCTACCATGACG | |
| CCCACAGAGTATAATACTATTCGCCCCTATCTTGGTCAATCTTCGGGGTT | |
| TCAAAGCTATCAATATCGCGAAATCGAGTTCATTTTAGGGAATAAGAAC | |
| GCTACACTGCTGAAGCCACATGCCCACCGTGCAGAGCTTCTTGCGGCTTT | |
| GGAGCAGGCGTTACATACACCTTCACTTTATGACGAGGCAATTCGTTTAA | |
| TGGCTGCTCAGGGATTACCGGTCTTCCAGGAACGTTTGGTACGTGACGC | |
| AGCTGCGGGAACGTGTTACGAAGCATCTGTAGAGGCCGCATGGCGTCAA | |
| GTTTACCAAACGCCAGAGCGCTACTGGGACCTGTACCAGCTTGCTGAAA | |
| AACTGATCGACCTGGAGGACTCATTCCGCCAATGGCGTTTTCGCCACGTT | |
| ACCACCGTTGAGCGCATTATTGGATTCAAACCGGGAACGGGTGGGACCG | |
| AAGGCGTTGGGTACCTTCGTTCCATGTTGGATACAATTCTGTTCCCGGAA | |
| CTGTGGCGTTTGCGCTCGAACCTTTGA | |
| ATGTTGCGCAAAGGAACTGTGGCTCTTATTAACCCCAATCAAATCCACCC | 33 |
| GCCGATCGCCCCCTATGCTTTAGACGTATTAACTACCGCGCTTGAAGCTT | |
| CCGGATTTGAGGCACACGTCCTTGACCTGACCTTTCATTTGGATGATTGG | |
| CGCCAGACGTTACGTGATTACTTCCGCGCAGAACGTCCACTTCTGGTGGG | |
| CGTCACGTGCCGCAACACGGATACTGTGTATGCTTTAGAGCAGCGCCCTT | |
| TTGTCGACGGATACAAAGCAGTCATCGACGAAGTTCGCCGCTTAACCGC | |
| TGCCCCCGTCGTAGCAGGCGGCGTGGGATTCTCCACAATGCCTTTTGCTC | |
| TGGTGGATTACTTCGGAATTGAGTACGGCGTAAAAGGCCCTGGCGAGAA | |
| GATCATTTGTGACTTAGCTCGTGCCTTAGCTGAGGGACGTAGTGCGGACC | |
| GCATTCACATTCCAGGTCTTTTAGTAAACCGCGGCCCGGGCAACGTCACC | |
| CGCGTAGCGCCACCTGCATTAGACCCGCGCGCAGCTCCGGCACCATCTA | |
| GCAGTCCTAGCCCATCGCCTGCACCGAGTTCCAGTTCAGCGCCTGTCCCG | |
| GTCCCCTTGTCCTTTGCGGCCGTCGGACATCATGAAAGTCGTGCTTGGCA | |
| GGCGGAGACAGAATTACCATACACTCGCCGTTCTGGAGAACCCTACAAG | |
| GTCGATAATCTTCGTTACTACCGCGAAGGCGGGCTGGGTAGTATCCTGA | |
| CAAAAAACGGGTGTGTATATAAATGCTCATTCTGCGTCGAGCCTGATGC | |
| CAAAGGCACGCAATTCGCCCGCCGTGGGATCACCGCAGTTGTGGACGAA | |
| ATGGAGGCTTTGACAGCGCAAGGTATCCACGATCTGCATACGACTGACA | |
| GTGAGTTTAATCTGTCAATCGCACATTCCAAAAATCTGTTACGTGAAATC | |
| GTTCGCCGTCGCGACCATGATGCGACCTCCCCGCTGCGCGACTTACGCTT | |
| ATGGGTATACTGCCAACCGAGCCCTTTCGATGAAGAGTTTGCAGAGCTG | |
| CTGGCTGCCGCAGGTTGTGCGGGCGTTAACATCGGAGCAGATCATACTC | |
| GTCCAGAAATGTTGGACGGTTGGAAGGTGACAGCCAAAGGTACACGCTA | |
| TTACGACTTCGCGGACACCGAACGTTTGGTACAATTGTGCCACCGTAATG | |
| GTATGTTGACTATGGTTGAAGCCTTATTCGGTATGCCCGGCGAAACCTTA | |
| GAAACTATGCGCGATTGTGTCGACCGCATGATGGAGTTAGATGCCACGG | |
| TTACTGGCTTTTCTCTGGGATTACGCCTTCTTCCATATATGGGTCTTGCAA | |
| AAAGCCTTGCAGAGCAGTGCGATGGAGTACGCACTGTCCGTGGTCTTCA | |
| AAGTAATAATGCTAGTGGCCCGATCGTGTTGAAACAACTTCACCAATGT | |
| GATGGCCCTATTGAGTATGAACGTCAATTTATGTTTGACGAGAGCGGTG | |
| ACTTTCGCTTGGTATGTTACTTCTCCCCCGATTTACCGGAAGCTCCGGGT | |
| ACAGCAGACAGCCCTGACGGGATTTGGCGTGCAAGTGTCGACTTCTTGT | |
| GGGACCGCATTCCGAAAAGTGAGCAGTACCGTGTTATGTTGCCCACGTT | |
| AAGCGGGTCCTCAGAAAATGACAACAATTACGCTGATAACCCTTTCTTG | |
| ACAAGTTTAAATCGTAAAGGGTACACAGGAGCATTTTGGGCGCACTGGC | |
| GCGATCGTGAAGCAATTATGTCAGGAGCTACTTTACCATTGGGCGAACT | |
| TGCTGAAGCGGTCCGTTAA | |
| ATGGAAAACTTTAAACATCTCCCTGAACCGTTCCGCATTCGTGTTATTGA | 34 |
| GCCAGTAAAACGTACCACTCGCGCTTATCGTGAAGAGGCAATTATTAAA | |
| TCCGGTATGAACCCGTTCCTGCTGGATAGCGAAGATGTTTTTATCGATTT | |
| ACTGACCGACAGCGGCACCGGGGCGGTGACGCAGAGCATGCAGGCTGC | |
| GATGATGCGCGGCGACGAAGCCTACAGCGGCAGTCGTAGCTACTATGCG | |
| TTAGCCGAGTCAGTGAAAAATATCTTTGGTTATCAATACACCATTCCGAC | |
| TCACCAGGGCCGTGGCGCAGAGCAAATCTATATTCCGGTACTGATTAAA | |
| AAACGCGAGCAGGAAAAAGGCCTGGATCGCAGCAAAATGGTGGCGTTC | |
| TCTAACTATTTCTTTGATACCACGCAGGGCCATAGCCAGATCAACGGCTG | |
| TACCGTGCGTAACGTCTATATCAAAGAAGCCTTCGATACGGGCGTGCGT | |
| TACGACTTTAAAGGCAACTTTGACCTTGAGGGATTAGAACGCGGTATTG | |
| AAGAAGTTGGTCCGAATAACGTGCCGTATATCGTTGCAACCATCACCAG | |
| TAACTCTGCAGGTGGTCAGCCGGTTTCACTGGCAAACTTAAAAGCGATG | |
| TACAGCATCGCGAAGAAATACGATATTCCGGTGGTAATGGACTCCGCGC | |
| GCTTTGCTGAAAACGCCTATTTCATCAAGCAGCGTGAAGCAGAATACAA | |
| AGACTGGACCATCGAGCAGATCACCCGCGAAACCTACAAATATGCCGAT | |
| ATGCTGGCGATGTCCGCCAAGAAAGATGCGATGGTGCCGATGGGCGGCC | |
| TGCTGTGCATGAAAGACGACAGCTTCTTTGATGTGTACACCGAGTGCAG | |
| AACCCTTTGCGTGGTGCAGGAAGGCTTCCCGACATATGGCGGCCTGGAA | |
| GGCGGCGCGATGGAGCGTCTGGCGGTAGGTCTGTATGACGGCATGAATC | |
| TCGACTGGCTGGCTTATCGTATCGCGCAGGTACAGTATCTGGTCGATGGT | |
| CTGGAAGAGATTGGCGTTGTCTGCCAGCAGGCGGGCGGTCACGCGGCAT | |
| TCGTTGATGCCGGTAAACTGTTGCCGCATATCCCGGCAGACCAGTTCCCG | |
| GCACAGGCGCTGGCCTGCGAGCTGTATAAAGTCGCCGGTATCCGTGCGG | |
| TAGAAATTGGCTCTTTCCTGTTAGGCCGCGATCCGAAAACCGGTAAACA | |
| ACTGCCATGCCCGGCTGAACTGCTGCGTTTAACCATTCCGCGCGCAACAT | |
| ATACTCAAACACATATGGACTTCATTATTGAAGCCTTTAAACATGTGAAA | |
| GAGAACGCGGCGAATATTAAAGGATTAACCTTTACGTACGAACCGAAAG | |
| TATTGCGTCACTTCACCGCAAAACTTAAAGAAGTTTAA | |
| ATGAAGGCGGCCAATGCTTCTAGCGCGGAGGCCTACCGTGTTCTGAGTC | 35 |
| GTGCCTTCCGTTTTGACAACGAGGATCAAAAGTTATGGTGGCACTCCACT | |
| GCACCAATGTTCGCTAAGATGTTAGAAACAGCAAACTATACCACCCCAT | |
| GTCAATATCAATACTTGATCACGTATAAAGAGTGTGTTATTCCTAGCTTA | |
| GGCTGCTACCCCACCAATTCAGCACCTCGTTGGCTTTCAATCCTTACACG | |
| TTATGGCACGCCTTTCGAGCTTTCTCTGAATTGCAGTAATTCAATCGTCC | |
| GCTATACCTTTGAGCCAATCAATCAGCACACTGGAACTGATAAAGATCC | |
| GTTCAATACGCACGCTATTTGGGAGAGCCTTCAGCATCTTTTACCATTAG | |
| AAAAATCTATCGACCTTGAGTGGTTCCGCCATTTTAAACATGATTTAACC | |
| TTGAACAGTGAAGAGAGCGCATTCTTGGCACACAATGATCGCTTAGTGG | |
| GCGGCACTATTCGCACGCAAAACAAACTTGCATTAGATTTAAAGGACGG | |
| ACGCTTTGCGCTGAAAACGTATATTTACCCGGCCCTGAAAGCCGTTGTAA | |
| CCGGCAAAACGATCCACGAATTAGTTTTTGGCTCAGTTCGTCGCTTGGCT | |
| GTGCGTGAGCCGCGTATCCTTCCTCCTTTAAACATGCTTGAGGAATATAT | |
| CCGTTCACGTGGTTCTAAGTCGACAGCAAGCCCGCGTCTTGTGAGCTGTG | |
| ATTTAACGTCACCAGCGAAATCGCGTATCAAAATCTACTTGTTGGAGCA | |
| GATGGTAAGTCTTGAGGCCATGGAAGATTTATGGACATTGGGTGGCCGC | |
| CGTCGTGATGCTTCAACCTTGGAAGGATTAAGCCTGGTCCGCGAATTGTG | |
| GGACCTGATTCAGTTGTCACCGGGCTTAAAATCCTATCCTGCCCCTTATT | |
| TACCCTTGGGTGTAATCCCAGATGAACGCTTACCTCTGATGGCGAACTTT | |
| ACGCTGCATCAAAATGATCCTGTACCCGAACCACAAGTGTATTTCACAA | |
| CATTTGGCATGAACGATATGGCAGTGGCGGATGCGCTTACAACTTTTTTC | |
| GAGCGTCGCGGGTGGAGTGAGATGGCCCGCACTTACGAAACAACGTTAA | |
| AATCATACTATCCACATGCGGATCACGACAAGCTGAACTATCTTCATGC | |
| ATACATTAGCTTCTCTTATCGTGACCGCACTCCGTATTTGTCTGTGTACTT | |
| GCAGTCATTTGAAACCGGTGACTGGGCGGTGGCTAATTTGTCAGAATCG | |
| AAAGTCAAGTGCCAAGACGCCGCTTGCCAGCCAACCAGCCTGCCCCCTG | |
| ATCTTTCTAAAACCGGAGTCTATTATTCTGGACTGCACTAA | |
| ATGTCTATTGGAGCGGAGATTGATAGTTTAGTACCTGCGCCACCGGGATT | 36 |
| AAACGGAACCGCTGCAGGGTACCCAGCTAAAACTCAAAAGGAGCTTTCG | |
| AATGGTGATTTCGATGCACACGACGGCCTGTCCCTTGCGCAACTTACTCC | |
| CTACGACGTCCTTACGGCGGCTTTGCCGTTACCTGCACCTGCTAGTAGCA | |
| CCGGCTTTTGGTGGCGCGAAACCGGACCCGTCATGTCTAAGCTTTTAGCT | |
| AAGGCGAACTATCCTTTGTATACGCATTATAAATATCTTATGCTGTATCA | |
| CACACACATCTTGCCGTTACTGGGACCGCGCCCACCCCTTGAGAATTCCA | |
| CTCACCCCTCGCCCTCTAACGCGCCATGGCGTTCATTCCTTACGGATGAC | |
| TTTACTCCGCTTGAACCAAGTTGGAATGTGAACGGGAATAGCGAGGCAC | |
| AGAGCACAATTCGCCTTGGTATTGAGCCCATTGGATTCGAGGCGGGCGC | |
| GGCGGCTGACCCTTTTAACCAAGCCGCGGTTACGCAGTTTATGCACAGCT | |
| ACGAGGCTACGGAAGTTGGCGCCACATTAACGTTATTCGAACACTTTCG | |
| TAATGATATGTTCGTTGGCCCAGAAACTTACGCAGCTCTTCGCGCAAAG | |
| ATCCCGGAAGGTGAGCACACAACACAATCATTCCTTGCATTCGACTTGG | |
| ATGCCGGACGTGTTACCACGAAAGCGTATTTCTTCCCTATTCTTATGTCT | |
| TTGAAAACGGGACAAAGCACCACGAAAGTGGTAAGTGACAGTATTCTGC | |
| ATTTAGCATTGAAGAGTGAGGTCTGGGGCGTCCAGACGATTGCCGCAAT | |
| GAGCGTGATGGAAGCGTGGATTGGATCATACGGCGGAGCTGCCAAGACT | |
| GAAATGATCTCGGTTGATTGTGTCAATGAGGCAGATTCCCGCATTAAGA | |
| TTTACGTCCGTATGCCACATACATCACTGCGCAAGGTCAAAGAAGCATA | |
| TTGTCTTGGGGGTCGCTTAACCGATGAGAATACGAAGGAGGGATTAAAG | |
| CTGTTAGACGAGCTGTGGCGTACGGTGTTCGGCATCGATGATGAGGACG | |
| CGGAATTACCTCAGAACTCCCACCGTACGGCTGGGACTATCTTTAATTTT | |
| GAGTTACGTCCGGGGAAGTGGTTTCCAGAACCTAAAGTTTACTTGCCTGT | |
| ACGTCACTATTGTGAGTCTGATATGCAGATCGCTTCCCGTTTACAGACTT | |
| TCTTCGGGCGTTTAGGCTGGCACAACATGGAAAAGGATTATTGTAAACA | |
| CTTGGAGGACTTGTTTCCCCACCATCCCCTTAGCTCTTCAACTGGCACAC | |
| ACACATTCCTTAGTTTTTCCTATAAAAAGCAGAAGGGGGTATACATGAC | |
| AATGTATTATAATCTTCGTGTATATTCGACTTAA | |
| ATGAGTCAGGTAATTAAGAAAAAACGCAACACATTCATGATCGGAACCG | 37 |
| AATACATCTTGAATAGTACACAACTTGAAGAGGCTATTAAGTCTTTTGTG | |
| CACGACTTCTGTGCAGAAAAGCACGAAATTCATGACCAGCCAGTTGTCG | |
| TTGAAGCCAAAGAGCACCAAGAGGACAAGATCAAACAAATTAAAATTC | |
| CCGAGAAAGGGCGTCCTGTAAACGAAGTAGTATCAGAGATGATGAACG | |
| AAGTGTACCGCTATCGTGGCGATGCTAATCACCCACGCTTCTTTTCTTTC | |
| GTGCCCGGACCGGCCAGTAGCGTCTCCTGGCTTGGCGACATTATGACGA | |
| GTGCTTATAACATCCACGCGGGAGGTTCCAAATTAGCGCCCATGGTCAA | |
| TTGTATCGAACAGGAAGTATTGAAGTGGTTAGCGAAACAAGTTGGGTTT | |
| ACGGAGAACCCCGGTGGGGTTTTCGTATCTGGTGGAAGTATGGCGAACA | |
| TTACCGCTTTAACTGCCGCCCGCGACAATAAGTTAACCGACATTAATCTG | |
| CATCTTGGCACCGCTTACATCTCGGACCAGACGCATTCCAGCGTCGCCAA | |
| GGGGCTTCGTATTATTGGGATCACCGACTCTCGTATCCGTCGCATTCCCA | |
| CGAACTCACATTTTCAAATGGATACCACCAAACTTGAGGAGGCAATTGA | |
| GACGGACAAGAAATCTGGGTATATTCCGTTCGTAGTAATTGGCACAGCG | |
| GGTACTACAAACACGGGAAGCATTGATCCGCTGACAGAGATTTCCGCGT | |
| TGTGTAAAAAACACGACATGTGGTTTCACATTGACGGGGCGTACGGCGC | |
| GTCCGTGTTATTGAGCCCAAAGTACAAAAGTTTGCTTACCGGGACAGGC | |
| TTGGCGGATTCTATCTCGTGGGACGCACACAAGTGGTTATTTCAGACGTA | |
| CGGGTGTGCGATGGTCTTGGTTAAGGACATTCGCAACTTATTTCACTCGT | |
| TCCATGTAAACCCTGAGTACTTGAAGGATCTGGAGAACGATATTGATAA | |
| CGTAAACACCTGGGACATTGGAATGGAATTGACCCGCCCAGCGCGTGGC | |
| CTGAAGCTGTGGCTGACTCTGCAGGTTTTGGGTTCAGACTTAATCGGCTC | |
| CGCGATCGAGCACGGATTCCAGTTAGCGGTGTGGGCAGAAGAAGCTTTG | |
| AATCCCAAAAAGGATTGGGAAATTGTGTCGCCCGCACAGATGGCTATGA | |
| TCAACTTTCGTTACGCACCAAAAGACCTTACAAAAGAAGAACAGGACAT | |
| CCTTAATGAAAAAATTTCCCATCGCATCTTAGAATCCGGATACGCAGCG | |
| ATCTTCACCACAGTGCTTAACGGTAAAACAGTGCTGCGTATCTGTGCGAT | |
| TCACCCAGAGGCCACTCAAGAAGATATGCAGCATACCATTGATCTGTTA | |
| GATCAATACGGGCGTGAGATTTACACCGAAATGAAGAAAGCCTGA | |
| ATGTATGATCATTTTAACAGCCCCTCAATTGATATTTTATATGATTACGG | 38 |
| TCCCTTTTTGAAGAAATGTGAGATGACAGGCGGGATCGGGTCGTATTCT | |
| GCAGGCACACCAACGCCGCGCGTAGCCATTGTTGGGGCTGGTATCAGCG | |
| GATTGGTCGCCGCCACTGAATTATTACGCGCGGGGGTTAAAGACGTGGT | |
| TTTATACGAATCGCGCGATCGTATCGGAGGCCGTGTTTGGTCACAGGTGT | |
| TCGATCAGACTCGTCCTCGTTATATTGCCGAAATGGGAGCAATGCGTTTT | |
| CCACCATCTGCTACAGGTTTGTTCCATTACTTGAAAAAATTTGGAATCTC | |
| TACTAGCACTACTTTTCCCGACCCCGGGGTAGTAGACACGGAATTGCATT | |
| ATCGCGGGAAGCGCTACCACTGGCCAGCCGGGAAAAAGCCTCCCGAGCT | |
| GTTCCGTCGCGTGTACGAAGGATGGCAATCCTTATTGAGCGAGGGTTAC | |
| CTGCTGGAAGGGGGCTCATTGGTTGCTCCCCTGGATATCACCGCAATGTT | |
| AAAGTCTGGCCGTCTGGAAGAGGCGGCGATTGCGTGGCAAGGATGGTTA | |
| AACGTCTTTCGTGACTGTTCGTTTTACAACGCTATTGTCTGTATCTTTACT | |
| GGTCGCCATCCTCCTGGGGGAGATCGTTGGGCGCGCCCTGAGGATTTCG | |
| AGTTATTCGGCTCGTTGGGGATTGGATCCGGGGGTTTCTTGCCGGTCTTC | |
| CAAGCAGGTTTTACCGAGATCTTGCGCATGGTAATTAATGGTTATCAATC | |
| AGACCAGCGCCTGATTCCCGATGGGATCTCATCCTTAGCCGCGCGTTTGG | |
| CAGACCAGTCTTTCGACGGAAAGGCACTTCGTGACCGCGTATGTTTCTCT | |
| CGTGTAGGACGCATCTCACGTGAAGCGGAAAAGATCATTATTCAAACCG | |
| AAGCCGGAGAGCAACGTGTGTTCGACCGTGTTATTGTTACTTCTTCTAAC | |
| CGTGCTATGCAGATGATTCATTGTTTGACTGATTCCGAGTCATTCCTTTCC | |
| CGTGATGTAGCACGTGCTGTCCGTGAGACCCATTTGACAGGATCTTCTAA | |
| ATTGTTTATTCTTACCCGCACTAAATTTTGGATCAAGAATAAATTGCCGA | |
| CGACAATTCAATCTGACGGCCTTGTTCGCGGGGTGTACTGTCTTGACTAT | |
| CAGCCAGACGAGCCGGAGGGTCATGGCGTTGTCCTTCTTTCGTATACTTG | |
| GGAAGACGATGCTCAAAAGATGTTAGCCATGCCTGATAAGAAGACCCGC | |
| TGTCAGGTTCTTGTTGATGATCTGGCAGCCATCCATCCCACTTTTGCTAG | |
| TTATCTGCTGCCGGTTGACGGCGACTATGAGCGTTACGTATTGCATCATG | |
| ACTGGCTTACTGACCCGCATAGTGCCGGCGCCTTCAAGTTGAATTACCCA | |
| GGGGAAGACGTATATTCGCAACGCCTGTTTTTCCAGCCGATGACAGCGA | |
| ATTCGCCTAACAAAGACACGGGCTTGTACTTAGCGGGCTGTTCTTGTTCA | |
| TTTGCGGGTGGGTGGATCGAGGGCGCGGTCCAAACGGCCCTGAACAGTG | |
| CTTGTGCCGTCCTGCGCTCCACAGGTGGTCAGCTTAGTAAAGGCAACCC | |
| GCTTGACTGTATTAACGCATCTTACCGCTATTAA | |
| ATGAATACATTTACGTCCAATTCCTCCGATTTAACAACCACGGCGACGG | 39 |
| AAACCTCCTCATTTTCAACCCTTTACTTGCTGAGTACCCTTCAGGCTTTCG | |
| TAGCAATCACTCTGGTTATGCTTCTGAAGAAGTTAATGACAGACCCAAA | |
| TAAGAAAAAACCGTACCTTCCACCCGGCCCAACAGGGTGGCCGATCATT | |
| GGGATGATCCCAACAATGTTGAAGAGTCGCCCCGTGTTTCGCTGGTTGC | |
| ACAGTATTATGAAGCAACTGAATACAGAGATTGCATGCGTAAAATTAGG | |
| TAACACACACGTAATTACAGTAACGTGCCCAAAGATCGCACGCGAGATC | |
| TTAAAACAGCAAGATGCACTTTTTGCTTCTCGCCCACTTACGTACGCTCA | |
| GAAGATTTTATCAAATGGATACAAGACATGCGTTATCACACCGTTTGGC | |
| GACCAGTTCAAAAAGATGCGTAAGGTTGTGATGACAGAATTGGTGTGTC | |
| CAGCCCGTCATCGCTGGCTTCATCAGAAACGTAGCGAGGAGAACGACCA | |
| CTTAACAGCGTGGGTCTACAATATGGTCAAAAATTCGGGGTCAGTTGAT | |
| TTTCGTTTTATGACCCGTCACTATTGCGGGAACGCCATTAAAAAGCTGAT | |
| GTTTGGAACTCGTACATTTAGTAAGAACACAGCGCCTGATGGTGGACCT | |
| ACAGTGGAGGACGTTGAACACATGGAGGCCATGTTTGAGGCGTTAGGTT | |
| TTACTTTCGCTTTCTGCATCTCCGACTATCTTCCCATGCTGACGGGTTTGG | |
| ACTTGAACGGGCATGAAAAGATCATGCGTGAGTCATCGGCCATTATGGA | |
| CAAGTATCACGACCCAATTATCGACGAGCGTATCAAGATGTGGCGTGAA | |
| GGAAAGCGCACGCAAATTGAAGATTTCTTAGATATCTTTATCAGCATCA | |
| AAGACGAACAAGGGAATCCTTTATTAACAGCCGACGAAATTAAACCGAC | |
| TATTAAAGAGTTGGTGATGGCAGCCCCAGACAACCCCTCTAACGCCGTA | |
| GAGTGGGCGATGGCGGAAATGGTGAACAAACCAGAGATCCTGCGTAAA | |
| GCCATGGAAGAAATTGATCGCGTCGTCGGAAAAGAGCGCTTGGTCCAAG | |
| AATCTGATATCCCGAAGTTGAATTATGTTAAGGCGATCCTGCGTGAAGC | |
| CTTCCGTTTACATCCGGTTGCTGCTTTCAATTTACCGCATGTAGCCTTAAG | |
| CGATACAACAGTCGCGGGATACCACATCCCCAAAGGTTCCCAGGTGTTG | |
| TTGTCACGCTATGGACTGGGTCGCAACCCTAAGGTGTGGGCCGATCCGTT | |
| GTGTTTCAAGCCAGAGCGTCACTTGAACGAATGCTCAGAAGTCACGTTA | |
| ACTGAGAATGATTTACGCTTTATCTCTTTCTCTACAGGAAAACGCGGTTG | |
| CGCTGCCCCCGCCCTGGGGACTGCCTTAACCACTATGATGCTTGCTCGCC | |
| TTCTTCAAGGGTTCACCTGGAAACTGCCCGAGAATGAAACCCGTGTGGA | |
| ACTGATGGAAAGTTCCCATGACATGTTTCTGGCCAAGCCACTTGTCATGG | |
| TCGGAGATTTGCGTTTGCCCGAACATTTATATCCCACTGTTAAATAA | |
| ATGGGATCCTCCCACCACCATCATCATCATAGTTCCGGTTTAGTTCCTAG | 40 |
| AGGATCACATATGATGAGACAAATAGAAATCGAGTGGGTCCAGCCTGGT | |
| ATTACTGTTACAGCAGACCTAAGTTGGGAAAGAAACCCAGAGCTTGCAG | |
| AGTTATTATGGACTGGACTACTACCATATAACAGTTTACAGAACCATGC | |
| ACTAGTGTCCGGTAACCACCTGTACCACTTGATAGCAGACCCCCGTTTAG | |
| TGTATACTGAGGCCCGTTACAAGGAGGATAGAACTAAGTCCCCCGATGG | |
| CACTGTCTTCTTAAGCCAGCTTCAGCATCTAGCAGTGAAGTATGGACCCC | |
| TAACAGAATACCTGCCAGCAGCACCAGTTGGCTCAGTGGTGCCAGAGGA | |
| TATTGACGCATTGAGAGAGGCAGGAAGAGCCTGTTGGAAGGCAGCTTGG | |
| GAAACAAAACAACCCATCGAAGTCAGGGTCCGTAGAAAAGGCGAAGCT | |
| GTAACCGATTTTGCTCTACCTAGGACTCCTCCAGTTGATCATCCTGGTGT | |
| CCAAAAGCTAGTTGAGGAAATACAAGACGAGACTGAAAGAGTGTGGAT | |
| AACTCCCCCAGCTGAGATCGTAGACATGCACCAAGGAAGAATTGCAAGC | |
| AGAGCCGGTAGCTACGATCAATATTTCAGCACTCTGGTATTTTTGAATGG | |
| AGAGGTCAGGCCTCTTGGATACTGCGCCCTAAACGGTCTTTTAAAGATTT | |
| GCCGTACAACTGATCTGACTCTAAACGATTTGAAGCGTATTACTCCAACT | |
| TTTATAAAGACTCCCGCAGAATTTTTGGGTTACACCGGTCTGGACACACT | |
| TTGGAGGTTCACACAGCAGGTCCTGACTTTATTACCAGATGTCGAAACC | |
| AGAGAACAGTATTTTGCACTTGTTAACGCACTGGCACTGTATGCCAACAT | |
| GTTGAATACTTGGAACCTACACTTTTTTCCCTGGCAGCATGGTACCGATT | |
| ACAGATACCTTGATGCATAA | |
| gctagctcagtcctaggtaCAATtacagccatcgtacgagcccTGGCtgagcac | 41 |
| actagCTGCCGCAGACCCGCacaT | 42 |
| ATGCAAACACAAAAACCGACTCTCGAACTGCTAACCTGCGAAGGCGCTT | 43 |
| ATCGCGACAATCCCACCGCGCTTTTTCACCAGTTGTGTGGGGATCGTCCG | |
| GCAACGCTGCTGCTGGAATTCGCAGATATCGACAGCAAAGATGATTTAA | |
| AAAGCCTGCTGCTGGTAGACAGTGCGCTGCGCATTACAGCTTTAGGTGA | |
| CACTGTCACAATCCAGGCACTTTCCGGCAACGGCGAAGCCCTCCTGGCA | |
| CTACTGGATAACGCCCTGCCTGCGGGTGTGGAAAGTGAACAATCACCAA | |
| ACTGCCGTGTGCTGCGCTTCCCCCCTGTCAGTCCACTGCTGGATGAAGAC | |
| GCCCGCTTATGCTCCCTTTCGGTTTTTGACGCTTTCCGTTTATTGCAGAAT | |
| CTGTTGAATGTACCGAAGGAAGAACGAGAAGCCATGTTCTTCGGCGGCC | |
| TGTTCTCTTATGACCTTGTGGCGGGATTTGAAGATTTACCGCAACTGTCA | |
| GCGGAAAATAACTGCCCTGATTTCTGTTTTTATCTCGCTGAAACGCTGAT | |
| GGTGATTGACCATCAGAAAAAAAGCACCCGTATTCAGGCCAGCCTGTTT | |
| GCTCCGAATGAAGAAGAAAAACAACGTCTCACTGCTCGCCTGAACGAAC | |
| TACGTCAGCAACTGACCGAAGCCGCGCCGCCGCTGCCAGTGGTTTCCGT | |
| GCCGCATATGCGTTGTGAATGTAATCAGAGCGATGAAGAGTTCGGTGGC | |
| GTAGTGCGTTTGTTGCAAAAAGCGATTCGCGCTGGAGAAATTTTCCAGG | |
| TGGTGCCATCTCGCCGTTTCTCTCTGCCCTGCCCGTCACCGCTGGCGGCC | |
| TATTACGTGCTGAAAAAGAGTAATCCCAGCCCGTACATGTTTTTTATGCA | |
| GGATAATGATTTCACCCTATTTGGCGCGTCGCCGGAAAGCTCGCTCAAGT | |
| ATGATGCCACCAGCCGCCAGATTGAGATCTACCCGATTGCCGGAACACG | |
| CCCACGCGGTCGTCGCGCCGATGGTTCACTGGACAGAGATCTCGACAGC | |
| CGTATTGAACTGGAAATGCGTACCGATCATAAAGAGCTGTCTGAACATC | |
| TGATGCTGGTTGATCTCGCCCGTAATGATCTGGCACGCATTTGCACCCCC | |
| GGCAGCCGCTACGTCGCCGATCTCACCAAAGTTGACCGTTATTCCTATGT | |
| GATGCACCTCGTCTCTCGCGTAGTCGGCGAACTGCGTCACGATCTTGACG | |
| CCCTGCACGCTTATCGCGCCTGTATGAATATGGGGACGTTAAGCGGTGC | |
| GCCGAAAGTACGCGCTATGCAGTTAATTGCCGAGGCGGAAGGTCGTCGC | |
| CGCGGCAGCTACGGCGGCGCGGTAGGTTATTTCACCGCGCATGGCGATC | |
| TCGACACCTGCATTGTGATCCGCTCGGCGCTGGTGGAAAACGGTATCGC | |
| CACCGTGCAAGCGGGTGCTGGTGTAGTCCTTGATTCTGTTCCGCAGTCGG | |
| AAGCCGACGAAACCCGTAACAAAGCCCGCGCTGTACTGCGCGCTATTGC | |
| CACCGCGCATCATGCACAGGAGACTTTCTGATGGCTGACATTCTGCTGCT | |
| CGATAATATCGACTCTTTTACGTACAACCTGGCAGATCAGTTGCGCAGCA | |
| ATGGGCATAACGTGGTGATTTACCGCAACCATATTCCGGCGCAAACCTT | |
| AATTGAACGCCTGGCGACCATGAGCAATCCGGTGCTGATGCTTTCTCCTG | |
| GCCCCGGTGTGCCGAGCGAAGCCGGTTGTATGCCGGAACTCCTCACCCG | |
| CTTGCGTGGCAAGCTGCCCATTATTGGCATTTGCCTCGGACATCAGGCGA | |
| TTGTCGAAGCTTACGGGGGCTATGTCGGTCAGGCGGGCGAAATTCTCCA | |
| CGGTAAAGCCTCCAGCATTGAACATGACGGTCAGGCGATGTTTGCCGGA | |
| TTAACAAACCCGCTGCCGGTGGCGCGTTATCACTCGCTGGTTGGCAGTA | |
| ACATTCCGGCCGGTTTAACCATCAACGCCCATTTTAATGGCATGGTGATG | |
| GCAGTACGTCACGATGCGGATCGCGTTTGTGGATTCCAGTTCCATCCGGA | |
| ATCCATTCTCACCACCCAGGGCGCTCGCCTGCTGGAACAAACGCTGGCC | |
| TGGGCGCAGCAGAAACTAGAGCCAGCCAACACGCTGCAACCGATTCTGG | |
| AAAAACTGTATCAGGCGCAGACGCTTAGCCAACAAGAAAGCCACCAGCT | |
| GTTTTCAGCGGTGGTGCGTGGCGAGCTGAAGCCGGAACAACTGGCGGCG | |
| GCGCTGGTGAGCATGAAAATTCGCGGTGAGCACCCGAACGAGATCGCCG | |
| GGGCAGCAACCGCGCTACTGGAAAACGCAGCGCCGTTCCCGCGCCCGGA | |
| TTATCTGTTTGCTGATATCGTCGGTACTGGCGGTGACGGCAGCAACAGTA | |
| TCAATATTTCTACCGCCAGTGCGTTTGTCGCCGCGGCCTGTGGGCTGAAA | |
| GTGGCGAAACACGGCAACCGTAGCGTCTCCAGTAAATCTGGTTCGTCCG | |
| ATCTGCTGGCGGCGTTCGGTATTAATCTTGATATGAACGCCGATAAATCG | |
| CGCCAGGCGCTGGATGAGTTAGGTGTATGTTTCCTCTTTGCGCCGAAGTA | |
| TCACACCGGATTCCGCCACGCGATGCCGGTTCGCCAGCAACTGAAAACC | |
| CGCACCCTGTTCAATGTGCTGGGGCCATTGATTAACCCGGCGCATCCGCC | |
| GCTGGCGTTAATTGGTGTTTATAGTCCGGAACTGGTGCTGCCGATTGCCG | |
| AAACCTTGCGCGTGCTGGGGTATCAACGCGCGGCGGTGGTGCACAGCGG | |
| CGGGATGGATGAAGTTTCATTACACGCGCCGACAATCGTTGCCGAACTG | |
| CATGACGGCGAAATTAAAAGCTATCAGCTCACCGCAGAAGACTTTGGCC | |
| TGACACCCTACCACCAGGAGCAACTGGCAGGCGGAACACCGGAAGAAA | |
| ACCGTGACATTTTAACACGTTTGTTACAAGGTAAAGGCGACGCCGCCCA | |
| TGAAGCAGCCGTCGCTGCGAACGTCGCCATGTTAATGCGCCTGCATGGC | |
| CATGAAGATCTGCAAGCCAATGCGCAAACCGTTCTTGAGGTACTGCGCA | |
| GTGGTTCCGCTTACGACAGAGTCACCGCACTGGCGGCACGAGGGTAAAT | |
| GATGCAAACCGTTTTAGCGAAAATCGTCGCAGACAAGGCGATTTGGGTA | |
| GAAGCCCGCAAACAGCAGCAACCGCTGGCCAGTTTTCAGAATGAGGTTC | |
| AGCCGAGCACGCGACATTTTTATGATGCGCTACAGGGTGCGCGCACGGC | |
| GTTTATTCTGGAGTGCAAGAAAGCGTCGCCGTCAAAAGGCGTGATCCGT | |
| GATGATTTCGATCCAGCACGCATTGCCGCCATTTATAAACATTACGCTTC | |
| GGCAATTTCGGTGCTGACTGATGAGAAATATTTTCAGGGGAGCTTTAATT | |
| TCCTCCCCATCGTCAGCCAAATCGCCCCGCAGCCGATTTTATGTAAAGAC | |
| TTCATTATCGACCCTTACCAGATCTATCTGGCGCGCTATTACCAGGCCGA | |
| TGCCTGCTTATTAATGCTTTCAGTACTGGATGACGACCAATATCGCCAGC | |
| TTGCCGCCGTCGCTCACAGTCTGGAGATGGGGGTGCTGACCGAAGTCAG | |
| TAATGAAGAGGAACAGGAGCGCGCCATTGCATTGGGAGCAAAGGTCGTT | |
| GGCATCAACAACCGCGATCTGCGTGATTTGTCGATTGATCTCAACCGTAC | |
| CCGCGAGCTTGCGCCGAAACTGGGGCACAACGTGACGGTAATCAGCGAA | |
| TCCGGCATCAATACTTACGCTCAGGTGCGCGAGTTAAGCCACTTCGCTAA | |
| CGGTTTTCTGATTGGTTCGGCGTTGATGGCCCATGACGATTTGCACGCCG | |
| CCGTGCGCCGGGTGTTGCTGGGTGAGAATAAAGTATGTGGCCTGACGCG | |
| TGGGCAAGATGCTAAAGCAGCTTATGACGCGGGCGCGATTTACGGTGGG | |
| TTGATTTTTGTTGCGACATCACCGCGTTGCGTCAACGTTGAACAGGCGCA | |
| GGAAGTGATGGCTGCGGCACCGTTGCAGTATGTTGGCGTGTTCCGCAAT | |
| CACGATATTGCCGATGTGGTGGACAAAGCTAAGGTGTTATCGCTGGCGG | |
| CAGTGCAACTGCATGGTAATGAAGAACAGCTGTATATCGATACGCTGCG | |
| TGAAGCTCTGCCAGCACATGTTGCCATCTGGAAAGCATTAAGCGTCGGT | |
| GAAACCCTGCCCGCCCGCGAGTTTCAGCACGTTGATAAATATGTTTTAGA | |
| CAACGGCCAGGGTGGAAGCGGGCAACGTTTTGACTGGTCACTATTAAAT | |
| GGTCAATCGCTTGGCAACGTTCTGCTGGCGGGGGGCTTAGGCGCAGATA | |
| ACTGCGTGGAAGCGGCACAAACCGGCTGCGCCGGACTTGATTTTAATTC | |
| TGCTGTAGAGTCGCAACCGGGCATCAAAGACGCACGTCTTTTGGCCTCG | |
| GTTTTCCAGACGCTGCGCGCATATTAAGGAAAGGAACAATGACAACATT | |
| ACTTAACCCCTATTTTGGTGAGTTTGGCGGCATGTACGTGCCACAAATCC | |
| TGATGCCTGCTCTGCGCCAGCTGGAAGAAGCTTTTGTCAGTGCGCAAAA | |
| AGATCCTGAATTTCAGGCTCAGTTCAACGACCTGCTGAAAAACTATGCC | |
| GGGCGTCCAACCGCGCTGACCAAATGCCAGAACATTACAGCCGGGACGA | |
| ACACCACGCTGTATCTCAAGCGTGAAGATTTGCTGCACGGCGGCGCGCA | |
| TAAAACTAACCAGGTGCTGGGGCAGGCGTTGCTGGCGAAGCGGATGGGT | |
| AAAACCGAAATCATCGCCGAAACCGGTGCCGGTCAGCATGGCGTGGCGT | |
| CGGCCCTTGCCAGCGCCCTGCTCGGCCTGAAATGCCGTATTTATATGGGT | |
| GCCAAAGACGTTGAACGCCAGTCGCCTAACGTTTTTCGTATGCGCTTAAT | |
| GGGTGCGGAAGTGATCCCGGTGCATAGCGGTTCCGCGACGCTGAAAGAT | |
| GCCTGTAACGAGGCGCTGCGCGACTGGTCCGGTAGTTACGAAACCGCGC | |
| ACTATATGCTGGGCACCGCAGCTGGCCCGCATCCTTATCCGACCATTGTG | |
| CGTGAGTTTCAGCGGATGATTGGCGAAGAAACCAAAGCGCAGATTCTGG | |
| AAAGAGAAGGTCGCCTGCCGGATGCCGTTATCGCCTGTGTTGGCGGCGG | |
| TTCGAATGCCATCGGCATGTTTGCTGATTTCATCAATGAAACCAACGTCG | |
| GCCTGATTGGTGTGGAGCCAGGTGGTCACGGTATCGAAACTGGCGAGCA | |
| CGGCGCACCGCTAAAACATGGTCGCGTGGGTATCTATTTCGGTATGAAA | |
| GCGCCGATGATGCAAACCGAAGACGGGCAGATTGAAGAATCTTACTCCA | |
| TCTCCGCCGGACTGGATTTCCCGTCTGTCGGCCCACAACACGCGTATCTT | |
| AACAGCACTGGACGCGCTGATTACGTGTCTATTACCGATGATGAAGCCC | |
| TTGAAGCCTTCAAAACGCTGTGCCTGCACGAAGGGATCATCCCGGCGCT | |
| GGAATCCTCCCACGCCCTGGCCCATGCGTTGAAAATGATGCGCGAAAAC | |
| CCGGATAAAGAGCAGCTACTGGTGGTTAACCTTTCCGGTCGCGGCGATA | |
| AAGACATCTTCACCGTTCACGATATTTTGAAAGCACGAGGGGAAATCTG | |
| ATGGAACGCTACGAATCTCTGTTTGCCCAGTTGAAGGAGCGCAAAGAAG | |
| GCGCATTCGTTCCTTTCGTCACGCTCGGTGATCCGGGCATTGAGCAGTCA | |
| TTGAAAATTATCGATACGCTAATTGAAGCCGGTGCTGACGCGCTGGAGT | |
| TAGGTATCCCCTTCTCCGACCCACTGGCGGATGGCCCGACGATTCAAAA | |
| CGCCACTCTGCGCGCCTTTGCGGCAGGTGTGACTCCGGCACAATGTTTTG | |
| AAATGCTGGCACTGATTCGCCAGAAACACCCGACCATTCCCATTGGCCT | |
| GTTGATGTATGCCAATCTGGTGTTTAACAAAGGCATTGATGAGTTTTATG | |
| CCCAGTGCGAAAAAGTCGGCGTCGATTCGGTGCTGGTTGCCGATGTGCC | |
| AGTTGAAGAGTCCGCGCCCTTCCGCCAGGCCGCGTTGCGTCATAATGTC | |
| GCACCTATCTTCATCTGCCCGCCAAATGCCGATGACGACCTGCTGCGCCA | |
| GATAGCCTCTTACGGTCGTGGTTACACCTATTTGCTGTCACGAGCAGGCG | |
| TGACCGGCGCAGAAAACCGCGCCGCGTTACCCCTCAATCATCTGGTTGC | |
| GAAGCTGAAAGAGTACAACGCTGCACCTCCATTGCAGGGATTTGGTATT | |
| TCCGCCCCGGATCAGGTAAAAGCAGCGATTGATGCAGGAGCTGCGGGCG | |
| CGATTTCTGGTTCGGCCATTGTTAAAATCATCGAGCAACATATTAATGAG | |
| CCAGAGAAAATGCTGGCGGCACTGAAAGTTTTTGTACAACCGATGAAAG | |
| CGGCGACGCGCAGTTAA | |
| ttacattaattgcgttgcgctcatttacggctagctcagtcctaggtactatgctagc | 44 |
| TCTAGAGTCACACAGGAAAGTACTAGATG | 45 |
| TCTAGAGAAAGAGGAGAAATACTAG | 46 |
| ATGGCAAAGGTATCGCTGGAGAAAGACAAGATTAAGTTTCTGCTGGTAG | 47 |
| AAGGCGTGCACCAAAAGGCGCTGGAAAGCCTTCGTGCAGCTGGTTACAC | |
| CAACATCGAATTTCACAAAGGCGCGCTGGATGATGAACAATTAAAAGAA | |
| TCCATCCGCGATGCCCACTTCATCGGCCTGCGATCCCGTACCCATCTGAC | |
| TGAAGACGTGATCAACGCCGCAGAAAAACTGGTCGCTATTGGCTGTTTC | |
| TGTATCGGAACAAACCAGGTTGATCTGGATGCGGCGGCAAAGCGCGGGA | |
| TCCCGGTATTTAACGCACCGTTCTCAAATACGCGCTCTGTTGCGGAGCTG | |
| GTGATTGGCGAACTGCTGCTGCTATTGCGCGGCGTGCCGGAAGCCAATG | |
| CTAAAGCGCACCGTGGCGTGTGGAACAAACTGGCGGCGGGTTCTTTTGA | |
| AGCGCGCGGCAAAAAGCTGGGTATCATCGGCTACGGTCATATTGGTACG | |
| CAATTGGGCATTCTGGCTGAATCGCTGGGAATGTATGTTTACTTTTATGA | |
| TATTGAAAATAAACTGCCGCTGGGCAACGCCACTCAGGTACAGCATCTT | |
| TCTGACCTGCTGAATATGAGCGATGTGGTGAGTCTGCATGTACCAGAGA | |
| ATCCGTCCACCAAAAATATGATGGGCGCGAAAGAAATTTCACTAATGAA | |
| GCCCGGCTCGCTGCTGATTAATGCTTCGCGCGGTACTGTGGTGGATATTC | |
| CGGCGCTGTGTGATGCGCTGGCGAGCAAACATCTGGCGGGGGCGGCAAT | |
| CGACGTATTCCCGACGGAACCGGCGACCAATAGCGATCCATTTACCTCT | |
| CCGCTGTGTGAATTCGACAACGTCCTTCTGACGCCACACATTGGCGGTTC | |
| GACTCAGGAAGCGCAGGAGAATATCGGCCTGGAAGTTGCGGGTAAATTG | |
| ATCAAGTATTCTGACAATGGCTCAACGCTCTCTGCGGTGAACTTCCCGGA | |
| AGTCTCGCTGCCACTGCACGGTGGGCGTCGTCTGATGCACATCGCCGAA | |
| GCCCGTCCGGGCGTGCTAACTGCGCTGAACAAAATCTTCGCCGAGCAGG | |
| GCGTCGCCATCGCCGCGCAATATCTGCAAACTTCCGCCCAGATGGGTTAT | |
| GTGGTTATTGATATTGAAGCCGACGAAGACGTTGCCGAAAAAGCGCTGC | |
| AGGCAATGAAAGCTATTCCGGGTACCATTCGCGCCCGTCTGCTGTACTA | |
| A | |
| atgaattatcagaacgacgatttacgcatcaaagaaatcaaagagttacttcctcctgtcgcattgctggaaaaattccccg | 48 |
| ctactgaaaatgccgcgaatacggttgcccatgcccgaaaagcgatccataagatcctgaaaggtaatgatgatcgcctg | |
| ttggttgtgattggcccatgctcaattcatgatcctgtcgcggcaaaagagtatgccactcgcttgctggcgctgcgtgaag | |
| agctgaaagatgagctggaaatcgtaatgcgcgtctattttgaaaagccgcgtaccacggtgggctggaaagggctgat | |
| taacgatccgcatatggataatagcttccagatcaacgacggtctgcgtatagcccgtaaattgctgcttgatattaacgac | |
| agcggtctgccagcggcaggtgagtttctcaatatgatcaccccacaatatctcgctgacctgatgagctggggcgcaat | |
| tggcgcacgtaccaccgaatcgcaggtgcaccgcgaactggcatcagggctttcttgtccggtcggcttcaaaaatggc | |
| accgacggtacgattaaagtggctatcgatgccattaatgccgccggtgcgccgcactgcttcctgtccgtaacgaaatg | |
| ggggcattcggcgattgtgaataccagcggtaacggcgattgccatatcattctgcgcggggtaaagagcctaactac | |
| agcgcgaagcacgttgctgaagtgaaagaagggctgaacaaagcaggcctgccagcacaggtgatgatcgatttcag | |
| ccatgctaactcgtccaaacaattcaaaaagcagatggatgtttgtgctgacgtttgccagcagattgccggtggcgaaaa | |
| ggccattattggcgtgatggtggaaagccatctggtggaaggcaatcagagcctcgagagcggggagccgctggcct | |
| acggtaagagcatcaccgatgcctgcatcggctgggaagataccgatgctctgttacgtcaactggcgaatgcagtaaa | |
| agcgcgtcgcgggtaa | |

What is claimed is:

1. A consortium of engineered microorganisms, comprising:
at least one upstream engineered microorganism for producing halogenated tryptophan; and
at least one downstream engineered microorganism for converting said halogenated tryptophan into a halogenated tryptophan-derived product.

2. The consortium of claim 1, wherein the at least one upstream engineered microorganism converts a carbon source to tryptophan and subsequently converts said tryptophan to halogenated tryptophan.

3. The consortium of claim 1, wherein a first upstream engineered microorganism converts a carbon source to tryptophan; and
wherein a second upstream engineered microorganism subsequently converts said tryptophan to halogenated tryptophan.

4. The consortium of claim 1, wherein the at least one upstream engineered microorganism expresses at least one halogenase.

5. The consortium of claim 4, wherein the at least one halogenase is PyrH, XsHal, Thal, Th-Hal, SttH, RebH, PrnA, AetF, or any combination thereof.

6. The consortium of claim 4, wherein the at least one upstream engineered microorganism further expresses an enzyme for generating a cofactor for the at least one halogenase.

7. The consortium of claim 6, wherein the cofactor is flavin adenine dinucleotide (FADH₂), and wherein the enzyme for generating the cofactor is a flavin reductase.

8. The consortium of claim 1, wherein the halogenated tryptophan is chlorinated or brominated.

9. The consortium of claim 1, wherein the at least one downstream engineered microorganism expresses at least one downstream enzyme.

10. The consortium of claim 9, wherein the at least one downstream enzyme is promiscuous.

11. The consortium of claim 9, wherein a first downstream enzyme converts the halogenated tryptophan into a halogenated intermediate; and
wherein a second downstream enzyme converts the halogenated intermediate into the halogenated tryptophan-derived product.

12. The consortium of claim 1, wherein the first downstream enzyme is RgnT, RgnTD, or any combination thereof; and
wherein the second downstream enzyme is RgnDC, RgnC, or any combination thereof.

13. The consortium of claim 9, wherein the at least one downstream enzyme directly converts the halogenated tryptophan to the halogenated tryptophan-derived product.

14. The consortium of claim 13, wherein the at least one downstream enzyme is iaaM, TnaA, KynA, McbB, or any combination thereof.

15. The consortium of claim 1, wherein the at least one upstream engineered microorganism and the at least one downstream engineered microorganism are separately cultured.

16. A method of making a tryptophan-derived product, the method comprising:
a) providing the consortium of claim 1;
b) exposing the at least one upstream engineered microorganism to a feedstock, thereby producing a halogenated tryptophan; and
c) exposing the at least one downstream engineered microorganism to the halogenated tryptophan, thereby converting the halogenated tryptophan to a halogenated tryptophan-derived product.

17. The method of claim 16, wherein the at least one upstream engineered microorganism and the at least one downstream engineered microorganism are separately cultured; and
wherein step b) further comprises collecting the halogenated tryptophan produced by the at least one upstream engineered microorganism.

18. The method of claim 16, wherein the feedstock comprises a carbon source, and wherein the at least one upstream engineered microorganism converts the carbon source to tryptophan; and/or
wherein the feedstock comprises tryptophan.

19. A halogenated tryptophan-derived product generated by the method of claim 16.

20. The halogenated tryptophan-derived product of claim 19, wherein the halogenated tryptophan-derived product comprises halo-tryptamine, halo-indole-3-acetamide, halo-indole, halo-N-formyl-L-kynurenine, halo-1-acetyl-3-carboxy-β-carboline, halo-2-methyl-L-tryptophan, or any combination thereof.


SEQUENCE LISTING (48 sequences)

SEQ ID NO: 1 - DNA, 19 bp, synthetic construct
  misc_feature 1..19: note = Description of sequence: artificial oligionucleotide
  source 1..19: mol_type = other DNA
ggatcctctccttgtgtga

SEQ ID NO: 2 - DNA, 26 bp, synthetic construct
  misc_feature 1..26: note = Description of sequence: artificial oligionucleotide
  source 1..26: mol_type = other DNA
taactcgagagagaatataaaaagcc

SEQ ID NO: 3 - DNA, 50 bp, synthetic construct
  misc_feature 1..50: note = Description of sequence: artificial oligionucleotide
  source 1..50: mol_type = other DNA
ggataacaatttcacacaaggagaggatccatgcttaataatgtcgttat

SEQ ID NO: 4 - DNA, 48 bp, synthetic construct
  misc_feature 1..48: note = Description of sequence: artificial oligionucleotide
  source 1..48: mol_type = other DNA
atctggctttttatattctctctcgagttattaacgcagttgggtaaa

SEQ ID NO: 5 - DNA, 43 bp, synthetic construct
  misc_feature 1..43: note = Description of sequence: artificial oligionucleotide
  source 1..43: mol_type = other DNA
acaatttcacacaaggagaggatccatggaacgccgtaaacgt

SEQ ID NO: 6 - DNA, 54 bp, synthetic construct
  misc_feature 1..54: note = Description of sequence: artificial oligionucleotide
  source 1..54: mol_type = other DNA
atctggctttttatattctctctcgagttattactgaatgcttgccagatattc

SEQ ID NO: 7 - DNA, 18 bp, synthetic construct
  misc_feature 1..18: note = Description of sequence: artificial oligionucleotide
  source 1..18: mol_type = other DNA
ttgcgccttgagcgacac

SEQ ID NO: 8 - DNA, 22 bp, synthetic construct
  misc_feature 1..22: note = Description of sequence: artificial oligionucleotide
  source 1..22: mol_type = other DNA
agcttgtcgaccctgcattagg

SEQ ID NO: 9 - DNA, 70 bp, synthetic construct
  misc_feature 1..70: note = Description of sequence: artificial oligionucleotide
  source 1..70: mol_type = other DNA
tgttattagttcgttactggaagtccagtcaccttgtcaggagtattatcattccggggatccgtcgacc

SEQ ID NO: 10 - DNA, 70 bp, synthetic construct
  misc_feature 1..70: note = Description of sequence: artificial oligionucleotide
  source 1..70: mol_type = other DNA
aaagcgggtataaattcgcccatccgttgcagatgggcgagtaagaagtagtgtaggctggagctgcttc

SEQ ID NO: 11 - DNA, 70 bp, synthetic construct
  misc_feature 1..70: note = Description of sequence: artificial oligionucleotide
  source 1..70: mol_type = other DNA
tgtaatattcacagggatcactgtaattaaaataaatgaaggattatgtaattccggggatccgtcgacc

SEQ ID NO: 12 - DNA, 71 bp, synthetic construct
  misc_feature 1..71: note = Description of sequence: artificial oligionucleotide
  source 1..71: mol_type = other DNA
tgtagggtaagagagtggctaacatccttatagccactctgtagtattaagtgtaggctggagctgcttcg

SEQ ID NO: 13 - DNA, 20 bp, synthetic construct
  misc_feature 1..20: note = Description of sequence: artificial oligionucleotide
  source 1..20: mol_type = other DNA
gttcatctttcggttggtgg

SEQ ID NO: 14 - DNA, 23 bp, synthetic construct
  misc_feature 1..23: note = Description of sequence: artificial oligionucleotide
  source 1..23: mol_type = other DNA
gtgtatatctccgaagaccgtaa

SEQ ID NO: 15 - DNA, 86 bp, synthetic construct
  misc_feature 1..86: note = Description of sequence: artificial oligionucleotide
  source 1..86: mol_type = other DNA
gtcgacgagctgttgacaattaatcatcggctcgtataatgtgtggaattgtgagcggataacaatttcacacaaggagaggatcc

SEQ ID NO: 16 - DNA, 55 bp, synthetic construct
  misc_feature 1..55: note = Description of sequence: artificial oligionucleotide
  source 1..55: mol_type = other DNA
taactcgagagagaatataaaaagccagattattaatccggcttttttattattt

SEQ ID NO: 17 - DNA, 1599 bp, synthetic construct
  misc_feature 1..1599: note = Description of sequence: artificial oligionucleotide
  source 1..1599: mol_type = other DNA
atggaacgccgtaaacgtgaacgtctgggctctctgggtcgcccgactaagaaagaactgcgtatgattcgtagcgtggtaatcgtgggcggtggcacggctggctggatgactgctagctatctgaaagcggctttcgacgatcgtattgatgttactctggttgaatccggtaacgtgcgccgtattggtgttggcgaagcaaccttctctaccgtgcgtcatttcttcgattacctgggcctggacgaacgtgaatggctgccgcgttgtgccggtggctacaaactgggcatccgttttgaaaattggtctgagccgggtgaatacttctaccacccgttcgaacgtctgcgtgttgtagacggcttcaacatggcggaatggtggctggctgttggtgaccgccgcaccagcttctccgaagcgtgctacctgactcaccgtctgtgcgaagctaaacgtgcaccacgtatgctggacggcagcctgtttgcttcccaggtggacgagagcctgggccgctccactctggcggagcagcgcgcgcaattcccgtacgcctaccacttcgatgcagatgaggtagcccgttatctgtctgaatacgcgattgcgcgtggtgtgcgtcacgtggtcgatgatgtgcagcacgttggtcaggacgaacgcggttggattagcggtgtgcacacgaaacagcacggcgagatctctggcgacctgttcgttgactgcaccggtttccgtggtctgctgatcaaccagaccctgggtggtcgtttccagtctttttccgatgtactgccgaacaaccgtgctgtcgcgctgcgcgtgccgcgcgaaaacgacgaagacatgcgtccgtataccaccgcgaccgctatgagcgctggttggatgtggaccatcccgctgttcaaacgtgatggtaacggttacgtatattccgacgaatttatctccccggaagaagctgaacgtgaactgcgcagcactgtagcgcctggccgtgacgacctggaagcaaaccacatccaaatgcgcatcggtcgtaacgaacgtacctggatcaataactgcgtggcagtcggtctgtctgcggcgttcgtcgaaccactggaaagcaccggcatcttcttcatccagcacgctatcgaacagctggttaaacacttcccgggcgaacgttgggacccggttctgatcagcgcttataacgaacgtatggcccacatggttgacggcgttaaagaatttctggttctgcattacaaaggtgctcagcgcgaggataccccgtactggaaagcggcaaaaacgcgcgctatgccagacggcctggctcgcaaactggagctgtccgcgagccacctgctggacgagcagactatctacccgtactaccacggtttcgaaacgtattcttggattaccatgaacctgggcctgggtattgtcccagaacgccctcgccctgccctgctgcacatggatccagctccggcactggcggaattcgaacgtctgcgccgtgagggcgacgaactgatcgcagcactgccgagctgctacgaatatctggcaagcattcagtaa

SEQ ID NO: 18 - DNA, 1554 bp, synthetic construct
  misc_feature 1..1554: note = Description of sequence: artificial oligionucleotide
  source 1..1554: mol_type = other DNA
atgattaattcggtattgatcgtaggtggaggtactgctggctggatgactgccgcgtatctgtcgaaagcgtttgacaagaatatcaatattaccgtcgttgagagtaaggaggtaaagaagattggggtgggcgaagccacgttttctacagttcgccattactttgattatctgggccttaacgaatctgaatggcttcctgagtgctctgggagttataaacttggtattcgcttcgaaaactgggacggacaaggaaatcatttttaccatcctttcgagcgttgggaagtagtaaagggcttcccgatttccgagtggtggctgtccaagaaattaaaggaccaacgctttgattacgatacctttttgactccacacttgtgcgaggccaaacgctcgccgcgccgcttggacgggagtttgtttgcccaatctattgacaaatcgcttggccaaagcacgcttgcagaacaacgcgctcagtacccttacgcatatcatttcgatgcagacggtgttgcgagtttccttaaacgttatgccatgaaccgtggtgtcaaacacattgaggacgatgttacacatgtggagatcgacactaacgggaacatcggttacttagaggctaaaatctatggaaaattgcgcgccgacttatacatcgattgtacggggtttaagggtcttttaatcaacaaagccttaaacgaaccatttatctcgttttccgacgtattaaagaacaaccgcgctgtggcccttcgtgtaccgcgtgagaatgagaacgatatcgaaccctatacgacagcccgtacaatgtctaatggatggcgttggacgatcccactgtacaagcgtaatggctacggttatgtttactgcaataaatatcagtctccggaggaagcagaaatggagttgcgtaaatcgatcccttatgaaaacgcggaatgcgtagccaaccatatccgcatgcgtattgggcgttcaaaccgtagctgggtcaagaactgcgttgcaattgggctttcatctgcgttcgtggaaccactggagagtacggggatcttttttattcagcacgggatcgagcaacttgtgcgcttctttccgcgctcctcgggcaacgaattgcttatcgaggaatataacacgcgcgttaatcgcgtagtcgatggggtgaaggaatttctgttacttcacttctcgctggcacagcgtaacgatactttatactggaaagaatggaagaacgtcgagttgcctaaggaacttcttaaaaagattacactggcccaggaccacttacttgataaggaaactatttacccattttaccacggattcgaagagtactcatggaacacgatgattctgggactgtgttctggttcactgaataacaaaccggcactgtccttaatgacgtccgatgaggctgatgaaatgctgctgcaacactatatgaaagctgagaacatggtgaataacctgccgacctgctatgaatatttgaagcacattcacgacttgaaaaacaaaaattaa

SEQ ID NO: 19 - DNA, 1596 bp, synthetic construct
  misc_feature 1..1596: note = Description of sequence: artificial oligionucleotide
  source 1..1596: mol_type = other DNA
atggataaccgtattaagaccgtcgttattctgggaggtgggaccgcggggtggatgacagcagcctaccttggtaaggctcttcaaaatacagtgaagattgtggtcttagaagctccgacgatccctcgtatcggggtgggagaagcaactgtaccgaatttacaacgcgccttctttgactatttggggattccggaagaagagtggatgcgtgaatgtaacgcatcttacaaaatggcagtgaagtttatcaattggcgcacccctggcgagggttcacctgatccgcgtactttggacgacggccacacagacacttttcatcaccccttcggtcttcttccaagtgccgatcagattcccctgtcacattactgggctgccaaacgcttacaaggtgagacagatgagaatttcgatgaggcgtgctttgcggacactgccatcatgaatgccaaaaaagctccacgttttcttgacatgcgccgtgcgactaattacgcgtggcatttcgacgcctctaaggtagctgcttttctgcgtaactttgcagtcacaaaacaagcggttgagcatgtcgaagacgaaatgaccgaggttttgacggatgagcgtgggtttatcactgctttgcgtacgaagtccggccgtattttgcaaggagatcttttcgtcgactgctcggggtttcgtggactgcttatcaacaaagctatggaggagccctttattgacatgtccgaccatttgttgtgcaatagcgcagtcgccacggctgtgccacacgacgatgaaaaaaatggtgtggagccgtacacaagttccatcgctatggaagctggatggacctggaagattcccatgctgggccgtttcgggagcggtcacgtgtacagcgaccatttcgcaacgcaagacgaagcgaccctggcattctcgaaattatgggggttagacccagataacaccgaattcaaccacgtacgcttccgtgtgggacgtaatcgccgtgcatgggtacgcaattgtgtgtcagtgggtcttgcaagctgcttcgtggaaccacttgaaagtagtggcatctatttcatttatgccgcgatccatatgttagcgaagcactttccggacaaaactttcgataaggttttagtcgatcgctttaaccgtgagattgaggagatgttcgatgacacacgcgatttcttgcaggctcactactattttagtccgcgcgtggacacacccttctggcgtgccaacaaggaattgaagttagcggattctattaaggacaaagtcgagacctatcgcgcaggactgccagtgaatttgcctgtgaccgacgaaggcacctattatggaaacttcgaggccgagttccgtaatttctggaccaacggatcgtactactgtatcttcgccgggcttgggctgatgccgcgcaacccattaccagctcttgcttacaagccacaaagcatcgccgaagccgagttattatttgctgacgtcaaacgcaaaggcgacacgcttgtagaatcgttgcctagcacgtatgatctgttgcgtcagcttcatggggcttcctga

SEQ ID NO: 20 - DNA, 1551 bp, synthetic construct
  misc_feature 1..1551: note = Description of sequence: artificial oligionucleotide
  source 1..1551: mol_type = other DNA
atgcttaataatgtcgttatcgtagggggaggaaccgctggctggatgacagcctcctatttaaaggctgctttcggggatcgcattgacatcactttggtcgaatcgggtcatattggcgccgttggcgttggagaggctacattctctgacattcgccattttttcgaattcctgggattaaaggagaaagactggatgccggcgtgtaatgcaacctacaagctggccgttcgctttgagaactggcgcgaaaagggacactatttttatcatccgttcgagcaaatgcgtagtgtcaacgggttcccacttacagattggtggttaaaacaaggaccgacagaccgttttgataaagattgtttcgtaatggccagtgtcattgacgccggactttctccccgtcaccaagatggcacgctgattgatcagccattcgacgagggagctgacgagatgcagggtctgacaatgtctgaacatcagggtaaaactcaattcccttacgcgtatcaatttgaggccgcattgcttgcgaaataccttacgaaatactccgtagagcgcggggtgaagcacatcgtagacgacgtgcgcgaggtatcacttgacgaccgtggatggatcacaggtgtacgtaccggagagcatggcgatttgacaggcgatctttttattgactgcacaggattccgtggtttattgttaaaccaagcccttgaagaaccctttatcagctatcaggatacgttgccaaatgactccgctgttgctttacaagtaccgatggatatggagcgtcgcggaattttaccttgtacgaccgccacggcacaggatgctggttggatttggacaatcccattaactgggcgcgttggaaccggctatgtctatgctaaagactaccttagcccggaggaagcagaacgtacattacgcgagtttgtggggcctgcggcagcggatgtggaagctaatcatattcgcatgcgtattggtcgtagccgtaactcatgggtgaaaaattgtgtggcgatcggtttgagtagcggctttgtggaaccgcttgagtcaaccggtatcttttttattcaccatgcgattgagcaattggtcaagaactttccggcggctgattggaacagtatgcaccgcgacctgtataattcggcggtgtcgcatgtcatggacggggttcgtgagtttcttgtattgcactatgtggcggcgaagcgtaacgatacacaatattggcgtgataccaagactcgtaagatccctgattcacttgcagaacgcatcgagaaatggaaggttcaactgccggatagtgagacggtatatccttactaccacgggttacctccgtactcgtatatgtgcatcttgctgggtatgggtggaattgagctgaagccgtcgcccgcgcttgctcttgccgacggcggggctgcgcaacgcgaatttgagcaaatccgtaacaaaacccaacgtctgaccgaggttttgcccaaagcgtacgactattttacccaactgcgttaa

SEQ ID NO: 21 - DNA, 1572 bp, synthetic construct
  misc_feature 1..1572: note = Description of sequence: artificial oligionucleotide
  source 1..1572: mol_type = other DNA
atgaacacacgtaatccggacaaggtggtaattgtcggcggtggtacagcaggctggatgacggcgtcttacttgaaaaaagcatttggtgagcgtgtgtcagttacacttgttgagtccggcactatcggtacggtgggggtgggtgaggctaccttttcggatattcgccacttctttgagttcctcgatctgcgtgaagaggagtggatgccggcgtgcaatgcaacttacaagctggcggtgcgctttcaagattggcagcgcccagggcatcatttttatcatcccttcgagcagatgcgctcggtcgatgggtttcctttgacggattggtggctgcaaaacggcccaaccgatcgttttgatcgcgattgcttcgtgatggcgagcctgtgcgatgcaggacggtcgcctcgctatcttaatggcagtctgcttcagcaggaattcgatgaacgcgctgaagagcctgctggtttgaccatgagtgaacaccagggcaaaacacaattcccctatgcatatcattttgaggcggcgttgctcgcggaatttctgtcaggttatagcaaagatcgtggcgttaagcacgtggtggacgaagtgctggaagtgaagctggatgatcgcggctggatctctcacgttgtcacgaaagaacacggcgacattggtggcgacctgtttgtcgattgcacgggttttcgcggcgtcctgctcaaccaggcactgggggttccgtttgtatcataccaggatacgctcccaaatgattcggcggtcgcgctgcaggtgccgcttgacatggaggctcgcggaattccaccgtatactcgggccaccgcaaaggaagcgggatggatttggacgattccactcattggtcgtatcggcaccggctacgtctacgccaaagattactgctcgccagaagaggccgagcgtacgctgcgtgaattcgtcggtcccgaagcagcggatgttgaggctaaccacattcgcatgcgtattggccgcagcgagcaaagctggaaaaataactgtgtcgccattggcctctccagcggctttgtcgaaccgctggagagcacgggtattttttttattcatcatgcgatcgagcagctggtgaaacactttccggccggcgattggcacccgcaattgcgtgccggctacaatagtgctgtggcgaacgttatggacggagtgcgcgaattcctggttctgcattatcttggcgctgcgcgtaatgacacacgctattggaaagatacgaagacgcgcgcagtgccggacgcacttgccgaacgtatcgagcgttggaaagtgcagctgccggattcggagaacgtctttccgtactatcatggtttaccaccttatagttatatggcaatcctgctgggtacaggtgcaatcggtctgcgcccgtcgccggctttggcactggcggacccggcggctgctgaaaaggaatttaccgcaattcgcgatcgtgcgcgctttctggtcgatacccttccatcacagtacgaatactttgcagccatgggtcaacgtgtctaa

SEQ ID NO: 22 - DNA, 1593 bp, synthetic construct
  misc_feature 1..1593: note = Description of sequence: artificial oligionucleotide
  source 1..1593: mol_type = other DNA
atgagtggcaagattgataaaattttgatcgtgggcggcggtaccgcgggctggatggcagcttcgtatttgggcaaggccttgcagggaactgccgatatcaccttactgcaggcgcccgacatcccaactctgggggtaggtgaggccacgattcctaatcttcagaccgccttttttgacttcttgggcattcccgaggatgaatggatgcgtgagtgtaatgccagttacaaagtggcaatcaaatttattaactggcgcacagctggcgaggggacttccgaagctcgcgaattagatggaggccccgatcatttctaccatagtttcggcctgttaaagtatcacgagcagattcccttgagtcactactggtttgaccgtagttatcgcggaaaaacagtggagccgttcgactacgcctgctataaagagccagttatccttgacgccaaccgctcaccacgtcgtctggatggctccaaggttacgaattacgcttggcactttgatgcgcatttggttgccgattttttgcgccgtttcgcgacagagaagttgggagttcgccatgtggaagatcgtgtcgagcatgttcagcgtgatgccaatggtaacattgagtccgttcgtaccgccacaggtcgtgtcttcgatgcggacttattcgttgactgctctgggttccgtggtctgctgattaataaggcaatggaggagccatttttggatatgtcggaccatctgcttaatgattctgcggtcgccacccaagtaccgcacgatgacgatgctaatggggtggaaccctttacgagcgcaatcgctatgaagagcggatggacgtggaaaatccctatgctgggacgcttcgggactggttacgtttatagttcgcgttttgcaaccgaagacgaggcggtgcgtgagttctgcgagatgtggcatttagacccggagacgcaacctctgaaccgcatccgtttccgcgtcggccgtaaccgccgtgcctgggtcggtaactgcgttagcattggcacatcaagttgtttcgtagaaccactggagtcaacagggatttacttcgtttatgcggcactttatcaactggtaaagcatttccctgataaatcgcttaaccctgttctgacagcccgtttcaatcgcgagattgaaactatgttcgacgacacccgtgactttatccaagcacacttctacttctcgccgcgcaccgatacacccttctggcgcgctaacaaggaactgcgcttggcagatggaatgcaagaaaaaattgacatgtaccgtgcaggcatggctattaatgcccctgcctcggacgacgcgcagttgtattatggcaacttcgaagaggaattccgcaatttttggaacaattcgaactattactgtgttttagcaggattaggacttgttcctgacgcgccgtctccacgtcttgctcatatgcctcaagcaacagagagtgtggatgaagtcttcggagcagtgaaagatcgtcaacgtaatcttttagaaacccttccgagtttacatgaattcttacgtcaacagcatggacgttaa

SEQ ID NO: 23 - DNA, 1617 bp, synthetic construct
  misc_feature 1..1617: note = Description of sequence: artificial oligionucleotide
  source 1..1617: mol_type = other DNA
atgaataaaccgatcaagaatatcgtgatcgttgggggcggcacggctgggtggatggcggcgtcgtatctggtacgtgcgttacagcaacaagccaacatcaccttgatcgaatccgcagcgattccccgcatcggtgtaggggaagccacgatcccgtctctgcaaaaggtgttttttgactttcttggcattcccgagcgcgagtggatgcctcaggtgaacggcgcctttaaagccgctattaaattcgtcaactggcgcaaatcaccggacccctctcgcgacgatcacttttaccatttattcggaaatgtacctaattgtgatggtgttccacttacgcattactggttgcgtaaacgtgagcagggattccaacaaccgatggagtacgcgtgctatcctcagccaggcgcattggatggaaaactggcaccttgtttatctgacggcacccgccagatgtcacacgcgtggcatttcgacgcacatttggttgccgacttccttaaacgctgggccgtggagcgtggtgtaaatcgtgtggttgatgaggtcgtagatgtgcgccttaataaccgcgggtacatctcaaatctgctgacaaaggagggacgtacgcttgaagctgacctgtttattgattgttcgggtatgcgtggtctgcttatcaatcaagctttaaaagaaccgtttatcgacatgtccgactacttgctgtgcgatagtgcggttgcaagtgcggtgcctaatgatgatgcacgtgacggggtagagccctatacctcatcgatcgcaatgaactcaggatggacttggaaaatcccgatgcttggtcgctttggatcgggctacgtcttctcgtcccacttcacttctcgtgaccaggctacggccgactttctgaagttatgggggttgtcggacaatcagccgttaaatcaaattaagtttcgtgtggggcgcaacaagcgtgcctgggtgaacaattgcgtgtcaatcggcctttcctcctgtttccttgagcctttagagtctacgggcatttacttcatctacgcggcactttaccagcttgtaaaacactttcctgatacatcttttgatccccgtctttcagacgcctttaatgcggagatcgtgcacatgttcgatgactgccgcgattttgtgcaagcgcattattttacgacctcgcgtgacgacacaccgttctggctggcgaatcgccacgacctgcgtcttagtgacgcaattaaagagaaagtgcagcgttacaaggccggtcttccccttacaaccaccagcttcgacgactcgacatattacgagaccttcgactatgaattcaagaatttctggctgaatgggaattactattgcatcttcgctggtctggggatgttgccagaccgttcccttccattattgcagcatcgcccggaaagcatcgaaaaagctgaagcgatgtttgcctcgatccgccgcgaggctgaacgcttgcgtactagtctgcccacaaattatgactacttgcgtagtcttcgtgacggcgatgccggcctttcccgcggtcaacgtggtcccaaattggctgctcaagaaagtttgtaa

SEQ ID NO: 24 - DNA, 47 bp, synthetic construct
  misc_feature 1..47: note = Description of sequence: artificial oligionucleotide
  source 1..47: mol_type = other DNA
ccactagtcagttaacgggctgtcgacctttgaaaagttcgtttaca

SEQ ID NO: 25 - DNA, 55 bp, synthetic construct
  misc_feature 1..55: note = Description of sequence: artificial oligionucleotide
  source 1..55: mol_type = other DNA
gctagctcagtcctaggtacaattgtgagcgctcacaatttcactggctgagcac

SEQ ID NO: 26 - DNA, 75 bp, synthetic construct
  misc_feature 1..75: note = Description of sequence: artificial oligionucleotide
  source 1..75: mol_type = other DNA
agctgtcaccggatgtgctttccggtctgatgagtccgtgaggacgaaacagcctctacaaataattttgtttaa

SEQ ID NO: 27 - DNA, 24 bp, synthetic construct
  misc_feature 1..24: note = Description of sequence: artificial oligionucleotide
  source 1..24: mol_type = other DNA
actagaagtaagcgaggtacacat

SEQ ID NO: 28 - DNA, 151 bp, synthetic construct
  misc_feature 1..151: note = Description of sequence: artificial oligionucleotide
  source 1..151: mol_type = other DNA
taatccaaacctgttatatgttagctgagactagttggaagtgtggctgtcctcaagcgttttagttcgtcggtcagtttcacctgatttacgtaaaaacccgcttcggcgggtttttgcttttggaggggcagaaagatgaatgactgtc

SEQ ID NO: 29 - DNA, 525 bp, synthetic construct
  misc_feature 1..525: note = Description of sequence: artificial oligionucleotide
  source 1..525: mol_type = other DNA
atgaaggtgcttgtattagccttccatccgaatatggagcaaagtgtcgtgaatcgtgcattcgccgacactcttaaagacgcgcctggcattacgttgcgtgatttatatcaggaatacccagacgaggccatcgacgtggaaaaagaacagaaactgtgtgaggaacacgaccgcattgtttttcaatttccattatattggtatagcagtccccctctgctgaaaaagtggttggatcatgttctgctgtatggttgggcatacggcactaacggtactgccttacgcggtaaggaattcatggtggcggtcagcgcaggggctccagaggaagcgtaccaggctgggggatcgaaccattatgcgattagtgaattgttgcgtccatttcaggctacctcaaactttattggtacaacttatcttcctccttatgttttttatcaggccggtaccgccggtaaatctgaattagcagagggcgcgacccagtatcgtgagcatgtgttgaagtcgttttga

SEQ ID NO: 30 - DNA, 699 bp, synthetic construct
  misc_feature 1..699: note = Description of sequence: artificial oligionucleotide
  source 1..699: mol_type = other DNA
atgacaaccttaagctgtaaagtgacctcggtagaagctatcacggataccgtatatcgtgtccgcatcgtgccagacgcggccttttcttttcgtgctggtcagtatttgatggtagtgatggatgagcgcgacaaacgtccgttctcaatggcttcgacgccggatgaaaaagggtttatcgagctgcatattggcgcttctgaaatcaacctttacgcgaaagcagtcatggaccgcatcctcaaagatcatcaaatcgtggtcgacattccccacggagaagcgtggctgcgcgatgatgaagagcgtccgatgattttgattgcgggcggcaccgggttctcttatgcccgctcgattttgctgacagcgttggcgcgtaacccaaaccgtgatatcaccatttactggggcgggcgtgaagagcagcatctgtatgatctctgcgagcttgaggcgctttcgttgaagcatcctggtctgcaagtggtgccggtggttgaacaaccggaagcgggctggcgtgggcgtactggcaccgtgttaacggcggtattgcaggatcacggtacgctggcagagcatgatatctatattgccggacgttttgagatggcgaaaattgcccgcgatctgttttgcagtgagcgtaatgcgcgggaagatcgcctgtttggcgatgcgtttgcatttatc

SEQ ID NO: 31 - DNA, 1131 bp, synthetic construct
  misc_feature 1..1131: note = Description of sequence: artificial oligionucleotide
  source 1..1131: mol_type = other DNA
atgagtaacgctaccgaggagcttaccacagtacgtgacgcgtgtgcgcgcaccctggagaatacagcccgtacgcttcatctgggcgcatcgggtaccgagtttgtcgctgccttccgtgctatgacggaccactggggcgccgcacgcccgcatgacttacccttgtcggatgtctcgcctgacggaagtcccgttgagtatgcagttgatcttggaggacttgcacctgcactgcagttcgctatggagccacttactgcaggagtccctgcacgtgatcctttagccgctcgcgccattatgcccttattagcgggacgctatggtgccgacgcgacccgttggagcgctctggctgaccgtttacttccggatgatgcccatggaccgcatgtttccatgtatggcgcggaggtccgcgcgggtgcccctatccgtttcaaggcttggttctacctgaacgtcacagggcctgacggggcgtttaatctgctttatagcgccctggaacgtatgggtactacacacctttggcctgtagtccaagcccatgtccaccgcgcaggtgaggatgtaccattcctgctttcattagatttatcagatgatccagcggcccgcgtcaaagtatactttcgtcactttgcggcagacgtcgaagaagtcgctgccgtgctgaaggcttatcccggcttcgaaccgggtgaagtccgtgcgttttgcaaggtgatgatgggaggccgtcgtcgtttttccgaccagccggctgttacatgcgtgtctttgttagacgctcagacgttcgaccgtactgctgccacgctgtacgttcctttgtggacatacgcggaacacgatggagaagtgcgccagcgtgttcaccgcactctggcagcatggcctgaagcgctttatcgctacgactccgtcttggctggcattgcccatcgcggccttgacgcaggtactggcatccataactatatcagttggcaacccggtcgcacgcgtcctcgcatgaaggtgtatttatctccggagatgcacgatgttaccccaccgccactgggggtgtcacagcaacatcacttatcggggcagactacagctcgcggccgtactgagtaa

SEQ ID NO: 32 - DNA, 867 bp, synthetic construct
  misc_feature 1..867: note = Description of sequence: artificial oligionucleotide
  source 1..867: mol_type = other DNA
atgtgtccatgcccgcattcacaagcgcagggagagacggacggcgaagcggaatggcacaatgccgcgttagactttactcatgccatgtcgtatggtgattatctgaagctggataaagtactggatgcacagtttccactttcccccgatcacaatgagatgctttttattattcaacaccagaccagcgaattatggatgaaactgatgctgcatgagcttcgtgcggcccgcgagcacgtcaaaagtgggaaattgggtcccgcattgaagatgctggctcgtgtaagccgtatttttgaccaacttgttcatgcatgggctgttttggctaccatgacgcccacagagtataatactattcgcccctatcttggtcaatcttcggggtttcaaagctatcaatatcgcgaaatcgagttcattttagggaataagaacgctacactgctgaagccacatgcccaccgtgcagagcttcttgcggctttggagcaggcgttacatacaccttcactttatgacgaggcaattcgtttaatggctgctcagggattaccggtcttccaggaacgtttggtacgtgacgcagctgcgggaacgtgttacgaagcatctgtagaggccgcatggcgtcaagtttaccaaacgccagagcgctactgggacctgtaccagcttgctgaaaaactgatcgacctggaggactcattccgccaatggcgttttcgccacgttaccaccgttgagcgcattattggattcaaaccgggaacgggtgggaccgaaggcgttgggtaccttcgttccatgttggatacaattctgttcccggaactgtggcgtttgcgctcgaacctttga

SEQ ID NO: 33 - DNA, 1800 bp, synthetic construct
  misc_feature 1..1800: note = Description of sequence: artificial oligionucleotide
  source 1..1800: mol_type = other DNA
atgttgcgcaaaggaactgtggctcttattaaccccaatcaaatccacccgccgatcgccccctatgctttagacgtattaactaccgcgcttgaagcttccggatttgaggcacacgtccttgacctgacctttcatttggatgattggcgccagacgttacgtgattacttccgcgcagaacgtccacttctggtgggcgtcacgtgccgcaacacggatactgtgtatgctttagagcagcgcccttttgtcgacggatacaaagcagtcatcgacgaagttcgccgcttaaccgctgcccccgtcgtagcaggcggcgtgggattctccacaatgccttttgctctggtggattacttcggaattgagtacggcgtaaaaggccctggcgagaagatcatttgtgacttagctcgtgccttagctgagggacgtagtgcggaccgcattcacattccaggtcttttagtaaaccgcggcccgggcaacgtcacccgcgtagcgccacctgcattagacccgcgcgcagctccggcaccatctagcagtcctagcccatcgcctgcaccgagttccagttcagcgcctgtcccggtccccttgtcctttgcggccgtcggacatcatgaaagtcgtgcttggcaggcggagacagaattaccatacactcgccgttctggagaaccctacaaggtcgataatcttcgttactaccgcgaaggcgggctgggtagtatcctgacaaaaaacgggtgtgtatataaatgctcattctgcgtcgagcctgatgccaaaggcacgcaattcgcccgccgtgggatcaccgcagttgtggacgaaatggaggctttgacagcgcaaggtatccacgatctgcatacgactgacagtgagtttaatctgtcaatcgcacattccaaaaatctgttacgtgaaatcgttcgccgtcgcgaccatgatgcgacctccccgctgcgcgacttacgcttatgggtatactgccaaccgagccctttcgatgaagagtttgcagagctgctggctgccgcaggttgtgcgggcgttaacatcggagcagatcatactcgtccagaaatgttggacggttggaaggtgacagccaaaggtacacgctattacgacttcgcggacaccgaacgtttggtacaattgtgccaccgtaatggtatgttgactatggttgaagccttattcggtatgcccggcgaaaccttagaaactatgcgcgattgtgtcgaccgcatgatggagttagatgccacggttactggcttttctctgggattacgccttcttccatatatgggtcttgcaaaaagccttgcagagcagtgcgatggagtacgcactgtccgtggtcttcaaagtaataatgctagtggcccgatcgtgttgaaacaacttcaccaatgtgatggccctattgagtatgaacgtcaatttatgtttgacgagagcggtgactttcgcttggtatgttacttctcccccgatttaccggaagctccgggtacagcagacagccctgacgggatttggcgtgcaagtgtcgacttcttgtgggaccgcattccgaaaagtgagcagtaccgtgttatgttgcccacgttaagcgggtcctcagaaaatgacaacaattacgctgataaccctttcttgacaagtttaaatcgtaaagggtacacaggagcattttgggcgcactggcgcgatcgtgaagcaattatgtcaggagctactttaccattgggcgaacttgctgaagcggtccgttaa

SEQ ID NO: 34 - DNA, 1416 bp, synthetic construct
  misc_feature 1..1416: note = Description of sequence: artificial oligionucleotide
  source 1..1416: mol_type = other DNA
atggaaaactttaaacatctccctgaaccgttccgcattcgtgttattgagccagtaaaacgtaccactcgcgcttatcgtgaagaggcaattattaaatccggtatgaacccgttcctgctggatagcgaagatgtttttatcgatttactgaccgacagcggcaccggggcggtgacgcagagcatgcaggctgcgatgatgcgcggcgacgaagcctacagcggcagtcgtagctactatgcgttagccgagtcagtgaaaaatatctttggttatcaatacaccattccgactcaccagggccgtggcgcagagcaaatctatattccggtactgattaaaaaacgcgagcaggaaaaaggcctggatcgcagcaaaatggtggcgttctctaactatttctttgataccacgcagggccatagccagatcaacggctgtaccgtgcgtaacgtctatatcaaagaagccttcgatacgggcgtgcgttacgactttaaaggcaactttgaccttgagggattagaacgcggtattgaagaagttggtccgaataacgtgccgtatatcgttgcaaccatcaccagtaactctgcaggtggtcagccggtttcactggcaaacttaaaagcgatgtacagcatcgcgaagaaatacgatattccggtggtaatggactccgcgcgctttgctgaaaacgcctatttcatcaagcagcgtgaagcagaatacaaagactggaccatcgagcagatcacccgcgaaacctacaaatatgccgatatgctggcgatgtccgccaagaaagatgcgatggtgccgatgggcggcctgctgtgcatgaaagacgacagcttctttgatgtgtacaccgagtgcagaaccctttgcgtggtgcaggaaggcttcccgacatatggcggcctggaaggcggcgcgatggagcgtctggcggtaggtctgtatgacggcatgaatctcgactggctggcttatcgtatcgcgcaggtacagtatctggtcgatggtctggaagagattggcgttgtctgccagcaggcgggcggtcacgcggcattcgttgatgccggtaaactgttgccgcatatcccggcagaccagttcccggcacaggcgctggcctgcgagctgtataaagtcgccggtatccgtgcggtagaaattggctctttcctgttaggccgcgatccgaaaaccggtaaacaactgccatgcccggctgaactgctgcgtttaaccattccgcgcgcaacatatactcaaacacatatggacttcattattgaagcctttaaacatgtgaaagagaacgcggcgaatattaaaggattaacctttacgtacgaaccgaaagtattgcgtcacttcaccgcaaaacttaaagaagtttaa

SEQ ID NO: 35 - DNA, 1380 bp, synthetic construct
  misc_feature 1..1380: note = Description of sequence: artificial oligionucleotide
  source 1..1380: mol_type = other DNA
atgaaggcggccaatgcttctagcgcggaggcctaccgtgttctgagtcgtgccttccgttttgacaacgaggatcaaaagttatggtggcactccactgcaccaatgttcgctaagatgttagaaacagcaaactataccaccccatgtcaatatcaatacttgatcacgtataaagagtgtgttattcctagcttaggctgctaccccaccaattcagcacctcgttggctttcaatccttacacgttatggcacgcctttcgagctttctctgaattgcagtaattcaatcgtccgctatacctttgagccaatcaatcagcacactggaactgataaagatccgttcaatacgcacgctatttgggagagccttcagcatcttttaccattagaaaaatctatcgaccttgagtggttccgccattttaaacatgatttaaccttgaacagtgaagagagcgcattcttggcacacaatgatcgcttagtgggcggcactattcgcacgcaaaacaaacttgcattagatttaaaggacggacgctttgcgctgaaaacgtatatttacccggccctgaaagccgttgtaaccggcaaaacgatccacgaattagtttttggctcagttcgtcgcttggctgtgcgtgagccgcgtatccttcctcctttaaacatgcttgaggaatatatccgttcacgtggttctaagtcgacagcaagcccgcgtcttgtgagctgtgatttaacgtcaccagcgaaatcgcgtatcaaaatctacttgttggagcagatggtaagtcttgaggccatggaagatttatggacattgggtggccgccgtcgtgatgcttcaaccttggaaggattaagcctggtccgcgaattgtgggacctgattcagttgtcaccgggcttaaaatcctatcctgccccttatttacccttgggtgtaatcccagatgaacgcttacctctgatggcgaactttacgctgcatcaaaatgatcctgtacccgaaccacaagtgtatttcacaacatttggcatgaacgatatggcagtggcggatgcgcttacaacttttttcgagcgtcgcgggtggagtgagatggcccgcacttacgaaacaacgttaaaatcatactatccacatgcggatcacgacaagctgaactatcttcatgcatacattagcttctcttatcgtgaccgcactccgtatttgtctgtgtacttgcagtcatttgaaaccggtgactgggcggtggctaatttgtcagaatcgaaagtcaagtgccaagacgccgcttgccagccaaccagcctgccccctgatctttctaaaaccggagtctattattctggactgcactaa

SEQ ID NO: 36 - DNA, 1419 bp, synthetic construct
  misc_feature 1..1419: note = Description of sequence: artificial oligionucleotide
  source 1..1419: mol_type = other DNA
atgtctattggagcggagattgatagtttagtacctgcgccaccgggattaaacggaaccgctgcagggtacccagctaaaactcaaaaggagctttcgaatggtgatttcgatgcacacgacggcctgtcccttgcgcaacttactccctacgacgtccttacggcggctttgccgttacctgcacctgctagtagcaccggcttttggtggcgcgaaaccggacccgtcatgtctaagcttttagctaaggcgaactatcctttgtatacgcattataaatatcttatgctgtatcacacacacatcttgccgttactgggaccgcgcccaccccttgagaattccactcacccctcgccctctaacgcgccatggcgttcattccttacggatgactttactccgcttgaaccaagttggaatgtgaacgggaatagcgaggcacagagcacaattcgccttggtattgagcccattggattcgaggcgggcgcggcggctgacccttttaaccaagccgcggttacgcagtttatgcacagctacgaggctacggaagttggcgccacattaacgttattcgaacactttcgtaatgatatgttcgttggcccagaaacttacgcagctcttcgcgcaaagatcccggaaggtgagcacacaacacaatcattccttgcattcgacttggatgccggacgtgttaccacgaaagcgtatttcttccctattcttatgtctttgaaaacgggacaaagcaccacgaaagtggtaagtgacagtattctgcatttagcattgaagagtgaggtctggggcgtccagacgattgccgcaatgagcgtgatggaagcgtggattggatcatacggcggagctgccaagactgaaatgatctcggttgattgtgtcaatgaggcagattcccgcattaagatttacgtccgtatgccacatacatcactgcgcaaggtcaaagaagcatattgtcttgggggtcgcttaaccgatgagaatacgaaggagggattaaagctgttagacgagctgtggcgtacggtgttcggcatcgatgatgaggacgcggaattacctcagaactcccaccgtacggctgggactatctttaattttgagttacgtccggggaagtggtttccagaacctaaagtttacttgcctgtacgtcactattgtgagtctgatatgcagatcgcttcccgtttacagactttcttcgggcgtttaggctggcacaacatggaaaaggattattgtaaacacttggaggacttgtttccccaccatccccttagctcttcaactggcacacacacattccttagtttttcctataaaaagcagaagggggtatacatgacaatgtattataatcttcgtgtatattcgacttaa

SEQ ID NO: 37 - DNA, 1473 bp, synthetic construct
  misc_feature 1..1473: note = Description of sequence: artificial oligionucleotide
  source 1..1473: mol_type = other DNA
atgagtcaggtaattaagaaaaaacgcaacacattcatgatcggaaccgaatacatcttgaatagtacacaacttgaagaggctattaagtcttttgtgcacgacttctgtgcagaaaagcacgaaattcatgaccagccagttgtcgttgaagccaaagagcaccaagaggacaagatcaaacaaattaaaattcccgagaaagggcgtcctgtaaacgaagtagtatcagagatgatgaacgaagtgtaccgctatcgtggcgatgctaatcacccacgcttcttttctttcgtgcccggaccggccagtagcgtctcctggcttggcgacattatgacgagtgcttataacatccacgcgggaggttccaaattagcgcccatggtcaattgtatcgaacaggaagtattgaagtggttagcgaaacaagttgggtttacggagaaccccggtggggttttcgtatctggtggaagtatggcgaacattaccgctttaactgccgcccgcgacaataagttaaccgacattaatctgcatcttggcaccgcttacatctcggaccagacgcattccagcgtcgccaaggggcttcgtattattgggatcaccgactctcgtatccgtcgcattcccacgaactcacattttcaaatggataccaccaaacttgaggaggcaattgagacggacaagaaatctgggtatattccgttcgtagtaattggcacagcgggtactacaaacacgggaagcattgatccgctgacagagatttccgcgttgtgtaaaaaacacgacatgtggtttcacattgacggggcgtacggcgcgtccgtgttattgagcccaaagtacaaaagtttgcttaccgggacaggcttggcggattctatctcgtgggacgcacacaagtggttatttcagacgtacgggtgtgcgatggtcttggttaaggacattcgcaacttatttcactcgttccatgtaaaccctgagtacttgaaggatctggagaacgatattgataacgtaaacacctgggacattggaatggaattgacccgcccagcgcgtggcctgaagctgtggctgactctgcaggttttgggttcagacttaatcggctccgcgatcgagcacggattccagttagcggtgtgggcagaagaagctttgaatcccaaaaaggattgggaaattgtgtcgcccgcacagatggctatgatcaactttcgttacgcaccaaaagaccttacaaaagaagaacaggacatccttaatgaaaaaatttcccatcgcatcttagaatccggatacgcagcgatcttcaccacagtgcttaacggtaaaacagtgctgcgtatctgtgcgattcacccagaggccactcaagaagatatgcagcataccattgatctgttagatcaatacgggcgtgagatttacaccgaaatgaagaaagcctga

SEQ ID NO: 38 - DNA, 1674 bp, synthetic construct
  misc_feature 1..1674: note = Description of sequence: artificial oligionucleotide
  source 1..1674: mol_type = other DNA
atgtatgatcattttaacagcccctcaattgatattttatatgattacggtccctttttgaagaaatgtgagatgacaggcgggatcgggtcgtattctgcaggcacaccaacgccgcgcgtagccattgttggggctggtatcagcggattggtcgccgccactgaattattacgcgcgggggttaaagacgtggttttatacgaatcgcgcgatcgtatcggaggccgtgtttggtcacaggtgttcgatcagactcgtcctcgttatattgccgaaatgggagcaatgcgttttccaccatctgctacaggtttgttccattacttgaaaaaatttggaatctctactagcactacttttcccgaccccggggtagtagacacggaattgcattatcgcgggaagcgctaccactggccagccgggaaaaagcctcccgagctgttccgtcgcgtgtacgaaggatggcaatccttattgagcgagggttacctgctggaagggggctcattggttgctcccctggatatcaccgcaatgttaaagtctggccgtctggaagaggcggcgattgcgtggcaaggatggttaaacgtctttcgtgactgttcgttttacaacgctattgtctgtatctttactggtcgccatcctcctgggggagatcgttgggcgcgccctgaggatttcgagttattcggctcgttggggattggatccgggggtttcttgccggtcttccaagcaggttttaccgagatcttgcgcatggtaattaatggttatcaatcagaccagcgcctgattcccgatgggatctcatccttagccgcgcgtttggcagaccagtctttcgacggaaaggcacttcgtgaccgcgtatgtttctctcgtgtaggacgcatctcacgtgaagcggaaaagatcattattcaaaccgaagccggagagcaacgtgtgttcgaccgtgttattgttacttcttctaaccgtgctatgcagatgattcattgtttgactgattccgagtcattcctttcccgtgatgtagcacgtgctgtccgtgagacccatttgacaggatcttctaaattgtttattcttacccgcactaaattttggatcaagaataaattgccgacgacaattcaatctgacggccttgttcgcggggtgtactgtcttgactatcagccagacgagccggagggtcatggcgttgtccttctttcgtatacttgggaagacgatgctcaaaagatgttagccatgcctgataagaagacccgctgtcaggttcttgttgatgatctggcagccatccatcccacttttgctagttatctgctgccggttgacggcgactatgagcgttacgtattgcatcatgactggcttactgacccgcatagtgccggcgccttcaagttgaattacccaggggaagacgtatattcgcaacgcctgtttttccagccgatgacagcgaattcgcctaacaaagacacgggcttgtacttagcgggctgttcttgttcatttgcgggtgggtggatcgagggcgcggtccaaacggccctgaacagtgcttgtgccgtcctgcgctccacaggtggtcagcttagtaaaggcaacccgcttgactgtattaacgcatcttaccgctattaa

SEQ ID NO: 39 - DNA, 1626 bp, synthetic construct
  misc_feature 1..1626: note = Description of sequence: artificial oligionucleotide
  source 1..1626: mol_type = other DNA
atgaatacatttacgtccaattcctccgatttaacaaccacggcgacggaaacctcctcattttcaaccctttacttgctgagtacccttcaggctttcgtagcaatcactctggttatgcttctgaagaagttaatgacagacccaaataagaaaaaaccgtaccttccacccggcccaacagggtggccgatcattgggatgatcccaacaatgttgaagagtcgccccgtgtttcgctggttgcacagtattatgaagcaactgaatacagagattgcatgcgtaaaattaggtaacacacacgtaattacagtaacgtgcccaaagatcgcacgcgagatcttaaaacagcaagatgcactttttgcttctcgcccacttacgtacgctcagaagattttatcaaatggatacaagacatgcgttatcacaccgtttggcgaccagttcaaaaagatgcgtaaggttgtgatgacagaattggtgtgtccagcccgtcatcgctggcttcatcagaaacgtagcgaggagaacgaccacttaacagcgtgggtctacaatatggtcaaaaattcggggtcagttgattttcgttttatgacccgtcactattgcgggaacgccattaaaaagctgatgtttggaactcgtacatttagtaagaacacagcgcctgatggtggacctacagtggaggacgttgaacacatggaggccatgtttgaggcgttaggttttactttcgctttctgcatctccgactatcttcccatgctgacgggtttggacttgaacgggcatgaaaagatcatgcgtgagtcatcggccattatggacaagtatcacgacccaattatcgacgagcgtatcaagatgtggcgtgaaggaaagcgcacgcaaattgaagatttcttagatatctttatcagcatcaaagacgaacaagggaatcctttattaacagccgacgaaattaaaccgactattaaagagttggtgatggcagccccagacaacccctctaacgccgtagagtgggcgatggcggaaatggtgaacaaaccagagatcctgcgtaaagccatggaagaaattgatcgcgtcgtcggaaaagagcgcttggtccaagaatctgatatcccgaagttgaattatgttaaggcgatcctgcgtgaagccttccgtttacatccggttgctgctttcaatttaccgcatgtagccttaagcgatacaacagtcgcgggataccacatccccaaaggttcccaggtgttgttgtcacgctatggactgggtcgcaaccctaaggtgtgggccgatccgttgtgtttcaagccagagcgtcacttgaacgaatgctcagaagtcacgttaactgagaatgatttacgctttatctctttctctacaggaaaacgcggttgcgctgcccccgccctggggactgccttaaccactatgatgcttgctcgccttcttcaagggttcacctggaaactgcccgagaatgaaacccgtgtggaactgatggaaagttcccatgacatgtttctggccaagccacttgtcatggtcggagatttgcgtttgcccgaacatttatatcccactgttaaataa

SEQ ID NO: 40 - DNA, 1008 bp, synthetic construct
  misc_feature 1..1008: note = Description of sequence: artificial oligionucleotide
  source 1..1008: mol_type = other DNA
atgggatcctcccaccaccatcatcatcatagttccggtttagttcctagaggatcacatatgatgagacaaatagaaatcgagtgggtccagcctggtattactgttacagcagacctaagttgggaaagaaacccagagcttgcagagttattatggactggactactaccatataacagtttacagaaccatgcactagtgtccggtaaccacctgtaccacttgatagcagacccccgtttagtgtatactgaggcccgttacaaggaggatagaactaagtcccccgatggcactgtcttcttaagccagcttcagcatctagcagtgaagtatggacccctaacagaatacctgccagcagcaccagttggctcagtggtgccagaggatattgacgcattgagagaggcaggaagagcctgttggaaggcagcttgggaaacaaaacaacccatcgaagtcagggtccgtagaaaaggcgaagctgtaaccgattttgctctacctaggactcctccagttgatcatcctggtgtccaaaagctagttgaggaaatacaagacgagactgaaagagtgtggataactcccccagctgagatcgtagacatgcaccaaggaagaattgcaagcagagccggtagctacgatcaatatttcagcactctggtatttttgaatggagaggtcaggcctcttggatactgcgccctaaacggtcttttaaagatttgccgtacaactgatctgactctaaacgatttgaagcgtattactccaacttttataaagactcccgcagaatttttgggttacaccggtctggacacactttggaggttcacacagcaggtcctgactttattaccagatgtcgaaaccagagaacagtattttgcacttgttaacgcactggcactgtatgccaacatgttgaatacttggaacctacacttttttccctggcagcatggtaccgattacagataccttgatgcataa

SEQ ID NO: 41 - DNA, 54 bp, synthetic construct
  misc_feature 1..54: note = Description of sequence: artificial oligionucleotide
  source 1..54: mol_type = other DNA
gctagctcagtcctaggtacaattacagccatcgtacgagccctggctgagcac

SEQ ID NO: 42 - DNA, 24 bp, synthetic construct
  misc_feature 1..24: note = Description of sequence: artificial oligionucleotide
  source 1..24: mol_type = other DNA
actagctgccgcagacccgcacat

SEQ ID NO: 43 - DNA, 6531 bp, synthetic construct
  misc_feature 1..6531: note = Description of sequence: artificial oligionucleotide
  source 1..6531: mol_type = other DNA
atgcaaacacaaaaaccgactctcgaactgctaacctgcgaaggcgcttatcgcgacaatcccaccgcgctttttcaccagttgtgtggggatcgtccggcaacgctgctgctggaattcgcagatatcgacagcaaagatgatttaaaaagcctgctgctggtagacagtgcgctgcgcattacagctttaggtgacactgtcacaatccaggcactttccggcaacggcgaagccctcctggcactactggataacgccctgcctgcgggtgtggaaagtgaacaatcaccaaactgccgtgtgctgcgcttcccccctgtcagtccactgctggatgaagacgcccgcttatgctccctttcggtttttgacgctttccgtttattgcagaatctgttgaatgtaccgaaggaagaacgagaagccatgttcttcggcggcctgttctcttatgaccttgtggcgggatttgaagatttaccgcaactgtcagcggaaaataactgccctgatttctgtttttatctcgctgaaacgctgatggtgattgaccatcagaaaaaaagcacccgtattcaggccagcctgtttgctccgaatgaagaagaaaaacaacgtctcactgctcgcctgaacgaactacgtcagcaactgaccgaagccgcgccgccgctgccagtggtttccgtgccgcatatgcgttgtgaatgtaatcagagcgatgaagagttcggtggcgtagtgcgtttgttgcaaaaagcgattcgcgctggagaaattttccaggtggtgccatctcgccgtttctctctgccctgcccgtcaccgctggcggcctattacgtgctgaaaaagagtaatcccagcccgtacatgttttttatgcaggataatgatttcaccctatttggcgcgtcgccggaaagctcgctcaagtatgatgccaccagccgccagattgagatctacccgattgccggaacacgcccacgcggtcgtcgcgccgatggttcactggacagagatctcgacagccgtattgaactggaaatgcgtaccgatcataaagagctgtctgaacatctgatgctggttgatctcgcccgtaatgatctggcacgcatttgcacccccggcagccgctacgtcgccgatctcaccaaagttgaccgttattcctatgtgatgcacctcgtctctcgcgtagtcggcgaactgcgtcacgatcttgacgccctgcacgcttatcgcgcctgtatgaatatggggacgttaagcggtgcgccgaaagtacgcgctatgcagttaattgccgaggcggaaggtcgtcgccgcggcagctacggcggcgcggtaggttatttcaccgcgcatggcgatctcgacacctgcattgtgatccgctcggcgctggtggaaaacggtatcgccaccgtgcaagcgggtgctggtgtagtccttgattctgttccgcagtcggaagccgacgaaacccgtaacaaagcccgcgctgtactgcgcgctattgccaccgcgcatcatgcacaggagactttctgatggctgacattctgctgctcgataatatcgactcttttacgtacaacctggcagatcagttgcgcagcaatgggcataacgtggtgatttaccgcaaccatattccggcgcaaaccttaattgaacgcctggcgaccatgagcaatccggtgctgatgctttctcctggccccggtgtgccgagcgaagccggttgtatgccggaactcctcacccgcttgcgtggcaagctgcccattattggcatttgcctcggacatcaggcgattgtcgaagcttacgggggctatgtcggtcaggcgggcgaaattctccacggtaaagcctccagcattgaacatgacggtcaggcgatgtttgccggattaacaaacccgctgccggtggcgcgttatcactcgctggttggcagtaacattccggccggtttaaccatcaacgcccattttaatggcatggtgatggcagtacgtcacgatgcggatcgcgtttgtggattccagttccatccggaatccattctcaccacccagggcgctcgcctgctggaacaaacgctggcctgggcgcagcagaaactagagccagccaacacgctgcaaccgattctggaaaaactgtatcaggcgcagacgcttagccaacaagaaagccaccagctgttttcagcggtggtgcgtggcgagctgaagccggaacaactggcggcggcgctggtgagcatgaaaattcgcggtgagcacccgaacgagatcgccggggcagcaaccgcgctactggaaaacgcagcgccgttcccgcgcccggattatctgtttgctgatatcgtcggtactggcggtgacggcagcaacagtatcaatatttctaccgccagtgcgtttgtcgccgcggcctgtgggctgaaagtggcgaaacacggcaaccgtagcgtctccagtaaatctggttcgtccgatctgctggcggcgttcggtattaatcttgatatgaacgccgataaatcgcgccaggcgctggatgagttaggtgtatgtttcctctttgcgccgaagtatcacaccggattccgccacgcgatgccggttcgccagcaactgaaaacccgcaccctgttcaatgtgctggggccattgattaacccggcgcatccgccgctggcgttaattggtgtttatagtccggaactggtgctgccgattgccgaaaccttgcgcgtgctggggtatcaacgcgcggcggtggtgcacagcggcgggatggatgaagtttcattacacgcgccgacaatcgttgccgaactgcatgacggcgaaattaaaagctatcagctcaccgcagaagactttggcctgacaccctaccaccaggagcaactggcaggcggaacaccggaagaaaaccgtgacattttaacacgtttgttacaaggtaaaggcgacgccgcccatgaagcagccgtcgctgcgaacgtcgccatgttaatgcgcctgcatggccatgaagatctgcaagccaatgcgcaaaccgttcttgaggtactgcgcagtggttccgcttacgacagagtcaccgcactggcggcacgagggtaaatgatgcaaaccgttttagcgaaaatcgtcgcagacaaggcgatttgggtagaagcccgcaaacagcagcaaccgctggccagttttcagaatgaggttcagccgagcacgcgacatttttatgatgcgctacagggtgcgcgcacggcgtttattctggagtgcaagaaagcgtcgccgtcaaaaggcgtgatccgtgatgatttcgatccagcacgcattgccgccatttataaacattacgcttcggcaatttcggtgctgactgatgagaaatattttcaggggagctttaatttcctccccatcgtcagccaaatcgccccgcagccgattttatgtaaagacttcattatcgacccttaccagatctatctggcgcgctattaccaggccgatgcctgcttattaatgctttcagtactggatgacgaccaatatcgccagcttgccgccgtcgctcacagtctggagatgggggtgctgaccgaagtcagtaatgaagaggaacaggagcgcgccattgcattgggagcaaaggtcgttggcatcaacaaccgcgatctgcgtgatttgtcgattgatctcaaccgtacccgcgagcttgcgccgaaactggggcacaacgtgacggtaatcagcgaatccggcatcaatacttacgctcaggtgcgcgagttaagccacttcgctaacggttttctgattggttcggcgttgatggcccatgacgatttgcacgccgccgtgcgccgggtgttgctgggtgagaataaagtatgtggcctgacgcgtgggcaagatgctaaagcagcttatgacgcgggcgcgatttacggtgggttgatttttgttgcgacatcaccgcgttgcgtcaacgttgaacaggcgcaggaagtgatggctgcggcaccgttgcagtatgttggcgtgttccgcaatcacgatattgccgatgtggtggacaaagctaaggtgttatcgctggcggcagtgcaactgcatggtaatgaagaacagctgtatatcgatacgctgcgtgaagctctgccagcacatgttgccatctggaaagcattaagcgtcggtgaaaccctgcccgcccgcgagtttcagcacgttgataaatatgttttagacaacggccagggtggaagcgggcaacgttttgactggtcactattaaatggtcaatcgcttggcaacgttctgctggcggggggcttaggcgcagataactgcgtggaagcggcacaaaccggctgcgccggacttgattttaattctgctgtagagtcgcaaccgggcatcaaagacgcacgtcttttggcctcggttttccagacgctgcgcgcatattaaggaaaggaacaatgacaacattacttaacccctattttggtgagtttggcggcatgtacgtgccacaaatcctgatgcctgctctgcgccagctggaagaagcttttgtcagtgcgcaaaaagatcctgaatttcaggctcagttcaacgacctgctgaaaaactatgccgggcgtccaaccgcgctgaccaaatgccagaacattacagccgggacgaacaccacgctgtatctcaagcgtgaagatttgctgcacggcggcgcgcataaaactaaccaggtgctggggcaggcgttgctggcgaagcggatgggtaaaaccgaaatcatcgccgaaaccggtgccggtcagcatggcgtggcgtcggcccttgccagcgccctgctcggcctgaaatgccgtatttatatgggtgccaaagacgttgaacgccagtcgcctaacgtttttcgtatgcgcttaatgggtgcggaagtgatcccggtgcatagcggttccgcgacgctgaaagatgcctgtaacgaggcgctgcgcgactggtccggtagttacgaaaccgcgcactatatgctgggcaccgcagctggcccgcatccttatccgaccattgtgcgtgagtttcagcggatgattggcgaagaaaccaaagcgcagattctggaaagagaaggtcgcctgccggatgccgttatcgcctgtgttggcggcggttcgaatgccatcggcatgtttgctgatttcatcaatgaaaccaacgtcggcctgattggtgtggagccaggtggtcacggtatcgaaactggcgagcacggcgcaccgctaaaacatggtcgcgtgggtatctatttcggtatgaaagcgccgatgatgcaaaccgaagacgggcagattgaagaatcttactccatctccgccggactggatttcccgtctgtcggcccacaacacgcgtatcttaacagcactggacgcgctgattacgtgtctattaccgatgatgaagcccttgaagccttcaaaacgctgtgcctgcacgaagggatcatcccggcgctggaatcctcccacgccctggcccatgcgttgaaaatgatgcgcgaaaacccggataaagagcagctactggtggttaacctttccggtcgcggcgataaagacatcttcaccgttcacgatattttgaaagcacgaggggaaatctgatggaacgctacgaatctctgtttgcccagttgaaggagcgcaaagaaggcgcattcgttcctttcgtcacgctcggtgatccgggcattgagcagtcattgaaaattatcgatacgctaattgaagccggtgctgacgcgctggagttaggtatccccttctccgacccactggcggatggcccgacgattcaaaacgccactctgcgcgcctttgcggcaggtgtgactccggcacaatgttttgaaatgctggcactgattcgccagaaacacccgaccattcccattggcctgttgatgtatgccaatctggtgtttaacaaaggcattgatgagttttatgcccagtgcgaaaaagtcggcgtcgattcggtgctggttgccgatgtgccagttgaagagtccgcgcccttccgccaggccgcgttgcgtcataatgtcgcacctatcttcatctgcccgccaaatgccgatgacgacctgctgcgccagatagcctcttacggtcgtggttacacctatttgctgtcacgagcaggcgtgaccggcgcagaaaaccgcgccgcgttacccctcaatcatctggttgcgaagctgaaagagtacaacgctgcacctccattgcagggatttggtatttccgccccggatcaggtaaaagcagcgattgatgcaggagctgcgggcgcgatttctggttcggccattgttaaaatcatcgagcaacatattaatgagccagagaaaatgctggcggcactgaaagtttttgtacaaccgatgaaagcggcgacgcgcagttaa

SEQ ID NO: 44 - DNA, 58 bp, synthetic construct
  misc_feature 1..58: note = Description of sequence: artificial oligionucleotide
  source 1..58: mol_type = other DNA
ttacattaattgcgttgcgctcatttacggctagctcagtcctaggtactatgctagc

SEQ ID NO: 45 - DNA, 29 bp, synthetic construct
  misc_feature 1..29: note = Description of sequence: artificial oligionucleotide
  source 1..29: mol_type = other DNA
tctagagtcacacaggaaagtactagatg

SEQ ID NO: 46 - DNA, 25 bp, synthetic construct
  misc_feature 1..25: note = Description of sequence: artificial oligionucleotide
  source 1..25: mol_type = other DNA
tctagagaaagaggagaaatactag

SEQ ID NO: 47 - DNA, 1233 bp, synthetic construct
  misc_feature 1..1233: note = Description of sequence: artificial oligionucleotide
  source 1..1233: mol_type = other DNA
atggcaaaggtatcgctggagaaagacaagattaagtttctgctggtagaaggcgtgcaccaaaaggcgctggaaagccttcgtgcagctggttacaccaacatcgaatttcacaaaggcgcgctggatgatgaacaattaaaagaatccatccgcgatgcccacttcatcggcctgcgatcccgtacccatctgactgaagacgtgatcaacgccgcagaaaaactggtcgctattggctgtttctgtatcggaacaaaccaggttgatctggatgcggcggcaaagcgcgggatcccggtatttaacgcaccgttctcaaatacgcgctctgttgcggagctggtgattggcgaactgctgctgctattgcgcggcgtgccggaagccaatgctaaagcgcaccgtggcgtgtggaacaaactggcggcgggttcttttgaagcgcgcggcaaaaagctgggtatcatcggctacggtcatattggtacgcaattgggcattctggctgaatcgctgggaatgtatgtttacttttatgatattgaaaataaactgccgctgggcaacgccactcaggtacagcatctttctgacctgctgaatatgagcgatgtggtgagtctgcatgtaccagagaatccgtccaccaaaaatatgatgggcgcgaaagaaatttcactaatgaagcccggctcgctgctgattaatgcttcgcgcggtactgtggtggatattccggcgctgtgtgatgcgctggcgagcaaacatctggcgggggcggcaatcgacgtattcccgacggaaccggcgaccaatagcgatccatttacctctccgctgtgtgaattcgacaacgtccttctgacgccacacattggcggttcgactcaggaagcgcaggagaatatcggcctggaagttgcgggtaaattgatcaagtattctgacaatggctcaacgctctctgcggtgaacttcccggaagtctcgctgccactgcacggtgggcgtcgtctgatgcacatcgccgaagcccgtccgggcgtgctaactgcgctgaacaaaatcttcgccgagcagggcgtcgccatcgccgcgcaatatctgcaaacttccgcccagatgggttatgtggttattgatattgaagccgacgaagacgttgccgaaaaagcgctgcaggcaatgaaagctattccgggtaccattcgcgcccgtctgctgtactaa

SEQ ID NO: 48 - DNA, 1053 bp, synthetic construct
  misc_feature 1..1053: note = Description of sequence: artificial oligionucleotide
  source 1..1053: mol_type = other DNA
atgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcaatatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaa